(19) United States
(12) Patent Application Publication
(10) Pub. No.: US 2021/0322367 A1
(43) Pub. Date: Oct. 21, 2021

(54) ISOLATED OR SYNTHETIC CANNABINOID COMPOSITIONS WITH SELECTED TERPENE BLEND AND METHODS OF USE THEREOF

(71) Applicant: BUZZELET DEVELOPMENT AND TECHNOLOGIES LTD., Or-Akiva (IL)

(72) Inventors: NOA RAZ, GIZO (IL); AHARON EYAL, JERUSALEM (IL)

(21) Appl. No.: 17/359,844

(22) Filed: Jun. 28, 2021

Related U.S. Application Data
(63) Continuation-in-part of application No. PCT/IB2019/061421, filed on Dec. 28, 2019.
(60) Provisional application No. 62/786,350, filed on Dec. 29, 2018.

(51) Int. Cl. A61K 31/352; A61K 31/05; A61K 31/353; A61P 25/28; A61P 25/00; A61P 25/20; A61P 25/04; A61P 25/08; A61P 1/08; A61K 47/10; A61K 47/06; A61K 47/22

(52) U.S. Cl. CPC A61K 31/352; A61K 31/05; A61K 31/353; A61P 25/28; A61P 25/00; A61K 47/22; A61P 25/04; A61P 25/08; A61P 1/08; A61K 47/10; A61K 47/06; A61P 25/20

(57) ABSTRACT

A method of treatment comprising administering to a subject in need thereof a therapeutically effective amount of a product including (i) at least one isolated or synthetic cannabinoid, where at least a fraction of said isolated or synthetic cannabinoid is obtained by a process selected from the group consisting of processing hemp, chemical synthesis, biological synthesis, isolation from a plant source, crystallization and combinations thereof, (ii) optionally, at least one other cannabinoid, (iii) at least one primary terpene, and (iv) optionally at least three secondary terpenes.

### FIELD

The field of art to which this invention generally pertains is improving the therapeutic effect of cannabinoid-based medicine sourced from non-Cannabis sources. Improvement of non-Cannabis sourced products is obtained via terpene enrichment, to produced enhanced therapeutic effect.

### BACKGROUND

Terpenes play important roles in cannabinoid-comprising products, affecting the functionality and bioavailability of the cannabinoids and the aroma of the product. Terpenes are common in Cannabis plants and the specific terpene content affects the Cannabis chemovar medical profile. Cannabis plants provide a fine source of tetrahydrocannabinol (THC). Other cannabinoids are present in low to very low amounts.

Utilizing production from the related hemp plants or synthesizing cannabinoids may provide important sources for producing cannabinoids other than THC, presented at low to very low amounts in most Cannabis chemovars. Production from hemp, chemical synthesis and biological synthesis would provide lower cost production methods, increasing the importance of their role in cannabinoid-based medicine. Synthesized and isolated cannabinoids have been shown to produce lower therapeutic results than those obtained from their natural counterparts. Lower therapeutic results are related to a large extent to the absence of terpenes, reducing functionality and bioavailability of the cannabinoids.

Adding selected terpene compositions to the synthesized and isolated cannabinoids will address this unmet need, producing highly effective non-THC cannabinoids treatment.

### SUMMARY

According to an aspect of some embodiments of the present invention, provided is a therapeutic product comprising, (i) at least one isolated or synthetic cannabinoid, wherein at least a fraction of said isolated or synthetic cannabinoid is obtained by a process selected from the group consisting of processing hemp, chemical synthesis, biological synthesis, isolation from a plant source and combination thereof, (ii) optionally, at least one other cannabinoid, (ii) a primary terpene, and (iv) optionally at least three secondary terpenes; forming a terpene-enriched cannabinoid product with an enhanced therapeutic effect compared with that of a product comprising the same cannabinoids amount and no more than half the amount of terpene.

According to an additional aspect of some embodiments of the present invention, provided is a method for producing the product as disclosed herein, comprising providing at least one isolated or synthetic cannabinoid and blending it with a primary terpene.

According to an embodiment, provided is a method of treatment comprising administering to a subject in need thereof a therapeutically effective amount of a product comprising (i) at least one isolated or synthetic cannabinoid, wherein at least a fraction of said isolated or synthetic cannabinoid is obtained by a process selected from the group consisting of processing hemp, chemical synthesis, biological synthesis, isolation from plant source and combinations thereof, (ii) optionally, at least one other cannabinoid, (iii) a primary terpene, and (iv) optionally at least three secondary terpenes; said product having an enhanced therapeutic effect compared with that obtained by administrating a product comprising the same cannabinoids amount and no terpene.

### DETAILED DESCRIPTION

Unless indicated otherwise, the term “chemical synthesis” as used herein refers to the process wherein cannabinoid and/or cannabinoids are generated by chemical reactions, in which the starting material is different to the cannabinoid in its natural form.

Unless indicated otherwise, the term “biological synthesis” as used herein refers to the process wherein cannabinoid and/or cannabinoids are synthesized using a living organism. A living organism may include for example bacteria and/or fungi.

Unless indicated otherwise, the term “cannabinoid” as used herein, refers to any substance that acts upon a cannabinoid receptor. Such a substance may include, for example, a cannabinoid ligand such as a full agonist, partial agonist, inverse agonist, positive allosteric, negative allosteric or antagonists acting and/or modulating cannabinoid receptors.

Unless indicated otherwise, the term “biological remnant” as used herein refers to any residue derived from the organism used for said biological synthesis. This may include for example, an amino acid, peptide, protein, enzyme, lipid and cytoplasmatic membrane.

Unless indicated otherwise the term “aging” as used herein refers to getting older, e.g. reaching an age greater than 50, greater than 60, greater than 70 or greater than 80.

Unless indicated otherwise the term “child” as used herein, refers to a person under the age of 18.

Unless indicated otherwise the term “therapeutic effect applies to a child” as used herein, refers to therapeutic effect observed when the treated patient is a child. Unless indicated otherwise the term “treat” and/or “treating” as used herein, refers to treating, healing, relieving, decreasing, preventing and/or reducing the risk associated with, and/or improving the quality of life associated with.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term “cannabinoid” as used herein refers to a compound that affects the endocannabinoid system. Cannabinoids are agonists or antagonists to receptors in the endocannabinoid system.

As used herein, the term “THC” refers to THCa (tetrahydrocannabiniolic acid) and/or to THC (tetrahydrocannabiniol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid) and/or to CBDV (cannabidivarin) unless indicated otherwise.

As used herein, the term “cellulose” refers to cellulose, hemicellulose and their combinations. As used herein, the term “glycol” refers to any glycol, including ethylene glycol, polyethylene glycol, propylene glycol and polypropylene glycol. As used herein, the term “chlorophyll” refers to chlorophyll and degradation products thereof.

As used herein, “terpene” refers to both terpenes and terpenoids.

As used herein and unless indicated otherwise, the term “terpenes/cannabinoids (or terpenes to cannabinoids) weight/weight ratio” means the weight ratio between the combined amount of terpenes and the combined amount of cannabinoids.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, when a numerical value is preceded by the term “about”, the term “about” is intended to indicate +/−10% of that value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, provided is a therapeutic product comprising, (i) at least one isolated or synthetic cannabinoid, wherein at least a fraction of said isolated or synthetic cannabinoid is obtained by a process selected from the group consisting of processing hemp, chemical synthesis, biological synthesis, isolation from plant source and combinations thereof, (ii) optionally, at least one other cannabinoid, (iii) a primary terpene, and (iv) optionally at least three secondary terpenes.

According to some embodiments, the therapeutic product is a terpene-enriched cannabinoid product with an enhanced therapeutic effect compared with that of a product comprising the same cannabinoid amount and no more than half the amount of terpene.

According to a related embodiment, the therapeutic product forms a terpene-enriched cannabinoid product with an enhanced therapeutic effect compared with that of a product comprising the same cannabinoids amount and no terpene. According to a related embodiment, the therapeutic product forms a terpene-enriched cannabinoid product with an enhanced therapeutic effect compared with that of a product comprising the same cannabinoids amount and one half the amount of said primary terpene. According to a related embodiment, the therapeutic product forms a terpene-enriched cannabinoid product with an enhanced therapeutic effect compared with that of a product comprising the same cannabinoids amount and no Cannabis terpenes. According to a related embodiment, the therapeutic product forms a a terpene-enriched cannabinoid product with an enhanced therapeutic effect compared with that of a product comprising the same cannabinoids amount and 0.01% primary terpene.

According to an embodiment, the therapeutic product comprises at least two, at least three or at least four selected cannabinoids, wherein at least a fraction of said selected cannabinoids is derived from processing hemp, chemical synthesis, biological synthesis, isolation from plant source and combination thereof.

According to an embodiment, the primary terpene to cannabinoid weight/weight ratio is said product is greater than 0.05.

According to an embodiment, wherein said at least three secondary terpenes are present, a total terpenes to cannabinoids weight/weight ratio is greater than 0.05.

According to an embodiment, the product has an enhanced therapeutic effect in treating and/or healing and/or relieving and/or decreasing, preventing and/or reducing the risk associated with, and/or improving the quality of life associated with conditions and/or symptoms associated with at least one selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof. According to an embodiment, the conditions and/or symptoms are associated with aging.

According to an embodiment, the product has an enhanced therapeutic effect in treating conditions and/or symptoms associated with aging. According to a related embodiment, the primary terpene is selected from the group consisting of caryophyllene, linalool, pinene, eucalyptol, sabinene, terpineol and combinations thereof. According to an embodiment, the composition comprises at least 2 of the terpenes, at least 3, at least 4 or at least 5 of the terpenes. According to an embodiment, the primary terpene is selected from the group consisting of caryophyllene, linalool, pinene, terpineol and combinations thereof. According to an embodiment, the primary terpene is selected from the group consisting of caryophyllene, linalool, sabinene and combinations thereof.

According to an embodiment, the enhanced therapeutic effect applies to a child.

According to an embodiment, the product has an enhanced therapeutic effect in treating a child. According to a related embodiment, the primary terpene is selected from the group consisting of caryophyllene, limonene, pinene, terpineol and combinations thereof. According to an embodiment, the composition comprises at least 2 of the terpenes, at least 3, or all 4 of the terpenes. According to an embodiment, the primary terpene is selected from the group consisting of caryophyllene, limonene and combination thereof.

According to an embodiment, the conditions and/or symptoms are observed in a child.

According to an embodiment, the product is selected from the group consisting of tablets, gel capsules, medical patches, topicals, creams, varnishes, sublingual oils, sprays, edibles, beverages, suppositories, tampons, rectal candles, cigarettes, vaporizer liquids, nasal preparation, preparations containing micro and/or nano-emulsions, preparations containing micro and/or nano-particles and combinations thereof.

According to an embodiment, the product comprises at least two cannabinoids, at least three, at least four or at least five. According to an embodiment, the content of each cannabinoid in the product is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively), Cannabinol in acid or decarboxylated form (CBNa or CBN, respectively), Cannabicyclol in acid or decarboxylated form (CBLa or CBL, respectively). As used herein, the term THC refers to THCa (tetrahydrocannabiniolic acid) and/or to THC (tetrahydrocannabiniol) unless indicated otherwise. As used herein, the term CBD refers to CBDa (cannabidiolic acid) and/or to CBD (cannabidiol) unless indicated otherwise. As used herein, the term CBG refers to CBGa (cannabigerolic acid) and/or to CBG (cannabigerol) unless indicated otherwise. As used herein, the term CBN refers to CBNa (Cannabinolic acid) and/or to CBN (cannabinol) unless indicated otherwise. As used herein, the term CBC refers to CBCa (cannabichromenic acid) and/or to CBC (Cannabichromene) unless indicated otherwise. As used herein, the term CBL refers to CBLa (Cannabicycol acid) and/or to CBL (Cannabicyclol) unless indicated otherwise. As used herein, the term THCV refers to THCVa (tetrahydrocannabivarin acid) and/or to THCV (tetrahydrocannabivarin) unless indicated otherwise. As used herein, the term CBDV refers to CBDVa (cannabigerovarin acid) and/or to CBDV (cannabidivarin) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”.

According to an embodiment, at least one of the other cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the selected cannabinoids are selected from the group consisting of tetrahydrocannabiniol (THC), cannabidiol (CBD), cannabigerol (CBG), cannabichromene (CBC), tetrahydrocannabivarin (THCV), Cannabidivarin (CBDV), Cannabinol (CBN), Cannabicyclol (CBL).

According to an embodiment, at least a fraction of the selected cannabinoids is derived from processing hemp, chemical synthesis, biological synthesis, isolation from plant source and combination thereof.

According to an embodiment, the product comprises THC and/or THCa. According to an embodiment, the product comprises CBD and/or CBDa. According to an embodiment, the product comprises THC and/or THCa at a content of less than 1%, less than 0.8%, less than 0.6%, less than 0.4% or less than 0.2%. According to an embodiment, the product comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the product comprises CBG and/or CBGa. According to an embodiment, the product comprises CBN and/or CBNa. According to an embodiment, the product comprises CBC and/or CBCa. According to an embodiment, the product comprises CBL and/or CBLa. According to an embodiment, the product comprises THCV and/or THCVa. According to an embodiment, the product comprises CBDV and/or CBDVa.

According to an embodiment, the product comprises at least 5% by weight of a non-cannabinoid, non-terpene carrier. According to an embodiment, the product comprises at least 2% by weight carrier, at least 3%, at least 5%, at least 7%, at least 10%, at least 12%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term cellulose refers to cellulose, hemicellulose and their combinations.

According to an embodiment, said product comprises than 1% THC.

According to an embodiment, the product comprises a primary terpene and optionally at least one secondary terpenes, at least two, at least three, at least four, or at least five secondary terpenes. The term “terpene”, as used herein, refers to both terpenes and terpenoids. As used here, the term “primary terpene” refers to a terpene that forms at least 20% by weight of the total amount of terpenes in the product, at least 25%, at least 30%, at least 35%, at least 40%, at least 50%, at least 60%, at least 70% or at least 80%. As used here, the term “primary terpene” refers to a terpene having the greatest amount in the composition. As used here, the term “primary terpene” refers to a terpene that forms the highest terpene concentration in the composition. According to an embodiment, the product comprises multiple (e.g. two, three, or four) terpenes, each one of which forms at least 20% by weight of the total amount of terpenes in the product and each one of these terpenes is considered a primary terpene. According to an embodiment, the product comprises multiple (e.g. two, three, or four) terpenes, each one of which forms at least 15% by weight of the total amount of terpenes in the product, at least 18%, at least 22%, at least 25%, at least 30%, at least 35%, or at least 40%, and each one of these terpenes is considered a primary terpene.

As used here, the term “secondary terpene” refers to a terpene that forms at least 10 parts per million (ppm) of the product. According to an embodiment, the content of the primary terpene in the product is at least 1.3 times greater than that of any secondary terpene, at least 1.5, at least 2, at least 2.2, at least 2.5, at least 3, at least 3.5, at least 4, at least 6, at least 8, at least 10, at least 15, at least 20, at least 25, or at least 30 times greater. According to an embodiment, the content of any secondary terpene in the product is at least 1.3 times smaller than that of the primary terpene, at least 1.5, at least 2, at least 2.2, at least 2.5, at least 3, at least 3.5, at least 4, at least 6, at least 8, at least 10, at least 15, at least 20, at least 25, or at least 30 times smaller.

According to an embodiment, the terpenes to cannabinoids weight/weight ratio in the product is about 0.05 to about 1.0. According to an embodiment, the terpenes to cannabinoids weight/weight ratio in the product is about 0.1 to about 1.0. According to an embodiment, the ratio is greater than 0.05, greater than 0.06, greater than 0.07, greater than 0.08, greater than 0.09, greater than 0.1, greater than 0.12, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 1, less than 0.9, less than 0.8, less than 0.7, less than 0.6, less than 0.5, less than 0.4, less than 0.3, less than 0.2, less than 0.15, less than 0.1, less than 0.09, less than 0.08, less than 0.07, less than 0.06, or less than 0.05. According to an embodiment, the product is selected from the group consisting of plant material, e.g. Cannabis buds (also referred to as Cannabis inflorescence) or Cannabis trim, ground forms thereof, plant material preparation for vaporizers and Cannabis cigarette. According to an embodiment, the product comprises a dried plant material. According to an embodiment, the product comprises at least one selected cannabinoid, at least two, at least three or at least four selected cannabinoids. According to an embodiment, at least a fraction of said selected cannabinoid is derived from processing hemp, chemical synthesis, biological synthesis, isolation from plant source and combination thereof.

According to an embodiment, the non-cannabinoid, non-terpene carrier comprises cellulose. According to another embodiment, the non-cannabinoid, non-terpene carrier comprises less than 5% by weight cellulose.

According to an embodiment, the product is selected from the group consisting of Cannabis trichomes, Cannabis extracts, extracts and products thereof, such as tablets, gel capsules, medical patches, topicals, creams, varnishes, sublingual oils, sprays, edibles, beverages, suppositories, tampons, rectal candles, cigarettes, vaporizer liquids, nasal preparation, preparations containing micro and nano-emulsions, preparations containing micro and nano-particles and combinations thereof. According to an embodiment, said product comprises a liquid. According to an embodiment, the product comprises at least one selected cannabinoid, at least two, at least three or at least four selected cannabinoids. According to an embodiment, at least a fraction of said selected cannabinoid is derived from processing hemp, chemical synthesis, biological synthesis, isolation from plant source and combination thereof.

According to an embodiment, the primary terpene, the secondary terpenes or both are selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol, isomers thereof and combinations thereof. According to an embodiment, the primary terpene, the secondary terpenes or both, are selected from the group consisting of pinene, limonene, linalool, caryophyllene, myrcene, terpineol, eucalyptol, geraniol and combinations thereof. According to an embodiment, the primary terpene the secondary terpenes or both, are selected from the group consisting of pinene, limonene, linalool, caryophyllene, myrcene, terpineol, eucalyptol and combinations thereof. According to an embodiment, the primary terpene is selected from the group consisting of pinene, limonene, linalool, caryophyllene, myrcene, terpineol, eucalyptol, geraniol, humulene and combinations thereof. According to an embodiment, at least one of the terpenes is a-cyclic. According to an embodiment, at least one of the terpenes is cyclic. According to an embodiment, at least one of the terpenes is not found in Cannabis buds or is present there at less than 0.2%, less than 0.1%, less than 0.05% or less than 0.02%. Such terpene is referred to as “non-Cannabis terpene”. According to an embodiment, the terpene-enriched cannabinoid product comprises the non-Cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the terpenes comprise at least one monoterpene selected from the group consisting of limonene, myrcene, pinene, linalool, geraniol, terpinene camphene and isomers thereof. According to an embodiment, the terpenes comprise at least one sesquiterpene selected from the group consisting of nerolidol, caryophyllene, farnesene, Zingiberene, vetivazulene, guaiazulene, longifolene, copaene, patchoulol humulene and isomers thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof. According to an embodiment, the terpenes comprise at least one hydroxy-terpene selected from the group consisting of nerolidol, geraniol, linalool, phytol and isomers thereof. As used herein “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, the product comprises a composition, which is liquid at 30° C. According to an embodiment, the product comprises a composition, which is a suspension at 30° C. According to an embodiment, the product comprises a composition, which is essentially clear of haze or suspended solids at 30° C.

According to an embodiment, the product comprises Cannabis plant material. According to an embodiment, the product comprises Cannabis bud. According to an embodiment, the Cannabis bud forms at least 20% of the product, at least 30%, at least 40%, at least 50%, at least 60%, at least 70% or at least 80%.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment the product comprises selected tetrahydrocannabinol (THC) and, wherein selected THC is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises selected cannabidiol (CBD), wherein selected CBD in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises selected tetrahydrocannabinol (THC) and selected cannabidiol (CBD), wherein, selected THC is in a total concentration of at least 2.5% by weight, and selected CBD is in a total concentration of at least 2.5% by weight; selected THC in a total concentration of at least 3% by weight, and selected CBD in a total concentration of at least 3% by weight; selected THC in a total concentration of at least 4% by weight, and selected CBD in a total concentration of at least 4% by weight; selected THC in a total concentration of at least 5% by weight, and selected CBD in a total concentration of at least 5% by weight; selected THC in a total concentration of at least 8% by weight, and selected CBD in a total concentration of at least 8% by weight; selected THC in a total concentration of at least 10% by weight, and selected CBD in a total concentration of at least 10% by weight; selected THC in a total concentration of at least 20% by weight, and selected CBD in a total concentration of at least 20% by weight; selected THC in a total concentration of at least 30% by weight, and selected CBD in a total concentration of at least 30% by weight.

According to a related embodiment, said product comprises selected cannabigerol (CBG) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabinol (CBN) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabichromene (CBC) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabicyclol (CBL) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected tetrahydrocannabivarin (THCV) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabidivarin (CBDV) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the product comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight. According to an embodiment, the product comprises water and water content is more than 50%, more than 60%, more than 70%, more than 80%, more than 90%, or more than 95%.

According to an embodiment, the product comprises chlorophyll. According to an embodiment, the product comprises less than 0.5% chlorophyll, at least 1, % at least 5%, at least 10%, at least 15%, at least 20% chlorophyll. As used herein, the term chlorophyll refers to chlorophyll and degradation products thereof.

According to an embodiment, the product comprises cellulose. According to an embodiment, the product comprises less than 0.5% cellulose, such as less than 0.4%, less than 0.3%, less than 0.2%, less than 0.1% or less than 0.05% cellulose.

According to an embodiment, said isolated or synthetic cannabinoid is obtained by biological synthesis, wherein the product comprises a biological remnant, derived from said biological synthesis.

According to an embodiment, said isolated or synthetic cannabinoid is an isolated cannabinoid.

According to an embodiment, said isolated or synthetic cannabinoid is crystallized.

According to an embodiment, a concentration of at said isolated or synthetic cannabinoid in the product is lower than 50%, such as lower than 45%, lower than 40%, lower than 35%, lower than 30%, lower than 25%, lower than 20%, lower than 15% or even lower than 10% by weight.

According to an embodiment, the product comprises at least one flavonoid. According to an embodiment, the product comprises at least two, at least three, at least four, or at least five flavonoids. According to an embodiment, the product comprises at least one selected from the group consisting of bergamottin, apigenin, amentoflavone, quercetin and piperine. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine

According to an embodiment, the product results in an increased and/or enhanced therapeutic effect compared with that of a product comprising the same amount of cannabinoid and no terpene. According to an embodiment, the product results in an increased and/or enhanced therapeutic effect compared with that of a product comprising the same amount of cannabinoid, optionally secondary terpenes and no primary terpene. According to an embodiment, the product results in an increased and/or enhanced therapeutic effect compared with that of a product comprising the same amount of cannabinoid and optionally secondary terpenes and a smaller amount of the primary terpene, e.g. one half of that amount. According to an embodiment, the product results in an increased and/or enhanced therapeutic effect compared with that of a product comprising the same amount of cannabinoid and no Cannabis terpenes. According to an embodiment, the product results in an increased and/or enhanced therapeutic effect compared with that of a product comprising the same amount of cannabinoid and 0.01% primary terpene.

According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, said enhanced therapeutic effect comprises shorter onset time of the therapeutic effect. According to an embodiment, the primary terpene, the cannabinoids and optionally secondary terpenes are present in specific amounts, and the onset time of the therapeutic effect, as measured by methods known in the art, is at least 20% shorter than that of a product comprising no terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same cannabinoids amount and 0.01% primary terpene, at least 30%, at least 40%, at least 50%, at least 60%, or at least 70% shorter. Shorter onset time, or differently put an earlier effect, is important particularly in cases of sublingual, edible and topical delivery and in cases where a rapid effect is desired, as in treating pain.

According to an embodiment, said enhanced therapeutic effect comprises longer onset time of the therapeutic effect. According to an embodiment, the primary terpene, the cannabinoids and optionally secondary terpenes are present in specific amounts, and the onset time of the therapeutic effect, as measured by methods known in the art, is at least 20% longer than that of a product comprising the same amount of cannabinoid and no terpene, at least 30%, at least 40%, at least 50%, at least 60%, or at least 70% longer. According to an embodiment, the primary terpene, the cannabinoids and optionally secondary terpenes are present in specific amounts, and the onset time of the therapeutic effect, as measured by methods known in the art, is at least 20% longer than that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same cannabinoids amount and 0.01% primary terpene, at least 30%, at least 40%, at least 50%, at least 60%, or at least 70% shorter. According to an embodiment, products of delayed onset time are used in combination with shorter onset time to reach a sustained release effect.

According to an embodiment, said enhanced therapeutic effect comprises greater and/or increased magnitude of the therapeutic effect. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amount of cannabinoid and no terpene, at least 30%, at least 40%, at least 50%, at least 60%, or at least 70% greater. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same cannabinoids amount and 0.01% primary terpene, at least 30%, at least 40%, at least 50%, at least 60%, or at least 70% shorter. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, said enhanced therapeutic effect comprises longer and/or extended duration of the therapeutic effect. According to an embodiment, the primary terpene, the cannabinoids and optionally secondary terpenes are present in specific amounts, and the duration of the therapeutic effect, as measured by methods known in the art, is at least 20% longer compared with that of a product comprising the same amount of cannabinoid and no terpene, at least 30%, at least 40%, at least 50%, at least 60%, at least 70%, at least 80%, at least 90% or at least 100% longer. According to an embodiment, the primary terpene, the cannabinoids and optionally secondary terpenes are present in specific amounts, and the duration of the therapeutic effect, as measured by methods known in the art, is at least 20% longer compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same cannabinoids amount and 0.01% primary terpene, at least 30%, at least 40%, at least 50%, at least 60%, or at least 70% shorter. According to an embodiment, said enhanced therapeutic effect comprises smaller therapeutically effective amount of a product and/or reduced dosages of the product. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amount of cannabinoid and no terpene, at least 30%, at least 40%, at least 50%, at least 60%, at least 70%, at least 80%, at least 90% or at least 100% smaller. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises reduced secondary adverse symptoms. According to an embodiment, the primary terpene, the cannabinoids and optionally secondary terpenes are present in specific amounts, and the secondary adverse symptoms, as measured by methods known in the art, are reduced by at least 20% compared with that of a product comprising the same amount of cannabinoid and no terpene, by at least 30%, at least 40%, at least 50%, at least 60%, at least 70%, at least 80%, at least 90% or at least 100%. According to an embodiment, the primary terpene, the cannabinoids and optionally secondary terpenes are present in specific amounts, and secondary adverse symptoms, as measured by methods known in the art, are reduced by at least 20% compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and secondary adverse symptoms, as measured by methods known in the art, are reduced by at least 20% compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and secondary adverse symptoms, as measured by methods known in the art, are reduced by at least 20% compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, secondary adverse symptoms, as measured by methods known in the art, are reduced by at least 20% compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises smaller and/or reduced frequency of conditions and/or symptoms. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amount of cannabinoid and no terpene, at least 30%, at least 40%, at least 50% at least 60%, at least 70%, at least 80%, or at least 90% smaller. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the frequency of the conditions and/or symptoms, as measured by methods known in the art, is at least 20% smaller compared with that of a product comprising the same cannabinoids amount and 0.01% primary terpene, at least 30%, at least 40%, at least 50%, at least 60%, or at least 70% shorter. According to an embodiment, said enhanced therapeutic effect comprises smaller and/or reduced severity of conditions and/or symptoms. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amounts of cannabinoids and no terpene terpene, at least 30%, at least 40%, at least 50% at least 60%, at least 70%, at least 80%, or at least 90% smaller. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the severity of the conditions and/or symptoms, as measured by methods known in the art, is at least 20% smaller compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises smaller and/or reduced consumption of other drugs. According to an embodiment, the primary terpene, the cannabinoids and optionally secondary terpenes are present in specific amounts, and the consumption of other drugs is reduced by at least 20% compared with that of a product comprising the same amount of cannabinoid and no terpene, reduced by at least 30%, by at least 40%, by at least 50%, by at least 60%, by at least 70%, by at least 80%, or by at least 90%. According to an embodiment, the primary terpene, the cannabinoids and optionally secondary terpenes are present in specific amounts, and the consumption of other drugs is reduced by at least 20% compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the consumption of other drugs is reduced by at least 20% compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the consumption of other drugs is reduced by at least 20% compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the consumption of other drugs is reduced by at least 20% compared with that of a product comprising the same cannabinoids amount and 0.01% primary terpene, at least 30%, at least 40%, at least 50%, at least 60%, or at least 70% shorter. According to an embodiment, the product comprises tetrahydrocannabinol (THC) at a concentration of less than 5% by weight, and at least 0.5% by weight primary terpene. According to an embodiment, the product is applied to a child. According to an embodiment, the primary terpene is selected from the group consisting of limonene, linalool, eucalyptol, myrcene, caryophyllene, terpineol and combinations thereof. According to an embodiment, the composition comprises tetrahydrocannabinol (THC) in a concentration of less than 5%, less than 4%, less than 3%, less than 2%, or less than 1%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of limonene, linalool, myrcene and combination thereof.

According to an embodiment, the product comprises CBD and optionally THC at CBD to THC weight/weight ratio greater than 1, wherein CBD concentration is less than 20% by weight and wherein primary terpene concentration is at least 0.5% by weight. According to an embodiment, the product is applied to a child. According to an embodiment, the primary terpene is selected from the group consisting of linalool, terpineol, ocimene, humulene, caryophyllene, eucalyptol, pinene, limonene, bisabolol, guaiol and combinations thereof. According to an embodiment, the product comprises CBD and optionally THC at CBD to THC weight/weight ratio greater 2, greater than 3, greater than 4 or greater than 5. According to an embodiment, the composition comprises cannabidiol (CBD) and/or cannabidiolic acid (CBDa) in a concentration of less than 18%, less than 16%, less than 14%, less than 12%, [0090] or less than 10%. According to an embodiment, the composition comprises cannabidiol (CBD) and/or cannabidiolic acid (CBDa), wherein CBD and/or CBDa concentration is of 10 to 20%, 10 to 15%, 15 to 20%, 12-18%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of linalool, terpineol, caryophyllene and combination thereof. According to an embodiment, the primary terpene is selected from the group consisting of linalool, terpineol, ocimene and combination thereof. According to an embodiment, the primary terpene is selected from the group consisting of eucalyptol, pinene, limonene and combination thereof. According to an embodiment, the primary terpene comprises caryophyllene and/or humulene.

According to an embodiment, the therapeutic effect is generated while administering the product in a vaporizer. According to an embodiment, the therapeutic effect is generated while administering the product by inhaling. According to an embodiment, the therapeutic effect is generated while administering the product by smoking. According to an embodiment, the therapeutic effect is generated while administering the product sublingually. According to an embodiment, the therapeutic effect is generated while applying the product, topically. According to an embodiment, the therapeutic effect is generated while administering the product in suppositories, including tampons and/or rectal candles. According to an embodiment, the therapeutic effect is generated while administering the product in a spray. According to an embodiment, the therapeutic effect is generated while administering the product as an edible, as a candy, as baked good, as a drink. According to an embodiment, the therapeutic effect is generated while administering the product as vaporizer liquid nasal preparation. According to an embodiment, the therapeutic effect is generated while administering the product as micro and nano-emulsions. According to an embodiment, the therapeutic effect is generated while administering the product as micro and nano-emulsions.

According to an embodiment, the product further comprises a selected amount of herbal preparation, wherein said herbal preparation is produced by at least one selected from the group consisting of distilling and extracting. According to an embodiment, said herbal preparation is selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, origanum, Paeonia lactiflora, Panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment said therapeutic effect treats diabetes and said product comprises at least one selected from the group consisting of CBD, THC, CBC, CBG, CBN, CBL, THCV and CBDV. According to an embodiment said therapeutic effect treats diabetes and said product comprises CBD and optionally THC at CBD to THC weight/weight ratio greater than 1, greater than 2, greater than 3, greater than 4 or greater than 5. According to an embodiment said therapeutic effect treats diabetes and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats diabetes and said primary terpene is selected from the group consisting of limonene, pinene, linalool, humulene, myrcene, phytol, geraniol, bisabolol, borneol, guaiol, ocimene, fenchol, terpineol, terpinene, eucalyptol and combinations thereof. According to an embodiment, the product comprises at least 2 of these terpenes, at least 3, at least 4, or at least 5 of the terpenes. According to an embodiment, the primary terpene is selected from the group consisting of limonene, linalool, geraniol and combinations thereof. According to an embodiment, the primary terpene is selected from the group consisting of limonene, linalool, geraniol, pinene, humulene, phytol and combinations thereof. According to various embodiments said primary terpene comprises limonene, pinene, linalool, humulene, myrcene, phytol, geraniol, bisabolol, borneol, guaiol, ocimene, fenchol, terpineol, terpinene and/or eucalyptol.

According to an embodiment said therapeutic effect treats including Crohn's disease and ulcerative colitis and said product comprises at least one selected from the group consisting of CBD, THC, CBC, CBG, CBN, CBL, THCV and CBDV. According to an embodiment said therapeutic effect treats including Crohn's disease and ulcerative colitis and said product comprises CBD and optionally THC at CBD to THC weight/weight ratio greater than 1, greater than 2, greater than 3, greater than 4 or greater than 5. According to an embodiment said therapeutic effect treats inflammatory bowel disease, including Crohn's disease and ulcerative colitis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammatory bowel disease including Crohn's disease and ulcerative colitis and said primary terpene is selected from the group consisting of limonene, caryophyllene, borneol, myrcene, pinene, terpinene, isopulegol, phellandrene, carene, carvacol, terpineol, humulene, linalool, nerolidol, guaiol and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, myrcene and combinations thereof. According to an embodiment, the primary terpene is selected from the group consisting of limonene, caryophyllene, myrcene, pinene, carene, carvacol and combinations thereof. According to various embodiments said primary terpene comprises limonene, caryophyllene, borneol, myrcene, pinene, terpinene, isopulegol, phellandrene, carene, carvacol, terpineol and/or humulene. According to an embodiment, said therapeutic effect treats Crohn's disease and ulcerative colitis and the product further comprises a compound selected from the group consisting of linalyl acetate, geranyl acetate, eugenol, eugenyl acetate, piperine and combinations thereof. According to an embodiment, the product includes fenchol and fenchol forms less than 5% by weight of the total terpene content.

According to an embodiment said therapeutic effect stimulates the immune system and/or treats weakened immune system and said product comprises at least one selected from the group consisting of CBD, THC, CBC, CBG, CBN, CBL, THCV and CBDV. According to an embodiment said therapeutic effect stimulates the immune system and/or treats weakened immune system and said product comprises CBD and optionally THC at CBD to THC weight/weight ratio greater than 1, greater than 2, greater than 3, greater than 4 or greater than 5. According to an embodiment said therapeutic effect stimulates the immune system and/or treats weakened immune system and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect stimulates the immune system and/or treats weakened immune system and said primary terpene is selected from the group consisting linalool, pinene, borneol, caryophyllene, geraniol, amyrin, terpinene, myrcene, eucalyptol, humulene and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, amyrin, and combinations thereof. According to an embodiment, the primary terpene is selected from the group consisting of linalool, caryophyllene, amyrin, pinene, terpinene, myrcene and combinations thereof. According to various embodiments said primary terpene comprises linalool, pinene, borneol, caryophyllene, geraniol, amyrin, terpinene, myrcene, eucalyptol and/or humulene.

According to an embodiment said therapeutic effect treats Fibromyalgia and said product comprises at least one selected from the group consisting of CBD, THC, CBC, CBG, CBN, CBL, THCV and CBDV. According to an embodiment said therapeutic effect treats Fibromyalgia and said product comprises CBD and optionally THC at CBD to THC weight/weight ratio greater than 1, greater than 2, greater than 3, greater than 4 or greater than 5. According to an embodiment said therapeutic effect treats Fibromyalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Fibromyalgia and said primary terpene is selected from the group consisting of linalool, caryophyllene, limonene, humulene, eucalyptol, amyrin, cycloartenol, pinene, sabinene, myrcene, terpinene, borneol, bisabolol, guaiol, terpineol, ocimene, camphene and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, eucalyptol and combinations thereof. According to an embodiment, the primary terpene is selected from the group consisting of linalool, caryophyllene, eucalyptol, limonene, pinene, sabinene, myrcene and combinations thereof. According to various embodiments said primary terpene comprises linalool, caryophyllene, limonene, humulene, eucalyptol, amyrin, cycloartenol, pinene, sabinene, myrcene, terpinene, borneol and/or bisabolol. According to an embodiment, the product includes myrcene, humulene, bisabolol, borneol, limonene and/or linalool and myrcene, humulene, bisabolol, borneol, limonene and/or linalool forms less than 5% by weight of the total terpene content.

According to an embodiment said therapeutic effect treats nociceptive pain and said product comprises at least one selected from the group consisting of CBD, THC, CBC, CBG, CBN, CBL, THCV and CBDV. According to an embodiment said therapeutic effect treats nociceptive pain and said product comprises CBD and optionally THC at CBD to THC weight/weight ratio greater than 1, greater than 2, greater than 3, greater than 4 or greater than 5. According to an embodiment said therapeutic effect treats nociceptive pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats nociceptive pain and said primary terpene is selected from the group consisting of myrcene, linalool, caryophyllene, eucalyptol, cycloartenol, borneol, sabinene, camphene, terpineol, citronellol, geraniol, humulene, bisabolol, limonene, pinene, guaiol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, eucalyptol, terpineol, citronellol and combinations thereof. According to various embodiments said primary terpene comprises myrcene, linalool, caryophyllene, eucalyptol, cycloartenol, borneol, sabinene, camphene, terpineol, citronellol, geraniol, humulene, bisabolol and/or limonene.

According to an embodiment said therapeutic effect treats neuropathic pain and said product comprises at least one selected from the group consisting of CBD, THC, CBC, CBG, CBN, CBL, THCV and CBDV. According to an embodiment said therapeutic effect treats neuropathic pain and said product comprises CBD and optionally THC at CBD to THC weight/weight ratio greater than 1, greater than 2, greater than 3, greater than 4 or greater than 5. According to an embodiment said therapeutic effect treats neuropathic pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats neuropathic pain and said primary terpene is selected from the group consisting of caryophyllene, linalool, eucalyptol, amyrin, borneol, pinene, terpineol, limonene, nerolidol, geraniol, myrcene, humulene, bisabolol, guaiol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, eucalyptol and combinations thereof. According to an embodiment, the primary terpene is selected from the group consisting of caryophyllene, linalool, eucalyptol, borneol, limonene, myrcene and combinations thereof. According to an embodiment, the primary terpene is selected from the group consisting of pinene, terpineol, nerolidol and combinations thereof. According to various embodiments said primary terpene comprises caryophyllene, linalool, eucalyptol, amyrin, borneol, pinene, terpineol, limonene, nerolidol, geraniol and/or myrcene. According to an embodiment, the product includes humulene, bisabolol, borneol, limonene and/or linalool, and humulene, bisabolol, borneol, limonene and/or linalool forms less than 5% by weight of the total terpene content.

According to an embodiment said therapeutic effect treats pain and said product comprises at least one selected from the group consisting of CBD, THC, CBC, CBG, CBN, CBL, THCV and CBDV. According to an embodiment said therapeutic effect treats pain and said product comprises CBD and optionally THC at CBD to THC weight/weight ratio greater than 1, greater than 2, greater than 3, greater than 4 or greater than 5. According to an embodiment said therapeutic effect treats pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and said primary terpene is selected from the group consisting of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, eucalyptol, borneol, terpineol and combinations thereof. According to various embodiments said primary terpene comprises myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol and/or limonene. According to an embodiment, said therapeutic effect treats pain and the product comprises a compound selected from the group consisting of linalyl acetate, geranyl acetate, eugenol, eugenyl acetate, piperine, methylis-oeugenol, Methyleugenol, menthol and combination thereof. According to an embodiment, the product includes humulene, bisabolol, borneol, limonene and/or linalool, wherein humulene, bisabolol, borneol, limonene and/or linalool forms less than 5% by weight of the total terpene content.

According to an embodiment said therapeutic effect treats arm or leg pain and said product comprises at least one selected from the group consisting of CBD, THC, CBC, CBG, CBN, CBL, THCV and CBDV. According to an embodiment said therapeutic effect treats arm or leg pain and said product comprises CBD and optionally THC at CBD to THC weight/weight ratio greater than 1, greater than 2, greater than 3, greater than 4 or greater than 5. According to an embodiment said therapeutic effect treats arm or leg pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats arm or leg pain and said primary terpene is selected from the group consisting of linalool, terpinene, pinene, limonene, terpineol, myrcene, nerolidol and combinations thereof. According to an embodiment, the product comprises at least 2 of these terpenes, at least 3, at least 4, or at least 5 of the terpenes. According to an embodiment, the primary terpene is selected from the group consisting of linalool, pinene, myrcene and combinations thereof. According to an embodiment, the primary terpene is selected from the group consisting of linalool, pinene, myrcene, limonene, terpineol, nerolidol and combinations thereof. According to various embodiments said primary terpene comprises linalool, terpinene, pinene, limonene, terpineol, myrcene and/or nerolidol.

According to an embodiment said therapeutic effect treats neurodegenerative diseases including Alzheimer disease, Parkinson disease, Huntington's disease, dystonia and/or Amyotrophic lateral sclerosis (ALS) and said product comprises at least one selected from the group consisting of CBD, THC, CBC, CBG, CBN, CBL, THCV and CBDV. According to an embodiment said therapeutic effect treats neurodegenerative diseases including Alzheimer disease, Parkinson disease, Huntington's disease, dystonia and/or Amyotrophic lateral sclerosis (ALS) and said product comprises CBD and optionally THC at CBD to THC weight/weight ratio greater than 1, greater than 2, greater than 3, greater than 4 or greater than 5. According to an embodiment said therapeutic effect treats neurodegenerative diseases including Alzheimer disease, Parkinson disease, Huntington's disease, dystonia and/or Amyotrophic lateral sclerosis (ALS) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats neurodegenerative diseases including Alzheimer disease, Parkinson disease, Huntington's disease, dystonia and/or Amyotrophic lateral sclerosis (ALS) and said primary terpene is selected from the group consisting of terpineol, caryophyllene, myrcene, terpineol, linalool, humulene, eucalyptol, pinene, pulegone, cymene, ocimene, limonene, nerolidol, sabinene, carene, citronellol, geraniol, menthol, borneol, terpinene, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, pinene, terpineol and combinations thereof. According to an embodiment, the primary terpene is selected from the group consisting of caryophyllene, linalool, pinene, terpineol, myrcene, terpineol, limonene, nerolidol, sabinene and combinations thereof. According to various embodiments said primary terpene comprises terpineol, caryophyllene, myrcene, terpineol, linalool, humulene, eucalyptol, pinene, pulegone, cymene, ocimene, limonene, nerolidol, sabinene, carene, citronellol, geraniol, menthol, borneol, terpinene, bisabolol and/or fenchol. According to an embodiment said therapeutic effect treats Alzheimer disease and said the primary terpene is selected from the group consisting of pinene, eucalyptol, linalool, terpineol, myrcene, caryophyllene and combinations thereof. According to an embodiment said therapeutic effect treats Alzheimer disease and said primary terpene is selected from the group consisting of pinene, eucalyptol, linalool and combinations thereof. According to an embodiment said therapeutic effect treats Parkinson disease, Huntington's disease, dystonia and/or Amyotrophic lateral sclerosis (ALS) and said primary terpene is selected from the group consisting caryophyllene, myrcene, linalool, pinene, limonene, nerolidol, sabinene, terpineol and combinations thereof. According to an embodiment said therapeutic effect treats Parkinson disease, Huntington's disease, dystonia and/or Amyotrophic lateral sclerosis (ALS) and said primary terpene is selected from the group consisting myrcene, linalool, pinene, nerolidol and combinations thereof. According to an embodiment, said therapeutic effect treats neurodegenerative diseases including Alzheimer, Parkinson disease, Huntington's disease, dystonia and/or Amyotrophic lateral sclerosis (ALS) and the product comprises a compound selected from the group consisting of linalyl acetate, geranyl acetate, menthol, piperine, eugenol, citronellyl formate, jasmone, benzy acetate, benzyl alcohol and combination thereof. According to an embodiment, the product includes pinene, myrcene and/or terpineol, and pinene, myrcene and/or terpineol forms less than 5% by weight of the total terpene content.

According to an embodiment said therapeutic effect treats multiple sclerosis and said product comprises at least one selected from the group consisting of CBD, THC, CBC, CBG, CBN, CBL, THCV and CBDV. According to an embodiment said therapeutic effect treats multiple sclerosis and said product comprises CBD and optionally THC at CBD to THC weight/weight ratio greater than 1, greater than 2, greater than 3, greater than 4 or greater than 5. According to an embodiment said therapeutic effect treats multiple sclerosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats multiple sclerosis and said primary terpene is selected from the group consisting of caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene, geraniol and combinations thereof. According to an embodiment, the product comprises at least 2 of these terpenes, at least 3, at least 4, or at least 5 of the terpenes. According to an embodiment, the primary terpene is selected from the group consisting of caryophyllene, myrcene, linalool and combinations thereof. According to an embodiment, the primary terpene is selected from the group consisting of caryophyllene, myrcene, linalool, pinene, limonene, eucalyptol and combinations thereof. According to various embodiments said primary terpene comprises caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene and/or geraniol.

According to an embodiment said therapeutic effect treats Myasthenia gravis and said product comprises at least one selected from the group consisting of CBD, THC, CBC, CBG, CBN, CBL, THCV and CBDV. According to an embodiment said therapeutic effect treats Myasthenia gravis and said product comprises CBD and optionally THC at CBD to THC weight/weight ratio greater than 1, greater than 2, greater than 3, greater than 4 or greater than 5. According to an embodiment said therapeutic effect treats Myasthenia gravis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Myasthenia gravis and said primary terpene is selected from the group consisting of pinene, terpineol, eucalyptol, pulegone, cymene, ocimene, nerolidol, limonene, linalool, citronellol, geraniol, menthol, caryophyllene, borneol and combinations thereof. According to an embodiment, the product comprises at least 2 of these terpenes, at least 3, at least 4, or at least 5 of the terpenes. According to an embodiment, the primary terpene is selected from the group consisting of terpineol, eucalyptol, linalool and combinations thereof. According to an embodiment, the primary terpene is selected from the group consisting of terpineol, eucalyptol, linalool, pinene, nerolidol, limonene and combinations thereof. According to various embodiments said primary terpene comprises pinene, terpineol, eucalyptol, pulegone, cymene, ocimene, nerolidol, limonene, linalool, citronellol, geraniol, menthol, caryophyllene, and/or borneol. According to an embodiment, the product includes myrcene and/or pinene, and myrcene and/or pinene forms less than 5% by weight of the total terpene content.

According to an embodiment said therapeutic effect treats epilepsy and said product comprises at least one selected from the group consisting of CBD, THC, CBC, CBG, CBN, CBL, THCV and CBDV. According to an embodiment said therapeutic effect treats epilepsy and said product comprises CBD and optionally THC at CBD to THC weight/weight ratio greater than 1, greater than 2, greater than 3, greater than 4 or greater than 5. According to an embodiment said therapeutic effect treats epilepsy and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats epilepsy and said primary terpene is selected from the group consisting of linalool, terpineol, ocimene, myrcene, limonene, caryophyllene and combinations thereof. According to an embodiment, the product comprises at least 2 of these terpenes, at least 3, at least 4, or at least 5 of the terpenes. According to an embodiment, the primary terpene is selected from the group consisting of linalool, terpineol, ocimene and combinations thereof. According to various embodiments said primary terpene comprises linalool, terpineol, ocimene, myrcene, limonene and/or caryophyllene.

According to an embodiment said therapeutic effect treats cancer and/or cancer related symptom and said product comprises at least one selected from the group consisting of CBD, THC, CBC, CBG, CBN, CBL, THCV and CBDV. According to an embodiment said therapeutic effect treats cancer and/or cancer related symptom and said product comprises CBD and optionally THC at CBD to THC weight/weight ratio greater than 1, greater than 2, greater than 3, greater than 4 or greater than 5. According to an embodiment said therapeutic effect treats cancer and/or cancer related symptom and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats cancer and/or cancer related symptom and said primary terpene is selected from the group consisting of myrcene, limonene, caryophyllene, caryophyllene oxide, terpinene, citronellol, linalool, geraniol, enthol, humulene, amyrin, cycloartenol, pinene, camphene, eucalyptol, terpineol, borneol, bisabolol, nerolidol, fenchol, guaiol and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, linalool and combinations thereof. According to an embodiment, the primary terpene is selected from the group consisting of limonene, caryophyllene, linalool, citronellol, myrcene, eucalyptol, pinene and combinations thereof. According to various embodiments said primary terpene comprises myrcene, limonene, caryophyllene, caryophyllene oxide, terpinene, citronellol, linalool, geraniol, menthol, humulene, amyrin, cycloartenol, pinene, camphene, eucalyptol, terpineol and/or borneol. According to an embodiment, said therapeutic effect treats cancer and/or cancer related symptom and the product comprises a compound selected from the group consisting of linalyl acetate, geranyl acetate, eugenol, eugenyl acetate, piperine, methylis-oeugenol, Methyleugenol, menthol and combination thereof.

According to an embodiment said therapeutic effect treats stroke and/or traumatic brain injury and said product comprises at least one selected from the group consisting of CBD, THC, CBC, CBG, CBN, CBL, THCV and CBDV. According to an embodiment said therapeutic effect treats stroke and/or traumatic brain injury and said product comprises CBD and optionally THC at CBD to THC weight/weight ratio greater than 1, greater than 2, greater than 3, greater than 4 or greater than 5. According to an embodiment said therapeutic effect treats stroke and/or traumatic brain injury and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats stroke and/or traumatic brain injury and said primary terpene is selected from the group consisting of myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol, carotol and combinations thereof. According to an embodiment, the product comprises at least 2 of these terpenes, at least 3, at least 4, or at least 5 of the terpenes. According to an embodiment, the primary terpene is selected from the group consisting of myrcene, caryophyllene, limonene and combinations thereof. According to an embodiment, the primary terpene is selected from the group consisting of myrcene, caryophyllene, limonene, pinene, humulene, terpineol, thymol, eucalyptol and combinations thereof. According to various embodiments said primary terpene comprises myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol and/or carotol. According to an embodiment, the product includes fenchol and/or borneol, and fenchol and/or borneol forms less than 5% by weight of the total terpene content.

According to an embodiment said therapeutic effect treats addiction, including addiction to drugs, smoking, drinks, food, gambling, sex, and sports and said product comprises at least one selected from the group consisting of CBD, THC, CBC, CBG, CBN, CBL, THCV and CBDV. According to an embodiment said therapeutic effect treats addiction, including addiction to drugs, smoking, drinks, food, gambling, sex, and sports and said product comprises CBD and optionally THC at CBD to THC weight/weight ratio greater than 1, greater than 2, greater than 3, greater than 4 or greater than 5. According to an embodiment said therapeutic effect treats addiction, including addiction to drugs, smoking, drinks, food, gambling, sex, and sports and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats addiction, including addiction to drugs, smoking, drinks, food, gambling, sex, and sports and said primary terpene is selected from the group consisting of caryophyllene, pinene, myrcene, limonene, linalool, terpineol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, terpineol and combinations thereof. According to various embodiments said primary terpene comprises caryophyllene, pinene, myrcene, limonene, linalool and/or terpineol.

According to an embodiment said therapeutic effect treats inflammatory airway diseases including asthma and/or chronic obstructive pulmonary disease (COPD) and said product comprises at least one selected from the group consisting of CBD, THC, CBC, CBG, CBN, CBL, THCV and CBDV. According to an embodiment said therapeutic effect treats inflammatory airway diseases including asthma and/or chronic obstructive pulmonary disease (COPD) and said product comprises CBD and optionally THC at CBD to THC weight/weight ratio greater than 1, greater than 2, greater than 3, greater than 4 or greater than 5. According to an embodiment said therapeutic effect treats inflammatory airway diseases including asthma and/or chronic obstructive pulmonary disease (COPD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammatory airway diseases including asthma and/or chronic obstructive pulmonary disease (COPD) and said primary terpene is selected from the group consisting of pinene, eucalyptol, limonene, humulene, camphene, caryophyllene and combinations thereof. According to an embodiment, the product comprises at least 2 of these terpenes, at least 3, at least 4, or at least 5 of the terpenes. According to an embodiment, the primary terpene is selected from the group consisting of pinene, eucalyptol, humulene, and combinations thereof. According to various embodiments said primary terpene comprises pinene, eucalyptol, limonene, humulene, camphene and/or caryophyllene.

According to an embodiment said therapeutic effect treats cerebral palsy (CP) and said product comprises at least one selected from the group consisting of CBD, THC, CBC, CBG, CBN, CBL, THCV and CBDV. According to an embodiment said therapeutic effect treats cerebral palsy (CP) and said product comprises CBD and optionally THC at CBD to THC weight/weight ratio greater than 1, greater than 2, greater than 3, greater than 4 or greater than 5. According to an embodiment said therapeutic effect treats cerebral palsy (CP) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cerebral palsy (CP) and said primary terpene is selected from the group consisting of myrcene, pinene, linalool, humulene, nerolidol, limonene, terpineol, sabinene and combinations thereof. According to an embodiment, the product comprises at least 2 of these terpenes, at least 3, at least 4, or at least 5 of the terpenes. According to an embodiment, the primary terpene is selected from the group consisting of myrcene, pinene, nerolidol and combinations thereof. According to an embodiment, the primary terpene is selected from the group consisting of linalool, limonene, sabinene and combinations thereof. According to various embodiments said primary terpene comprises myrcene, pinene, linalool, humulene, nerolidol, limonene, terpineol and/or sabinene.

According to an embodiment said therapeutic effect treats autism and/or autism spectrum disorder and said product comprises at least one selected from the group consisting of CBD, THC, CBC, CBG, CBN, CBL, THCV and CBDV. According to an embodiment said therapeutic effect treats autism and/or autism spectrum disorder and said product comprises CBD and optionally THC at CBD to THC weight/weight ratio greater than 1, greater than 2, greater than 3, greater than 4 or greater than 5. According to an embodiment said therapeutic effect treats autism and/or autism spectrum disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats autism and/or autism spectrum disorder and said primary terpene is selected from the group consisting of limonene, caryophyllene, pinene, myrcene, humulene, citronellol, eucalyptol, linalool, terpineol, ocimene, geraniol, menthol, borneol, bisabolol, fenchol, guaiol and combinations thereof. According to an embodiment, the product comprises at least 2 of these terpenes, at least 3, at least 4, or at least 5 of the terpenes. According to an embodiment, the primary terpene is selected from the group consisting of pinene, eucalyptol, linalool and combinations thereof. According to an embodiment, the primary terpene is selected from the group consisting of pinene, eucalyptol, linalool, caryophyllene, myrcene, terpineol and combinations thereof. According to various embodiments said primary terpene comprises limonene, caryophyllene, pinene, myrcene, humulene, citronellol, eucalyptol, linalool, terpinenol, ocimene, geraniol, menthol, borneol, bisabolol and/or fenchol.

According to an embodiment said therapeutic effect treats hyperactivity disorder (ADHD), attention deficit disorder (ADD) and/or obsessive-compulsive disorder (OCD) and said product comprises at least one selected from the group consisting of CBD, THC, CBC, CBG, CBN, CBL, THCV and CBDV. According to an embodiment said therapeutic effect treats hyperactivity disorder (ADHD), attention deficit disorder (ADD) and/or obsessive-compulsive disorder (OCD) and said product comprises CBD and optionally THC at CBD to THC weight/weight ratio greater than 1, greater than 2, greater than 3, greater than 4 or greater than 5. According to an embodiment said therapeutic effect treats hyperactivity disorder (ADHD), attention deficit disorder (ADD) and/or obsessive-compulsive disorder (OCD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hyperactivity disorder (ADHD), attention deficit disorder (ADD) and/or obsessive-compulsive disorder (OCD) and said primary terpene is selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof. According to an embodiment, the product comprises at least 2 of these terpenes, at least 3, at least 4, or at least 5 of the terpenes. According to an embodiment said therapeutic effect treats hyperactivity disorder (ADHD) and/or attention deficit disorder (ADD) and said primary terpene is selected from the group consisting of linalool, eucalyptol, myrcene, and combinations thereof. According to an embodiment, the primary terpene is selected from the group consisting of linalool, eucalyptol, myrcene, caryophyllene, pinene, borneol and combinations thereof. According to an embodiment said therapeutic effect treats obsessive-compulsive disorder (OCD) and said primary terpene is selected from the group consisting of limonene, eucalyptol, pinene and combination thereof. According to an embodiment, the primary terpene is selected from the group consisting of linalool, caryophyllene, borneol and combination thereof. According to various embodiments said primary terpene comprises linalool, eucalyptol, terpineol, myrcene, pinene, pulegone, caryophyllene, geraniol, citronellol, menthol, caryophyllene, pinene, borneol, bisabolol and/or fenchol.

According to an embodiment said therapeutic effect treats Tourette syndrome, motor spasms, motor tics and/or vocal tics and said product comprises at least one selected from the group consisting of CBD, THC, CBC, CBG, CBN, CBL, THCV and CBDV. According to an embodiment said therapeutic effect treats Tourette syndrome, motor spasms, motor tics and/or vocal tics and said product comprises CBD and optionally THC at CBD to THC weight/weight ratio greater than 1, greater than 2, greater than 3, greater than 4 or greater than 5. According to an embodiment said therapeutic effect treats Tourette syndrome, motor spasms, motor tics and/or vocal tics and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Tourette syndrome, motor spasms, motor tics and/or vocal tics and said primary terpene is selected from the group consisting of myrcene, linalool, limonene, pinene, nerolidol, caryophyllene, eucalyptol, geraniol, menthol, borneol, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of myrcene, limonene, nerolidol and combinations thereof. According to an embodiment, the primary terpene is selected from the group consisting of linalool, pinene, eucalyptol and combinations thereof. According to various embodiments said primary terpene comprises myrcene, linalool, limonene, pinene, nerolidol, caryophyllene, eucalyptol, geraniol, menthol, borneol, bisabolol and/or fenchol.

According to an embodiment said therapeutic effect treats psoriasis and said product comprises at least one selected from the group consisting of CBD, THC, CBC, CBG, CBN, CBL, THCV and CBDV. According to an embodiment said therapeutic effect treats psoriasis and said product comprises CBD and optionally THC at CBD to THC weight/weight ratio greater than 1, greater than 2, greater than 3, greater than 4 or greater than 5. According to an embodiment said therapeutic effect treats psoriasis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats psoriasis and said primary terpene is selected from the group consisting of linalool, caryophyllene, limonene and combinations thereof. According to an embodiment, the product comprises at least 2 of these terpenes or all 3 of the terpenes. According to an embodiment, the primary terpene is selected from the group consisting of linalool, caryophyllene, limonene and combinations thereof. According to various embodiments said primary terpene comprises linalool, caryophyllene and/or limonene.

According to an embodiment said therapeutic effect treats dermatophytes and said product comprises at least one selected from the group consisting of CBD, THC, CBC, CBG, CBN, CBL, THCV and CBDV. According to an embodiment said therapeutic effect treats dermatophytes and said product comprises CBD and optionally THC at CBD to THC weight/weight ratio greater than 1, greater than 2, greater than 3, greater than 4 or greater than 5. According to an embodiment said therapeutic effect treats dermatophytes and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats dermatophytes and said primary terpene is selected from the group consisting of limonene, caryophyllene, caryophyllene oxide, nerolidol, eucalyptol, terpinene, cycloartenol, amyrin, campene and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, caryophyllene oxide and combinations thereof. According to an embodiment, the primary terpene is selected from the group consisting of limonene, caryophyllene, caryophyllene oxide, nerolidol, eucalyptol, terpinene and combinations thereof. According to various embodiments said primary terpene comprises limonene, caryophyllene, caryophyllene oxide, nerolidol, eucalyptol, terpinene, cycloartenol, amyrin and/or campene.

According to an embodiment said therapeutic effect treats Candida infection and said product comprises at least one selected from the group consisting of CBD, THC, CBC, CBG, CBN, CBL, THCV and CBDV. According to an embodiment said therapeutic effect treats Candida infection and said product comprises CBD and optionally THC at CBD to THC weight/weight ratio greater than 1, greater than 2, greater than 3, greater than 4 or greater than 5. According to an embodiment said therapeutic effect treats Candida infection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Candida infection and said primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ionone and combinations thereof. According to an embodiment, the product comprises at least 2 of these terpenes, at least 3, at least 4, or at least 5 of the terpenes. According to an embodiment, the primary terpene is selected from the group consisting of thymol, carvacrol, eugenol and combinations thereof. According to an embodiment, the primary terpene is selected from the group consisting of thymol, carvacrol, eugenol, nerolidol, linalool, farnesol, citronellol, geraniol, citral and combinations thereof. According to various embodiments said primary terpene comprises pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and/or ionone.

According to an embodiment said therapeutic effect treats leishmaniasis and said product comprises at least one of CBD, THC, CBC, CBG, CBN, CBL, THCV and CBDV. According to an embodiment said therapeutic effect treats leishmaniasis and said product comprises CBD and optionally THC at CBD to THC weight/weight ratio greater than 1, greater than 2, greater than 3, greater than 4 or greater than 5. According to an embodiment said therapeutic effect treats leishmaniasis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats leishmaniasis and said primary terpene is selected from the group consisting of linalool, nerolidol, pinene, caryophyllene and/or eucalyptol. According to an embodiment said primary terpene is linalool and/or nerolidol.

According to an embodiment said therapeutic effect treats Methycillin-resistant Staphylococcus aureus (MRSA) and said product comprises at least one of CBD, THC, CBC, CBG, CBN, CBL, THCV and CBDV. According to an embodiment said therapeutic effect treats Methycillin-resistant Staphylococcus aureus (MRSA) and said product comprises CBD and optionally THC at CBD to THC weight/weight ratio greater than 1, greater than 2, greater than 3, greater than 4 or greater than 5. According to an embodiment said therapeutic effect treats Methycillin-resistant Staphylococcus aureus (MRSA) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Methycillin-resistant Staphylococcus aureus (MRSA) and said primary terpene is selected from the group consisting of pinene, caryophyllene, caryophyllene oxide, limonene, nerolidol, cycloartenol, amyrin and combinations thereof. According to an embodiment, the product comprises at least 2 of these terpenes, at least 3, at least 4, or at least 5 of the terpenes. According to an embodiment, the primary terpene is selected from the group consisting of pinene, caryophyllene, limonene, amyrin and combinations thereof. According to various embodiments said primary terpene comprises pinene, caryophyllene, caryophyllene oxide, limonene, nerolidol, cycloartenol and/or amyrin.

According to an embodiment said therapeutic effect treats malaria and said product comprises at least one of CBD, THC, CBC, CBG, CBN, CBL, THCV and CBDV. According to an embodiment said therapeutic effect treats malaria and said product comprises CBD and optionally THC at CBD to THC weight/weight ratio greater than 1, greater than 2, greater than 3, greater than 4 or greater than 5. According to an embodiment said therapeutic effect treats malaria and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats malaria and said primary terpene is selected from the group consisting of caryophyllene, nerolidol, pinene, eucalyptol, limonene and combinations thereof. According to an embodiment, the product comprises at least 2 of these terpenes, at least 3, at least 4, or at least 5 of the terpenes. According to an embodiment, the primary terpene is selected from the group consisting of caryophyllene, nerolidol, pinene and combinations thereof. According to various embodiments said primary terpene comprises caryophyllene, nerolidol, pinene, eucalyptol and/or limonene.

According to an embodiment said therapeutic effect treats acne and said product comprises at least one of CBD, THC, CBC, CBG, CBN, CBL, THCV and CBDV. According to an embodiment said therapeutic effect treats acne and said product comprises CBD and optionally THC at CBD to THC weight/weight ratio greater than 1, greater than 2, greater than 3, greater than 4 or greater than 5. According to an embodiment said therapeutic effect treats acne) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acne and said primary terpene is selected from the group consisting of limonene, linalool, nerolidol, pinene, terpineol and combinations thereof. According to an embodiment, the product comprises at least 2 of these terpenes, at least 3, at least 4, or all 5 of the terpenes. According to an embodiment, the primary terpene is selected from the group consisting of limonene, pinene, terpineol and combinations thereof. According to various embodiments said primary terpene comprises limonene, linalool, nerolidol, pinene and/or terpineol.

According to an embodiment said therapeutic effect treats allergy and said product comprises at least one of CBD, THC, CBC, CBG, CBN, CBL, THCV and CBDV. According to an embodiment said therapeutic effect treats allergy and said product comprises CBD and optionally THC at CBD to THC weight/weight ratio greater than 1, greater than 2, greater than 3, greater than 4 or greater than 5. According to an embodiment said therapeutic effect treats allergy and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats allergy and said primary terpene is selected from the group consisting of camphene, pinene, borneol, caryophyllene, geraniol and combinations thereof. According to an embodiment, the product comprises at least 2 of these terpenes, at least 3, at least 4, or all 5 of the terpenes. According to an embodiment, the primary terpene is selected from the group consisting of camphene, pinene, caryophyllene and combinations thereof. According to various embodiments said primary terpene comprises camphene, pinene, borneol, caryophyllene and/or geraniol.

According to an embodiment said therapeutic effect treats osteoporosis and said product comprises at least one of CBD, THC, CBC, CBG, CBN, CBL, THCV and CBDV. According to an embodiment said therapeutic effect treats osteoporosis and said product comprises CBD and optionally THC at CBD to THC weight/weight ratio greater than 1, greater than 2, greater than 3, greater than 4 or greater than 5. According to an embodiment said therapeutic effect treats osteoporosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats osteoporosis and said the primary terpene is selected from the group consisting of caryophyllene, caryophyllene oxide, carene, eucalyptol, sabinene, pinene, limonene, borneol, thymol, camphor and combinations thereof. According to an embodiment, the product comprises at least 2 of these terpenes, at least 3, at least 4, or at least 5 of the terpenes. According to an embodiment, the primary terpene is selected from the group consisting of caryophyllene, carene, camphor and combinations thereof. According to an embodiment, the primary terpene is selected from the group consisting of caryophyllene, carene, camphor, pinene, limonene, borneol and combinations thereof. According to various embodiments said primary terpene comprises caryophyllene oxide, eucalyptol, sabinene, pinene, limonene, thymol and/or camphor. According to an embodiment, said therapeutic effect treats osteoporosis and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats sleep disorder and/or insomnia and said product comprises at least one of CBD, THC, CBC, CBG, CBN, CBL, THCV and CBDV. According to an embodiment said therapeutic effect treats sleep disorder and/or insomnia and said product comprises CBD and optionally THC at CBD to THC weight/weight ratio greater than 1, greater than 2, greater than 3, greater than 4 or greater than 5. According to an embodiment said therapeutic effect treats sleep disorder and/or insomnia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats sleep disorder and/or insomnia and said the primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol, citral and combinations thereof. According to an embodiment, the product comprises at least 2 of these terpenes, at least 3, at least 4, or at least 5 of the terpenes. According to an embodiment, the primary terpene is selected from the group consisting of linalool, terpineol, myrcene and combinations thereof. According to an embodiment, the primary terpene is selected from the group consisting of linalool, terpineol, myrcene, caryophyllene, terpinene, borneol and combinations thereof. According to an embodiment, the primary terpene comprises terpineol, citronellol and/or linalool. According to various embodiments said primary terpene comprises terpineol, terpinene, citronellol, eucalyptol, nerolidol, sabinene, pinene, limonene, borneol, fenchol, bisabolol, phytol and/or humulene. According to an embodiment, said therapeutic effect treats sleep disorder and/or insomnia and the product further comprises a compound selected from the group consisting of linalyl acetate and/or chamazulene. According to an embodiment, the product includes caryophyllene, and caryophyllene forms less than 5% by weight of the total terpene content.

According to an embodiment said therapeutic effect treats anxiety, irritability, nervousness, restless, stress, depression, general discomfort, mood problems and/or affect disorder and said product comprises at least one of CBD, THC, CBC, CBG, CBN, CBL, THCV and CBDV. According to an embodiment said therapeutic effect treats anxiety, irritability, nervousness, restless, stress, depression, general discomfort, mood problems and/or affect disorder and said product comprises CBD and optionally THC at CBD to THC weight/weight ratio greater than 1, greater than 2, greater than 3, greater than 4 or greater than 5. According to an embodiment said therapeutic effect treats anxiety, irritability, nervousness, restless, stress, depression, general discomfort, mood problems and/or affect disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats anxiety, irritability, nervousness, restless, stress, depression, general discomfort, mood problems and/or affect disorder and said the primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol, phellandrene and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, terpineol, caryophyllene, pinene, myrcene and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol phellandrene and/or fenchol. According to an embodiment, said therapeutic effect treats anxiety, irritability, nervousness, restless, stress, depression, general discomfort, mood problems and/or affect disorder and the product further comprises a compound selected from the group consisting of linalyl acetate, geranyl acetate, menthol, piperine, eugenol, citronellyl formate, jasmone, benzy acetate, benzyl alcohol and combinations thereof. According to an embodiment, the product includes bisabolol, nerolidol, terpinene, myrcene and/or pinene bisabolol, nerolidol, terpinene, myrcene and/or pinene, and bisabolol, nerolidol, terpinene and/or pinene forms less than 5% by weight of the total terpene content.

According to an embodiment said therapeutic effect treats difficulty in concentration, memory loss, and/or dementia and said product comprises at least one of CBD, THC, CBC, CBG, CBN, CBL, THCV and CBDV. According to an embodiment said therapeutic effect treats difficulty in concentration, memory loss, and/or dementia and said product comprises CBD and optionally THC at CBD to THC weight/weight ratio greater than 1, greater than 2, greater than 3, greater than 4 or greater than 5. According to an embodiment said therapeutic effect treats difficulty in concentration, memory loss, and/or dementia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats difficulty in concentration, memory loss, and/or dementia and said the primary terpene is selected from the group consisting of pinene, pulegone, eucalyptol, terpineol, cymene, ocimene, limonene, linalool, caryophyllene, humulene, carene and combinations thereof. According to an embodiment, the product comprises at least 2 of these terpenes, at least 3, at least 4, or at least 5 of the terpenes. According to an embodiment, the primary terpene is selected from the group consisting of eucalyptol, pinene, limonene and combinations thereof. According to an embodiment, the primary terpene is selected from the group consisting of eucalyptol, pinene, limonene, terpineol, humulene, carene and combinations thereof. According to various embodiments said primary terpene comprises pinene, pulegone, eucalyptol, terpineol, cymene, ocimene, limonene, linalool, caryophyllene, humulene and/or carene.

According to an embodiment said therapeutic effect treats muscle pain, myalgia, spasticity, muscle tension, cramps and/or spasms and said product comprises at least one of CBD, THC, CBC, CBG, CBN, CBL, THCV and CBDV. According to an embodiment said therapeutic effect treats muscle pain, myalgia, spasticity, muscle tension, cramps and/or spasms and said product comprises CBD and optionally THC at CBD to THC weight/weight ratio greater than 1, greater than 2, greater than 3, greater than 4 or greater than 5. According to an embodiment said therapeutic effect treats muscle pain, myalgia, spasticity, muscle tension, cramps and/or spasms and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats muscle pain, myalgia, spasticity, muscle tension, cramps and/or spasms and said primary terpene is selected from the group consisting of myrcene, pinene, nerolidol, linalool, humulene, caryophyllene, limonene, bisabolol, sabinene, citral, eucalyptol, terpineol, geraniol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, limonene and combinations thereof. According to an embodiment, the primary terpene is selected from the group consisting of myrcene, pinene, limonene, nerolidol, linalool, caryophyllene, sabinene and combinations thereof. According to an embodiment, the primary terpene is selected from the group consisting of myrcene, pinene, nerolidol, linalool, humulene and combinations thereof. According to various embodiments said primary terpene comprises myrcene, pinene, nerolidol, linalool, humulene, caryophyllene, limonene, bisabolol, sabinene, citral, eucalyptol, geraniol and/or terpineol. According to an embodiment said therapeutic effect treats cramps and/or spasms and said primary terpene is selected from the group consisting of myrcene, pinene, nerolidol, sabinene, eucalyptol and combinations thereof. According to an embodiment said primary terpene is selected from the group consisting of myrcene, linalool, eucalyptol and combinations thereof.

According to an embodiment said therapeutic effect treats fatigue, weakness, decreased mental energy, decreased physical energy, dizziness and/or deficits in balance and said product comprises at least one of CBD, THC, CBC, CBG, CBN, CBL, THCV and CBDV. According to an embodiment said therapeutic effect treats fatigue, weakness, decreased mental energy, decreased physical energy, dizziness and/or deficits in balance and said product comprises CBD and optionally THC at CBD to THC weight/weight ratio greater than 1, greater than 2, greater than 3, greater than 4 or greater than 5. According to an embodiment said therapeutic effect treats fatigue, weakness, decreased mental energy, decreased physical energy, dizziness and/or deficits in balance and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fatigue, weakness, decreased mental energy, decreased physical energy, dizziness and/or deficits in balance and said the primary terpene is selected from the group consisting of pinene, limonene, terpinene, eucalyptol, myrcene, terpineol, linalool, caryophyllene, borneol and combinations thereof. According to an embodiment, the product comprises at least 2 of these terpenes, at least 3, at least 4, or at least 5 of the terpenes. According to an embodiment, the primary terpene is selected from the group consisting of pinene, terpinene, eucalyptol and combinations thereof. According to an embodiment, the primary terpene is selected from the group consisting of limonene, myrcene, borneol and combinations thereof. According to various embodiments said primary terpene comprises terpinene, myrcene, borneol and/or caryophyllene.

According to an embodiment said therapeutic effect treats itchy skin, pruritus and/or chronic pruritus, wrinkles, stretch marks and/or skin burns and said product comprises at least one of CBD, THC, CBC, CBG, CBN, CBL, THCV and CBDV. According to an embodiment said therapeutic effect treats itchy skin, pruritus and/or chronic pruritus, wrinkles, stretch marks and/or skin burns and said product comprises CBD and optionally THC at CBD to THC weight/weight ratio greater than 1, greater than 2, greater than 3, greater than 4 or greater than 5. According to an embodiment said therapeutic effect treats itchy skin, pruritus and/or chronic pruritus, wrinkles, stretch marks and/or skin burns and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats itchy skin, pruritus and/or chronic pruritus, wrinkles, stretch marks and/or skin burns and said the primary terpene is selected from the group consisting of linalool limonene, nerolidol, pinene terpineol, myrcene, geraniol, nerol, citronellol, farnesol, carotol, sabinene and combinations thereof. According to an embodiment, the product comprises at least 2 of these terpenes, at least 3, at least 4, or at least 5 of the terpenes. According to an embodiment, the primary terpene is selected from the group consisting of linalool, terpineol, geraniol and combinations thereof. According to an embodiment said therapeutic effect treats skin burns and said the primary terpene is linalool and/or sabinene. According to an embodiment said therapeutic effect treats wrinkles, stretch marks and said the primary terpene is selected from the group consisting of geraniol, linalool, citronellol and combinations thereof. According to various embodiments said primary terpene comprises limonene, nerolidol, pinene myrcene, nerol, citronellol, farnesol, linalool, terpineol, geraniol and/or carotol. According to an embodiment, said therapeutic effect treats itchy skin, pruritus and/or chronic pruritus, wrinkles, stretch marks and/or skin burns and the product comprises a compound selected from the group consisting of linalyl acetate, citronellyl formate, eugenol, benzy acetate, benzyl alcohol, and combinations thereof.

According to an embodiment said therapeutic effect treats headache and/or migraine and said product comprises at least one of CBD, THC, CBC, CBG, CBN, CBL, THCV and CBDV. According to an embodiment said therapeutic effect treats headache and/or migraine and said product comprises CBD and optionally THC at CBD to THC weight/weight ratio greater than 1, greater than 2, greater than 3, greater than 4 or greater than 5. According to an embodiment said therapeutic effect treats headache and/or migraine and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats headache and/or migraine and said the primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, limonene, nerolidol, fenchol and combinations thereof. According to various embodiments said primary terpene comprises of myrcene, linalool, caryophyllene, eucalyptol, limonene, nerolidol, fenchol, humulene, guaiol, borneol, pinene, terpinene, terpineol and/or bisabolol. According to various embodiments, said primary terpene comprises myrcene, linalool, caryophyllene, eucalyptol, limonene, nerolidol, fenchol, humulene, guaiol, borneol, bisabolol, pinene, ocimene, camphene, terpineol and combinations thereof. According to an embodiment, the product includes linalool, bisabolol and/or humulene wherein linalool, bisabolol and/or humulene forms less than 5% by weight of the total terpene content.

According to an embodiment said therapeutic effect treats weight gain and/or reduces appetite and said product comprises at least one of CBD, THC, CBC, CBG, CBN, CBL, THCV and CBDV. According to an embodiment said therapeutic effect treats weight gain and/or reduces appetite and said product comprises CBD and optionally THC at CBD to THC weight/weight ratio greater than 1, greater than 2, greater than 3, greater than 4 or greater than 5. According to an embodiment said therapeutic effect treats weight gain and/or reduces appetite and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats weight gain and/or reduces appetite and said the primary terpene is selected from the group consisting of eucalyptol, caryophyllene, limonene, humulene, phytol and combinations thereof. According to an embodiment, the product comprises at least 2 of these terpenes, at least 3, at least 4, or at least 5 of the terpenes. According to an embodiment, the primary terpene is selected from the group consisting of eucalyptol, limonene, humulene and combinations thereof. According to various embodiments said primary terpene comprises caryophyllene, eucalyptol, limonene, humulene and/or phytol.

According to an embodiment said therapeutic effect treats digestive problems, intestinal disorders, gastrointestinal pain, abdominal pain (colic), pelvic pain, constipation and/or diarrhea and said product comprises at least one of CBD, THC, CBC, CBG, CBN, CBL, THCV and CBDV. According to an embodiment said therapeutic effect treats digestive problem, intestinal disorder, gastrointestinal pain, abdominal pain (colic), pelvic pain, constipation and/or diarrhea and said product comprises CBD and optionally THC at CBD to THC weight/weight ratio greater than 1, greater than 2, greater than 3, greater than 4 or greater than 5. According to an embodiment said therapeutic effect treats digestive problem, intestinal disorder, gastrointestinal pain, abdominal pain (colic), pelvic pain, constipation and/or diarrhea and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats digestive problem, intestinal disorder, gastrointestinal pain, abdominal pain (colic), pelvic pain, constipation and/or diarrhea and said the primary terpene is selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaiol, terpineol and combinations thereof. According to an embodiment, the product comprises at least 2 of these terpenes, at least 3, at least 4, or at least 5 of the terpenes. According to an embodiment, the primary terpene is selected from the group consisting of limonene, sabinene, caryophyllene and combinations thereof. According to an embodiment, the primary terpene is selected from the group consisting of limonene, sabinene, caryophyllene, citronellol, linalool, myrcene, nerolidol and combinations thereof. According to various embodiments said primary terpene comprises limonene, cymene, ocimene, terpinene, caryophyllene, citronellol, sabinene, linalool, pinene, myrcene, germacrene, geraniol, nerolidol, menthol, humulene, fenchol and/or borneol. According to an embodiment, said therapeutic effect treats digestive problem, intestinal disorder, gastrointestinal pain, abdominal pain (colic), pelvic pain, constipation and/or diarrhea and the product further comprises piperine. According to an embodiment, the product includes pinene, bisabolol and/or limonene, and pinene, bisabolol and/or limonene forms less than 5% by weight of the total terpene content.

According to an embodiment said therapeutic effect treats lack of appetite, nausea, vomiting and/or anorexia and said product comprises at least one of CBD, THC, CBC, CBG, CBN, CBL, THCV and CBDV. According to an embodiment said therapeutic effect treats lack of appetite, nausea, vomiting and/or anorexia and said product comprises CBD and optionally THC at CBD to THC weight/weight ratio greater than 1, greater than 2, greater than 3, greater than 4 or greater than 5. According to an embodiment said therapeutic effect treats lack of appetite, nausea, vomiting and/or anorexia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats lack of appetite, nausea, vomiting and/or anorexia and said the primary terpene is selected from the group consisting of linalool, citronellol, geraniol, menthol, humulene, bisabolol, borneol, fenchol, linalool, limonene, terpineol, pinene, caryophyllene, myrcene, cymene, terpinene, valencene and combinations thereof. According to an embodiment, the product comprises at least 2 of these terpenes, at least 3, at least 4, or at least 5 of the terpenes. According to an embodiment, the primary terpene is selected from the group consisting of citronellol, linalool caryophyllene and combinations thereof. According to an embodiment, the primary terpene is selected from the group consisting of citronellol, linalool caryophyllene, limonene, myrcene, nerolidol and combinations thereof According to various embodiments said primary terpene comprises linalool, citronellol, geraniol, menthol, humulene, bisabolol, borneol, fenchol, linalool, limonene, terpineol, pinene, caryophyllene, myrcene, cymene and/or terpinene. According to an embodiment, the product includes pinene. borneol, fenchol, terpinene and/or terpineol, and pinene, bisabolol and/or limonene forms less than 5% by weight of the total terpene content.

According to an embodiment said therapeutic effect treats joint pain, joint stiffness, arthralgia, arthritis and/or osteoarthritis and said product comprises at least one of CBD, THC, CBC, CBG, CBN, CBL, THCV and CBDV. According to an embodiment said therapeutic effect treats joint pain, joint stiffness, arthralgia, arthritis and/or osteoarthritis and said product comprises CBD and optionally THC at CBD to THC weight/weight ratio greater than 1, greater than 2, greater than 3, greater than 4 or greater than 5. According to an embodiment said therapeutic effect treats joint pain, joint stiffness, arthralgia, arthritis and/or osteoarthritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats a joint pain, joint stiffness, arthralgia, arthritis and/or osteoarthritis and said the primary terpene is selected from the group consisting of myrcene, linalool, caryophyllene, eucalyptol, pinene, nerolidol, sabinene, limonene, terpinene, menth-2-en-1-ol, terpineol, geraniol, guaiol, humulene, bisabolol, borneol, fenchol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, sabinene and combinations thereof. According to an embodiment, the primary terpene is selected from the group consisting of myrcene, pinene, sabinene linalool, caryophyllene, limonene and combinations thereof. According to various embodiments said primary terpene comprises myrcene, linalool, caryophyllene, eucalyptol, pinene, nerolidol, sabinene, limonene, terpinene, menth-2-en-1-ol, geraniol, guaiol, humulene, bisabolol, borneol, fenchol 1 and/or terpineol. According to an embodiment, said therapeutic effect treats joint pain, joint stiffness, arthralgia, arthritis and/or osteoarthritis and the product comprises a compound selected from the group consisting of linalyl acetate, geranyl acetate, eugenol, methylis-oeugenol, menthol, eugenyl acetate, piperine and combinations thereof. According to an embodiment, the product optionally includes humulene, pinene, bisabolol, borneol, fenchol, guaiol, linalool, ocimene, terpineol, terpinene and/or caryophyllene, and humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, night sweat, palpitation, irritability and/or restlessness and said product comprises at least one of CBD, THC, CBC, CBG, CBN, CBL, THCV and CBDV. According to an embodiment said therapeutic effect treats hot flashes, night sweat, palpitation, irritability and/or restlessness and said product comprises CBD and optionally THC at CBD to THC weight/weight ratio greater than 1, greater than 2, greater than 3, greater than 4 or greater than 5. According to an embodiment said therapeutic effect treats hot flashes, night sweat, palpitation, irritability and/or restlessness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hot flashes, night sweat, palpitation, irritability and/or restlessness and said the primary terpene is selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof. According to an embodiment, the product comprises at least 2 of these terpenes, at least 3, at least 4, or at least 5 of the terpenes. According to an embodiment, the primary terpene is selected from the group consisting of linalool, limonene, eucalyptol and combinations thereof. According to an embodiment, the primary terpene is selected from the group consisting of linalool, limonene, terpineol, geraniol, citronellol, eucalyptol and combinations thereof. According to various embodiments said primary terpene comprises caryophyllene, myrcene, humulene, pinene, linalool, limonene, nerolidol, terpinene, terpineol, geraniol, carene, citronellol, eucalyptol, sabinene, myrtenol, bisabolol, borneol and/or fenchol. According to an embodiment, said therapeutic effect treats hot flashes, night sweat, palpitation, irritability and/or restlessness and the product further comprises a compound selected from the group consisting of linalyl acetate, geranyl acetate, citronellyl formate and combinations thereof. According to an embodiment, the product optionally includes pinene, bisabolol and/or terpineol, and pinene, bisabolol and/or terpineol forms less than 5% by weight of the total terpene content.

According to an embodiment said therapeutic effect treats inflammation, oxidative stress and/or paresthesia and said product comprises at least one of CBD, THC, CBC, CBG, CBN, CBL, THCV and CBDV. According to an embodiment said therapeutic effect treats inflammation, oxidative stress and/or paresthesia and said product comprises CBD and optionally THC at CBD to THC weight/weight ratio greater than 1, greater than 2, greater than 3, greater than 4 or greater than 5. According to an embodiment said therapeutic effect treats inflammation, oxidative stress and/or paresthesia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammation, oxidative stress and/or paresthesia and said the primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof. According to an embodiment, the product comprises at least 2 of these terpenes, at least 3, at least 4, or at least 5 of the terpenes. According to an embodiment, the primary terpene is selected from the group consisting of myrcene, limonene, thymol and combinations thereof. According to an embodiment, the primary terpene is selected from the group consisting of caryophyllene, pinene, linalool, humulene, myrcene, limonene, thymol and combinations thereof According to various embodiments said primary terpene comprises caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, n and/or nerolidol. According to an embodiment, said therapeutic effect treats inflammation, oxidative stress and/or paresthesia and the product further comprises a compound selected from the group consisting of linalyl acetate, geranyl acetate, eugenol, eugenyl acetate, piperine and combinations thereof. According to an embodiment, the product optionally includes fenchol and/or borneol, and fenchol and/or borneol forms less than 5% by weight of the total terpene content.

According to an embodiment said therapeutic effect treats cardiovascular disease, palpitation and tachycardia and said product comprises at least one of CBD, THC, CBC, CBG, CBN, CBL, THCV and CBDV. According to an embodiment said therapeutic effect treats cardiovascular disease, palpitation and tachycardia and said product comprises CBD and optionally THC at CBD to THC weight/weight ratio greater than 1, greater than 2, greater than 3, greater than 4 or greater than 5. According to an embodiment said therapeutic effect treats cardiovascular disease, palpitation and tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cardiovascular disease, palpitation and tachycardia and said the primary terpene is selected from the group consisting of linalool, camphene, limonene, pinene, borneol and combinations thereof. According to an embodiment, the product comprises at least 2 of these terpenes, at least 3, at least 4, or all 5 of the terpenes. According to an embodiment, the primary terpene is selected from the group consisting of camphene, limonene, pinene and combinations thereof. According to various embodiments said primary terpene comprises linalool, camphene, limonene, pinene and/or borneol.

According to an embodiment, the enhanced therapeutic effect comprises reducing side effects associated with selected cannabinoid consumption or from the other cannabinoid consumption. According to an embodiment, the primary terpene comprises geraniol, and the side effect comprises hypoglycemia and/or reduction of glucose level. According to an embodiment, the primary terpene is selected from the group consisting of pinene, terpinene, linalool, eucalyptol, myrcene, terpineol, limonene, caryophyllene, borneol and combinations thereof, and the side effect comprises fatigue, weakness, dizziness and/or deficits in balance. According to an embodiment, the primary terpene is selected from the group consisting of camphene, limonene, pinene, borneol and combinations thereof, and the side effect comprises palpitation and/or tachycardia. According to an embodiment, the primary terpene is selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof, and the side effect comprises digestive disorders including vomiting and/or diarrhea. According to an embodiment, the primary terpene is selected from the group consisting myrcene, linalool, caryophyllene, eucalyptol, limonene, nerolidol, fenchol, humulene, guaiol, borneol, bisabolol and combinations thereof, and the side effect comprises headache. According to an embodiment, the primary terpene is selected from the group consisting of pinene, eucalyptol, limonene and combinations thereof, and the side effect comprises coughing. According to an embodiment, the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial and combinations thereof, and the side effect comprises impaired fertility. According to an embodiment, the primary terpene is selected from the group consisting of eucalyptol, caryophyllene, limonene, humulene, phytol and combinations thereof, and the side effect comprises increased appetite. According to an embodiment, the primary terpene is selected from the group consisting of pinene, pulegone, eucalyptol, terpineol, cymene, ocimene, limonene, linalool, caryophyllene, humulene, carene and combinations thereof, and the side effect comprises impaired memory, impaired concentration and/or impaired perception. According to an embodiment, the primary terpene is selected from the group consisting of limonene, linalool, caryophyllene, eucalyptol, pinene, myrcene, terpinene, nerolidol, terpineol, geraniol, menthol, citronellol, neral, geraniol, menthol, borneol, phellandrene, bisabolol, fenchol, humulene and combinations thereof, and the side effect comprises anxiety and/or psychotic-like effects.

According to an embodiment, the product comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the product comprises a surfactant selected from the group consisting of phospholipids, glycerides, glycolipids and combinations thereof. According to an embodiment, the product further comprises a food-approved texturizer. According to an embodiment, the product further comprises at least 10 ppm ethanol. According to an embodiment, the product further comprises at least one of vitamin C, vitamin E, polyunsaturated fatty acids, beeswax and coconut oil. According to an embodiment, the product further comprises a sweetener. According to an embodiment, the product further comprises omega 3 fatty acid. According to an embodiment, the product further comprises omega 6 fatty acid. According to an embodiment, the product further comprises curcumin.

According to an embodiment, the terpene-enriched cannabinoid product of improved therapeutic effect is a human medication. According to an embodiment, the terpene-enriched cannabinoid product of improved therapeutic effect is a veterinary medication.

According to an embodiment, the shelf life of the product is at least 6 months or at least a year. According to an embodiment, primary terpene degradation in the product is less than 20% per year.

According to an embodiment, further provided is a product selected from the group consisting of tablets, gel capsules, medical patches, topicals, creams, varnishes, sublingual oils, sprays, edibles, beverages, suppositories, tampons, rectal candle, cigarette, vaporizer liquid nasal preparation, preparations containing micro and nano-emulsions, preparations containing micro and nano-particles and combinations thereof, containing the product.

According to an embodiment, further provided is a commercial product comprising two products according to the present invention, which two products differ in the content of the primary terpene. According to an embodiment, further provided is a commercial product comprising two products according to the present invention, which two products comprise different primary terpenes.

According to an embodiment, further provided is a method for producing the product comprising providing at least one selected cannabinoid and blending it with a primary terpene.

According to an embodiment, the method includes extracting Cannabis plant material to form an extract. According to an embodiment the extracting comprises steam distillation. According to an embodiment, the method further comprises removing terpenes from the extract prior to the blending. According to an embodiment, the extracting comprises contacting with an extractant to form an extract, which extract comprises at least one cannabinoid and the extractant; and optionally removing at least a fraction of the extractant from the extract. According to an embodiment, the extractant comprises at least one of ethanol, a liquefied gas, such as butane, butene or dimethyl-ether, liquefied CO₂, near-critical CO₂ supercritical CO₂ and combinations thereof. According to an embodiment, extracting comprises contacting the Cannabis plant material with an extractant to form an extract, and the extractant comprises at least one terpene. According to an embodiment, the extractant comprises the primary terpene.

According to an embodiment, the method further comprises at least partially decarboxylating the cannabinoid. According to an embodiment the decarboxylating is conducted at a temperature greater than 100° C. According to an embodiment the decarboxylating is conducted prior to extracting. According to an embodiment the decarboxylating is conducted on the extract prior to the blending with the primary terpene. According to an embodiment, a fraction of the extract is decarboxylated, and the decarboxylated fraction is blended with another fraction, which was not decarboxylated.

According to an embodiment, the method further comprises extracting a plant material, whereby the primary terpene is produced. According to an embodiment, the plant material is selected from the group including Cannabis, lemons, oranges, hops, lavender, pine needles, Echinacea, tea, clover, Capsicum, eucalyptus, geranium and mint.

According to an embodiment, the method further comprises blending Cannabis plant material with the primary terpene. According to an embodiment, the Cannabis plant material comprises a Cannabis bud. According to an embodiment, the blending Cannabis plant material with the primary terpene comprises spraying the Cannabis plant material with the primary terpene, optionally in a solvent. According to an embodiment, the blending Cannabis plant material with the primary terpene comprises combining the Cannabis plant material with a substrate comprising the primary terpene. According to an embodiment, the substrate is a plant material sprayed with the primary terpene. According to an embodiment, the substrate is a cigarette paper sprayed with the primary terpene.

According to an embodiment, further provided is a method for producing a product as disclosed herein, the method comprising providing at least one isolated or synthetic cannabinoid and blending it with a primary terpene.

According to an embodiment, further provided is a method for producing the product comprising extracting Cannabis plant material to form an extract, wherein the extracting forms at least two extract fractions, a cannabinoid-enriched extract and a terpene-enriched extract. As used herein, the term “cannabinoid-enriched extract” refers to an extract wherein cannabinoid to terpenes weight/weight ratio is greater than that in the plant material. As used herein, the term “terpene-enriched extract” refers to an extract wherein cannabinoid to terpenes weight/weight ratio is smaller than that in the plant material. According to an embodiment, the method comprises dividing the cannabinoid-enriched extract into at least two fractions and mixing at least one fraction with at least part of the terpene-enriched extract to form a terpene-enriched product, wherein terpenes to cannabinoid weight/weight ratio is greater than that ratio in the Cannabis plant material. According to an embodiment the ratio is 1.5 times greater than that in the Cannabis plant material, 2 times greater, 2.5 times greater, 3 times greater, 4 times greater, 5 times greater, 6 times greater, 7 times greater, 8 times greater, or 10 times greater. According to an embodiment, the terpene-enriched product is further mixed with at least one terpene.

According to an embodiment, the Cannabis extract, comprises at least one terpene, is blended with said selected cannabinoid.

According to an embodiment, the method includes contacting hemp plant material or a Cannabis plant material with an extractant to form an extract and isolating at least a fraction of the CBD from said extract. According to an embodiment the extracting comprises steam distillation. According to an embodiment, the method further comprises removing terpenes from the extract prior to the blending. According to an embodiment, the extracting comprises contacting with an extractant to form an extract, which extract comprises at least one cannabinoid and the extractant; and optionally removing at least a fraction of the extractant from the extract. According to an embodiment, the extractant comprises at least one of ethanol, a liquefied gas, such as butane, butene or dimethyl-ether, liquefied CO₂, near-critical CO₂ supercritical CO₂ and combinations thereof. According to an embodiment, extracting comprises contacting the hemp plant material with an extractant to form an extract, and the extractant comprises at least one terpene. According to an embodiment, the extractant comprises the primary terpene. According to an embodiment, the hemp and/or Cannabis extract is blended with said selected cannabinoid.

According to an embodiment, the method includes contacting Cannabis plant material with an extractant to form an extract, isolating at least a fraction of a cannabinoid from said extract, whereby terpenes to cannabinoids weight/weight ratio in said extract increases, and blending the separated cannabinoid with a primary terpene.

According to an embodiment, the method comprises synthesizing at least one selected cannabinoid, wherein said synthetizing comprise at least one chemical reaction, at least one biological reaction or a combination. According to an embodiment, the at least one selected cannabinoid is blended with the primary terpene.

According to an embodiment, said separated cannabinoid is crystallized.

According to an embodiment, said crystallized cannabinoid is diluted using an extractant.

According to an embodiment, said selected cannabinoid is selected from the group consisting of THC, CBD, CBC, CBG, CBN, CBL, THCV and/or CBDV.

According to an embodiment, further provided is a method of treatment comprising administering to a subject in need thereof a therapeutically effective amount of a product comprising (i) at least one isolated or synthetic cannabinoid, wherein at least a fraction of said isolated or synthetic cannabinoid is obtained by a process selected from the group consisting of processing hemp, chemical synthesis, biological synthesis, isolation from plant source and combination thereof, (ii) optionally, at least one other cannabinoid, (iii) a primary terpene, and (iv) optionally at least three secondary terpenes. According to some such embodiments, the product provides an enhanced therapeutic effect compared with that obtained by administrating a product comprising the same cannabinoids amount and no more than half the amount of terpene. According to some such embodiments, the product provides an enhanced therapeutic effect compared with that obtained by administrating a product comprising the same cannabinoids amount and no terpene.

According to an aspect of some embodiments of the present invention, there is provided a composition comprising (i) at least one isolated or synthetic cannabinoid, wherein at least a fraction of said isolated or synthetic cannabinoid is obtained by a process selected from the group consisting of processing hemp, chemical synthesis, biological synthesis, isolation from plant source and combination thereof, (ii) optionally, at least one other cannabinoid, (iii) a primary terpene, and (iv) optionally at least three secondary terpenes for use in therapy. In some such embodiments, therapy comprises treatment of a condition selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof. In some such embodiments, the composition provides an enhanced therapeutic effect compared with that obtained by administrating a product comprising the same cannabinoids amount and no terpene.

According to an aspect of some embodiments of the present invention, there is provided the use of a composition comprising (i) at least one isolated or synthetic cannabinoid, wherein at least a fraction of said isolated or synthetic cannabinoid is obtained by a process selected from the group consisting of processing hemp, chemical synthesis, biological synthesis, isolation from plant source and combination thereof, (ii) optionally, at least one other cannabinoid, (iii) a primary terpene, and (iv) optionally at least three secondary terpenes, for treatment of a condition selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

According to an embodiment, the method of treatment, composition for use or use provides an enhanced therapeutic effect compared with that obtained by administrating a product or composition comprising the same cannabinoids amount and one half the amount of said primary terpene. According to a related embodiment, the treatment method has an enhanced therapeutic effect compared with that obtained by administrating a product comprising the same cannabinoids amount and no Cannabis terpenes. According to a related embodiment, the treatment method has an enhanced therapeutic effect compared with that obtained by administrating a product comprising the same cannabinoids amount and 0.01% primary terpene.

According to an embodiment, the therapeutic product comprises at least two, at least three or at least four selected cannabinoids, wherein at least a fraction of said selected cannabinoids is derived from processing hemp, chemical synthesis, biological synthesis, isolation from plant source and combination thereof.

According to an embodiment, the primary terpene to cannabinoid weight/weight ratio in said administrated product is greater than 0.05.

According to an embodiment, a terpene to cannabinoid weight/weight ratio is greater than 0.05.

According to an embodiment, the administrated product provides an enhanced therapeutic effect in treating and/or healing and/or relieving and/or decreasing, preventing and/or reducing the risk associated with, and/or improving the quality of life associated with conditions and/or symptoms associated with at least one of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof. According to an embodiment, the conditions and/or symptoms are associated with aging.

According to an embodiment of the method, treating comprises treating conditions and/or symptoms associated with aging. According to a related embodiment, the primary terpene is selected from the group consisting of caryophyllene, linalool, pinene, eucalyptol, sabinene, terpineol, bisabolol, humulene, myrcene, nerolidol and combinations thereof. According to an embodiment, the composition comprises at least 2 of the terpenes, at least 3, at least 4 or at least 5 of the terpenes. According to an embodiment, the primary terpene is selected from the group consisting of caryophyllene, linalool, pinene, terpineol, humulene, bisabolol, guaiol and combinations thereof. According to an embodiment, the primary terpene is selected from the group consisting of caryophyllene, linalool, sabinene and combinations thereof.

According to an embodiment, the enhanced therapeutic effect applies to a child. As used herein the term “therapeutic effect applies to a child” refers to therapeutic effect observed when the treated patient is a child.

According to an embodiment, the administrated product has an enhanced therapeutic effect in treating a child. According to a related embodiment, the primary terpene is selected from the group consisting of caryophyllene, limonene, pinene, terpineol, humulene, bisabolol, guaiol and combinations thereof. According to an embodiment, the composition comprises at least 2 of the terpenes, at least 3, or all 4 of the terpenes. According to an embodiment, the primary terpene is selected from the group consisting of caryophyllene, limonene and combination thereof.

According to an embodiment, the conditions and/or symptoms are observed in a child.

According to an embodiment, the product is selected from the group consisting of cigarettes, vaporizer plant material, vaporizer liquid, extract, tablets, gel capsules, suppositories, tampons, rectal candle, cigarette, vaporizer liquid, nasal preparation, preparations containing micro and nano-emulsions, preparations containing micro and nano-particles and combinations thereof. According to an embodiment, the daily dose of the product comprises about 1 milligram cannabinoid to about 300 milligram, about 2 milligram cannabinoid to about 200 milligram or about 3 milligram cannabinoid to about 100 milligram. According to another embodiment, the daily dose of the product comprises about 0.5 milligram cannabinoid per kilogram body weight to about 30 milligram cannabinoid per kilogram body weight.

According to an embodiment, the administered product comprises at least two cannabinoids, at least three, at least four or at least five. According to an embodiment, the content of each cannabinoid in the product is at least 10 parts per million (ppm). According to an embodiment, the cannabinoids are selected from the group consisting THC, CBD, CBG, CBC, CBN, CBL, THCV, CBDV and their non-decarboxylated form thereof.

According to an embodiment, the selected cannabinoids are selected from the group consisting of tetrahydrocannabiniol (THC), cannabidiol (CBD), cannabigerol (CBG), cannabichromene (CBC), tetrahydrocannabivarin (THCV), Cannabidivarin (CBDV), Cannabinol (CBN), Cannabicyclol (CBL).

According to an embodiment, at least a fraction of the selected cannabinoids is derived from processing hemp, chemical synthesis, biological synthesis, isolation from plant source and combination thereof.

According to an embodiment, the product comprises at least 5% by weight of a non-cannabinoid, non-terpene carrier.

According to an embodiment, the product comprises at least 2% by weight carrier, at least 3%, at least 5%, at least 7%, at least 10%, at least 12%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof.

According to an embodiment, said product comprises less than 1% THC.

According to an embodiment, the administered product comprises a primary terpene and optionally at least one secondary terpenes, at least two, at least three secondary terpenes, at least four, or at least five secondary terpenes. As used herein, the term “primary terpene” refers to a terpene that forms at least 20% by weight of the total amount of terpenes in the product, at least 25%, at least 30%, at least 35%, at least 40%, at least 50%, at least 60%, at least 70% or at least 80%. As used herein, the term “primary terpene” refers to a terpene having the greatest amount in the composition. As used herein, the term “primary terpene” refers to a terpene that forms the highest terpene concentration in the composition. According to an embodiment, the product comprises multiple (e.g. two, three, or four) According to an embodiment, the primary terpene forms at least 20% by weight of the total terpene content, at least 30%, at least 40%, at least 50%, at least 60%, at least 70% or at least 80%, at least 90% by weight of the total terpene content. According to an embodiment, the product comprises multiple (e.g. two or three) terpenes, each one of which forms at least 20% by weight of the total amount of terpenes in the product and each one of these terpenes is considered a primary terpene. According to an embodiment, the product comprises multiple (e.g., two, three, or four) terpenes, each one of which forms at least 15% by weight of the total amount of terpenes in the product, at least 18%, at least 22%, at least 25%, at least 30%, at least 35%, or at least 40%, and each one of these terpenes is considered a primary terpene. As used here, in the term “secondary terpene” refers to a terpene that forms at least 10 parts per million (ppm) of the product. According to an embodiment, the content of the primary terpene in the product is at least 1.3 times greater than that of any secondary terpene, at least 1.5, at least 2, at least 2.2, at least 2.5, at least 3, at least 3.5, at least 4, at least 6, at least 8, at least 10, at least 15, at least 20, at least 25, or at least 30 times greater.

According to an embodiment, the non-cannabinoid, non-terpene carrier comprises cellulose and the terpenes to cannabinoids weight/weight ratio in the product is about 0.1 to about 1.0. According to an embodiment said non-cannabinoid, non-terpene, carrier comprises at least 5% by weight cellulose, at least 6%, at least 8%, at least 10%, at least 15%, or at least 20%. According to an embodiment, the ratio is greater than 0.1, greater than 0.12, greater than 0.13, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 1, less than 0.9, less than 0.8, less than 0.7, less than 0.6, less than 0.5, less than 0.4, less than 0.3, less than 0.2 or less than 0.15. According to an embodiment, the product comprising more than 5% cellulose is selected from the group consisting of Cannabis plant material, e.g. Cannabis buds (also referred to as Cannabis inflorescence) or Cannabis trim, ground forms thereof, plant material preparation for vaporizers and Cannabis cigarette. According to an embodiment, the product comprises a dried Cannabis plant material.

According to an embodiment, the non-cannabinoid, non-terpene carrier comprises less than 5% by weight cellulose and the terpenes to cannabinoids weight/weight ratio in the product is about 0.05 to about 1.0. According to an embodiment said non-cannabinoid, non-terpene, carrier comprises less than 5% by weight cellulose, less than 4%, less than 3%, less than 2%, or less than 1% cellulose. According to an embodiment, the ratio is greater than 0.05, greater than 0.06, greater than 0.07, greater than 0.08, greater than 0.09, greater than 0.1, greater than 0.12, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 1, less than 0.9, less than 0.8, less than 0.7, less than 0.6, less than 0.5, less than 0.4, less than 0.3, less than 0.2, less than 0.15, less than 0.1, less than 0.09, less than 0.08, less than 0.07, less than 0.06, or less than 0.05. According to an embodiment, the product comprising less than 5% cellulose is selected from the group consisting of Cannabis trichomes, Cannabis extracts and products thereof, such as tablets, gel capsules, medical patches, topicals, creams, varnishes, sublingual oils, sprays, edibles, beverages, suppositories, tampons, rectal candles, cigarettes, vaporizer liquids, nasal preparation, preparations containing micro and nano-emulsions, preparations containing micro and nano-particles and combinations thereof. According to an embodiment, said product comprises a liquid.

According to an embodiment, the primary terpene, the secondary terpenes or both are selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol, isomers thereof and combinations thereof. According to an embodiment, the primary terpene, the secondary terpenes or both, are selected from the group consisting of pinene, limonene, linalool, caryophyllene, myrcene, terpineol, eucalyptol, geraniol and combinations thereof. According to an embodiment, the primary terpene the secondary terpenes or both, are selected from the group consisting of pinene, limonene, linalool, caryophyllene, myrcene, terpineol, eucalyptol and combinations thereof. According to an embodiment, the primary terpene is selected from the group consisting of pinene, limonene, linalool, caryophyllene, myrcene, terpineol, eucalyptol, geraniol, humulene and combinations thereof. According to an embodiment, at least one of the terpenes is a-cyclic. According to an embodiment, at least one of the terpenes is cyclic. According to an embodiment, at least one of the terpenes is not found in Cannabis buds or is present there at less than 0.2%, less than 0.1%, less than 0.05% or less than 0.02%. Such terpene is referred to as “non-Cannabis terpene”. According to an embodiment, the terpene-enriched cannabinoid product comprises the non-Cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, terpenes form at least 0.5% by weight of the product, at least 0.6%, at least 0.8%, at least 1%, at least 2%, at least 3%, at least 5%, at least 10%, at least 20%, at least 30%, at least 40%, at least 50%, at least 60% or at least 70%.

According to an embodiment, the product comprises a composition, which is liquid at 30° C. According to an embodiment, the product comprises a composition, which is a suspension at 30° C. According to an embodiment, the product comprises a composition, which is essentially clear of haze or suspended solids at 30° C.

According to an embodiment, the product comprises Cannabis plant material. According to an embodiment, the product comprises Cannabis bud. According to an embodiment, the Cannabis bud forms at least 20% of the product, at least 30%, at least 40%, at least 50%, at least 60%, at least 70% or at least 80%.

According to an embodiment, the administered product is combines with at least one herbal extract, at least two, at least three, at least four, or at least five herbal extracts.

According to an embodiment, the administered product comprises less than 5% by weight glycol, less than 4%, less than 3%, less than 2%, or less than 1% by weight.

According to an embodiment, the administered product comprises less than 20% by weight water, less than 15% by weight, less than 14%, less than 13%, less than 12%, less than 11% or less than 10% by weight.

According to a related embodiment, the administered product comprises chlorophyll. According to an embodiment, the product comprises at least 0.5% by weight chlorophyll, at least 1%, at least 5%, at least 10%, at least 15%, at least 20% chlorophyll. According to an embodiment, the administered product comprises at least one flavonoid. According to an embodiment, the product comprises at least two, at least three, at least four, or at least five flavonoids. According to an embodiment, the product comprises at least one of bergamottin, apigenin, amentoflavone, quercetin and piperine. According to an embodiment, the product comprises at least two, at least three, or at least four of bergamottin, apigenin, amentoflavone, quercetin and piperine. According to an embodiment, the product comprises bergamottin. According to an embodiment, the product comprises apigenin. According to an embodiment, the product comprises amentoflavone. According to an embodiment, the product comprises quercetin. According to an embodiment, the product comprises piperine.

According to an embodiment, the administered product additionally contains an additive selected from the group consisting of antioxidants, emulsifiers and texturizers vegetable oils, plant extracts, honey, sucrose, glucose and fructose, pharmaceutical excipients and combinations thereof.

According to an embodiment, the product comprises less than 0.5% chlorophyll.

According to an embodiment, the product comprises less than 0.5% cellulose.

According to an embodiment, said isolated or synthetic cannabinoid is obtained by biological synthesis, wherein said product comprises a biological remnant derived from said biological synthesis.

According to an embodiment, said isolated or synthetic cannabinoid is an isolated cannabinoid.

According to an embodiment, said isolated or synthetic cannabinoid is crystallized.

According to an embodiment, the concentration of said isolated or synthetic cannabinoid in said product is lower than 50%, lower than 40%, lower than 30%, lower than 20% or lower than 10%.

According to an embodiment the product comprises tetrahydrocannabinol (THC) and/or tetrahydrocannabinolic acid (THCa), wherein THC and/or THCa is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises cannabidiol (CBD) and/or cannabidiolic acid (CBDa), wherein CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises tetrahydrocannabinol (THC) and/or tetrahydrocannabinolic acid (THCa) and cannabidiol (CBD) and/or cannabidiolic acid (CBDa), wherein, THC and/or THCa is in a total concentration of at least 2.5% by weight, and CBD and/or CBDa is in a total concentration of at least 2.5% by weight; THC and/or THCa in a total concentration of at least 3% by weight, and CBD and/or CBDa in a total concentration of at least 3% by weight; THC and/or THCa in a total concentration of at least 4% by weight, and CBD and/or CBDa in a total concentration of at least 4% by weight; THC and/or THCa in a total concentration of at least 5% by weight, and CBD and/or CBDa in a total concentration of at least 5% by weight; THC and/or THCa in a total concentration of at least 8% by weight, and CBD and/or CBDa in a total concentration of at least 8% by weight; THC and/or THCa in a total concentration of at least 10% by weight, and c CBD and/or CBDa in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment the product comprises selected tetrahydrocannabinol (THC) and, wherein selected THC is in a total concentration of at least 1% by weight, at least 2%, at least 3%, at least 4%, at least 5%, at least 6%, at least 8%, at least 10%, at least 12%, at least 14%, at least 16%, at least 18%, or at least 20%, at least 30%, at least 40%, at least 50%, at least 60%, at least 70%, at least 80%, or at least 90% by weight.

According to an embodiment, the product comprises selected cannabidiol (CBD), wherein selected CBD in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product comprises selected tetrahydrocannabinol (THC) and selected cannabidiol (CBD), wherein, selected THC is in a total concentration of at least 2.5% by weight, and selected CBD is in a total concentration of at least 2.5% by weight; selected THC in a total concentration of at least 3% by weight, and selected CBD in a total concentration of at least 3% by weight; selected THC in a total concentration of at least 4% by weight, and selected CBD in a total concentration of at least 4% by weight; selected THC in a total concentration of at least 5% by weight, and selected CBD in a total concentration of at least 5% by weight; selected THC in a total concentration of at least 8% by weight, and selected CBD in a total concentration of at least 8% by weight; selected THC in a total concentration of at least 10% by weight, and selected CBD in a total concentration of at least 10% by weight; selected THC in a total concentration of at least 20% by weight, and selected CBD in a total concentration of at least 20% by weight; selected THC in a total concentration of at least 30% by weight, and selected CBD in a total concentration of at least 30% by weight.

According to a related embodiment, said product comprises selected cannabigerol (CBG) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabinol (CBN) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabichromene (CBC) in a total concentration of at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabicyclol (CBL) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected tetrahydrocannabivarin (THCV) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment, said product comprises selected cannabidivarin (CBDV) in a total concentration at least 0.1% by weight, at least 0.5%, at least 1%, at least 2%, at least 3%, at least 4%, or at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to an embodiment, the product results in an increased and/or enhanced therapeutic effect compared with that of a product comprising the same amount of cannabinoid and optionally secondary terpenes and no terpene. According to an embodiment, the product results in an increased and/or enhanced therapeutic effect compared with that of a product comprising the same amounts of cannabinoids and optionally secondary terpenes and a smaller amount of the primary terpene, e.g. one half of that amount. According to an embodiment, the product results in an increased and/or enhanced therapeutic effect compared with that of a product comprising the same amount of cannabinoid and no Cannabis terpenes. According to an embodiment, the product results in an increased and/or enhanced therapeutic effect compared with that of a product comprising the same amount of cannabinoid and 0.01% primary terpene. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, said enhanced therapeutic effect comprises shorter onset time of the therapeutic effect. According to an embodiment, the primary terpene, the cannabinoids and optionally secondary terpenes are present in specific amounts, and the onset time of the therapeutic effect, as measured by methods known in the art, is at least 20% shorter than that of a product comprising no terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% shorter than that of a product comprising the same cannabinoids amount and 0.01% primary terpene, at least 30%, at least 40%, at least 50%, at least 60%, or at least 70% shorter. Shorter onset time, or differently put an earlier effect, is important particularly in cases of sublingual, edible and topical delivery and in cases where a rapid effect is desired, as in treating pain.

According to an embodiment, said enhanced therapeutic effect comprises longer onset time of the therapeutic effect. According to an embodiment, the primary terpene, the cannabinoids and optionally secondary terpenes are present in specific amounts, and the onset time of the therapeutic effect, as measured by methods known in the art, is at least 20% longer than that of a product comprising the same amount of cannabinoid and no terpene, at least 30%, at least 40%, at least 50%, at least 60%, or at least 70% longer. According to an embodiment, the primary terpene, the cannabinoids and optionally secondary terpenes are present in specific amounts, and the onset time of the therapeutic effect, as measured by methods known in the art, is at least 20% longer than that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the onset time of the therapeutic effect, as measured by methods known in the art, is at least 20% longer than that of a product comprising the same cannabinoids amount and 0.01% primary terpene, at least 30%, at least 40%, at least 50%, at least 60%, or at least 70% shorter. According to an embodiment, products of delayed onset time are used in combination with shorter onset time to reach a sustained release effect.

According to an embodiment, said enhanced therapeutic effect comprises greater and/or increased magnitude of the therapeutic effect. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amount of cannabinoid and no terpene, at least 30%, at least 40%, at least 50%, at least 60%, or at least 70% greater. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the magnitude of the therapeutic effect, as measured by methods known in the art, is at least 20% greater compared with that of a product comprising the same cannabinoids amount and 0.01% primary terpene, at least 30%, at least 40%, at least 50%, at least 60%, or at least 70% shorter. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, said enhanced therapeutic effect comprises longer and/or extended duration of the therapeutic effect. According to an embodiment, the primary terpene, the cannabinoids and optionally secondary terpenes are present in specific amounts, and the duration of the therapeutic effect, as measured by methods known in the art, is at least 20% longer compared with that of a product comprising the same amount of cannabinoid and no terpene, at least 30%, at least 40%, at least 50%, at least 60%, at least 70%, at least 80%, at least 90% or at least 100% longer. According to an embodiment, the primary terpene, the cannabinoids and optionally secondary terpenes are present in specific amounts, and the duration of the therapeutic effect, as measured by methods known in the art, is at least 20% longer compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the duration of the therapeutic effect, as measured by methods known in the art, is at least 20% longer compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises smaller therapeutically effective amount of a product and/or reduced dosages of the product. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amount of cannabinoid and no terpene, at least 30%, at least 40%, at least 50%, at least 60%, at least 70%, at least 80%, at least 90% or at least 100% smaller. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, and the therapeutically effective amount of the product, as measured by methods known in the art, is at least 20% smaller compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises reduced secondary adverse symptoms. According to an embodiment, the primary terpene, the cannabinoids and optionally secondary terpenes are present in specific amounts, and the secondary adverse symptoms, as measured by methods known in the art, are reduced by at least 20% compared with that of a product comprising the same amount of cannabinoid and no terpene, by at least 30%, at least 40%, at least 50%, at least 60%, at least 70%, at least 80%, at least 90% or at least 100%. According to an embodiment, the primary terpene, the cannabinoids and optionally secondary terpenes are present in specific amounts, and secondary adverse symptoms, as measured by methods known in the art, are reduced by at least 20% compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and secondary adverse symptoms, as measured by methods known in the art, are reduced by at least 20% compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and secondary adverse symptoms, as measured by methods known in the art, are reduced by at least 20% compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, secondary adverse symptoms, as measured by methods known in the art, are reduced by at least 20% compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises smaller and/or reduced frequency of conditions and/or symptoms. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amount of cannabinoid and no terpene, at least 30%, at least 40%, at least 50% at least 60%, at least 70%, at least 80%, or at least 90% smaller. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the frequency of the conditions and/or symptoms, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the frequency of the conditions and/or symptoms, as measured by methods known in the art, is at least 20% smaller compared with that of a product comprising the same cannabinoids amount and 0.01% primary terpene, at least 30%, at least 40%, at least 50%, at least 60%, or at least 70% shorter. According to an embodiment, said enhanced therapeutic effect comprises smaller and/or reduced severity of conditions and/or symptoms. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amounts of cannabinoids and no terpene terpene, at least 30%, at least 40%, at least 50% at least 60%, at least 70%, at least 80%, or at least 90% smaller. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the severity of the conditions and/or symptoms, as measured by methods known in the art, is at least 20% smaller compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the severity of the conditions and/or symptoms, as measured by methods known in the art, is at least 20% smaller compared with that of a product comprising the same cannabinoids amount and 0.01% primary terpene, at least 30%, at least 40%, at least 50%, at least 60%, or at least 70% shorter.

According to an embodiment, said enhanced therapeutic effect comprises smaller and/or reduced consumption of other drugs. According to an embodiment, the primary terpene, the cannabinoids and optionally secondary terpenes are present in specific amounts, and the consumption of other drugs is reduced by at least 20% compared with that of a product comprising the same amount of cannabinoid and no terpene, reduced by at least 30%, by at least 40%, by at least 50%, by at least 60%, by at least 70%, by at least 80%, or by at least 90%. According to an embodiment, the primary terpene, the cannabinoids and optionally secondary terpenes are present in specific amounts, and the consumption of other drugs is reduced by at least 20% compared with that of a product comprising the same amount of cannabinoid and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the consumption of other drugs is reduced by at least 20% compared with that of a product comprising the same amounts of cannabinoids and secondary terpenes and one half the amount of the primary terpene, at least 30%, at least 40%, at least 50%, at least 60%, or at least 70% shorter. According to an embodiment, the primary terpene, the cannabinoids and optionally secondary terpenes are present in specific amounts, and the consumption of other drugs is reduced by at least 20% compared with that of a product comprising the same cannabinoids amount and no Cannabis terpenes, at least 30%, at least 40%, at least 50%, at least 60%, or at least 70% shorter. According to an embodiment, the primary terpene, the cannabinoids and optionally secondary terpenes are present in specific amounts, the consumption of other drugs is reduced by at least 20% compared with that of a product comprising the same cannabinoids amount and 0.01% primary terpene, at least 30%, at least 40%, at least 50%, at least 60%, or at least 70% shorter.

According to an embodiment, the administrated product comprises tetrahydrocannabinol (THC) at a concentration of less than 5% by weight, and at least 0.5% by weight primary terpene. According to an embodiment, the product is applied to a child. According to an embodiment, the primary terpene is selected from the group consisting of limonene, linalool, eucalyptol, myrcene, caryophyllene, terpineol, bisabolol, pinene and combinations thereof. According to an embodiment, the composition comprises tetrahydrocannabinol (THC) in a concentration of less than 5%, less than 4%, less than 3%, less than 2%, or less than 1%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of limonene, linalool, myrcene and combination thereof.

According to an embodiment, the administrated product comprises CBD and optionally THC at CBD to THC weight/weight ratio greater than 1, wherein CBD concentration is less than 20% by weight and wherein primary terpene concentration is at least 0.5% by weight. According to an embodiment, the product is applied to a child. According to an embodiment, the primary terpene is selected from the group consisting of linalool, terpineol, ocimene, humulene, caryophyllene, eucalyptol, pinene, limonene, bisabol, guaiol and combinations thereof. According to an embodiment, the product comprises CBD and optionally THC at CBD to THC weight/weight ratio greater 2, greater than 3, greater than 4 or greater than 5. According to an embodiment, the composition comprises cannabidiol (CBD) and/or cannabidiolic acid (CBDa) in a concentration of less than 18%, less than 16%, less than 14%, less than 12%, or less than 10%. According to an embodiment, the composition comprises cannabidiol (CBD) and/or cannabidiolic acid (CBDa), wherein CBD and/or CBDa concentration is of 10 to 20%, 10 to 15%, 15 to 20%, 12-18%. According to an embodiment, the primary terpene is in a concentration of at least 0.5%, at least 0.6%, at least 0.7%, at least 0.8%, at least 0.9%, or at least 1%. According to an embodiment, the composition comprises at least 2 of said primary terpenes, at least 3, at least 4, or at least 5. According to an embodiment, the primary terpene is selected from the group consisting of linalool, terpineol, caryophyllene and combination thereof. According to an embodiment, the primary terpene is selected from the group consisting of linalool, terpineol, ocimene and combination thereof. According to an embodiment, the primary terpene is selected from the group consisting of eucalyptol, pinene, limonene and combination thereof. According to an embodiment, the primary terpene comprises caryophyllene and/or humulene.

According to an embodiment, the therapeutic effect is generated while administering the product in a vaporizer. According to an embodiment, the therapeutic effect is generated while administering the product by inhaling. According to an embodiment, the therapeutic effect is generated while administering the product by smoking. According to an embodiment, the therapeutic effect is generated while administering the product sublingually. According to an embodiment, the therapeutic effect is generated while applying the product, topically. According to an embodiment, the therapeutic effect is generated while administering the product in suppositories, including tampons and/or rectal candles. According to an embodiment, the therapeutic effect is generated while administering the product in a spray. According to an embodiment, the therapeutic effect is generated while administering the product as an edible, as a candy, as baked good, as a drink. According to an embodiment, the therapeutic effect is generated while administering the product as vaporizer liquid nasal preparation. According to an embodiment, the therapeutic effect is generated while administering the product as micro and nano-emulsions. According to an embodiment, the therapeutic effect is generated while administering the product as micro and nano-emulsions.

According to an embodiment, said administrated product further comprising a herbal preparation, wherein said herbal preparation is produced by at least one of distilling and extracting.

According to an embodiment, the administrated product comprises herbal preparation selected from the group consisting of extracts of Acorus, Acorus calamus, Aloe, Ammi visnaga, Angelica arc, Angelica archangelica Radix, Angelica sinensis, Artemisia, Asphalentum ponjabianum-mumio, Astragalus, Atractylodes macrocephala, Avena sativa, Bacopa, Bacopa monnieri, Barosma betulina, Boswellia, Boswellia spp, Capsicum spp, Caulophyllum thalictroides, Centella, Cimicfuga, Cimicifuga, Cimicifuga (Actaea) racemose, Cinnamomum zeylanicum, Coptis chinensis, Corydalis yanhusuo, Crataegus spp, Crataegus spp. Curcuma, Curcuma longa, Dioscorea villosa, Dioscorea Villosa spp, Echinacea spp, Eleutherococcus senticosus, Entella (Hydrocotyl) asiatica, Filipendula ulmaria, Foeniculum, Foeniculum vulgare, Fucus vesiculosus, Galium, Galium, Ganoderma, Ganoderma lucidum, Garcinia, Ginco, Ginco Biloba, Ginseng, Glycyrrhiza glabra, Grifola, Grifola frondosa, Gymnema, Harpagophytum procumbens, Humulus, Humulus lupulus, Hydrastis, Hydrastis canadensis, Hypericum, Hypericum perforatum, Iris versicolor, Laminaria, Lavendula officinalis, Lentinula edodes, Leonarous, Leonurus spp, Lepidium meyenii, Lepidium meyenii, Lycium, Lycium barbarum, Matricaria, Melissa officinalis, Mentha piperita, Mitaki, Mumio, Olea europea, origanum, Paeonia lactiflora, Panax ginseng, Passiflora, Passiflora incarnata, Paullinia cupana, Paullinia cupana, Peonia, Peumus boldus, Phelodendron amurense, Piscidia, Piscidia erythrina, Rehmania glutinosa, Preparata, Rhamnus purshiana, Rhodiola, Rhodiola rosea, Rosmarinus officinalis, Rozmarinous, Rumex, Salix, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum spp. Radix, Tribulus terrestris, Trifolium pretense, Trigonella foenum graecum, Turnera, Uncaria tomentosa, Valeriana officinalis, Valleriane, Verbena officinalis, vibronum, Viburnum opulus, Viburnum prunifolium, Viscum Albumvitex, Vitex agnus castus, Witania, Zanthoxylum spp, Zingiber officinalis and combinations thereof.

According to an embodiment said therapeutic effect treats diabetes and said administered product comprises at least one of CBD, THC, CBC, CBG, CBN, CBL, THCV and CBDV. According to an embodiment said therapeutic effect treats diabetes and said product comprises CBD and optionally THC at CBD to THC weight/weight ratio greater than 1, greater than 2, greater than 3, greater than 4 or greater than 5. According to an embodiment said therapeutic effect treats diabetes and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats diabetes and said primary terpene is selected from the group consisting of limonene, pinene, linalool, humulene, myrcene, phytol, geraniol, bisabolol, borneol, guaiol, ocimene, fenchol, terpineol, terpinene, eucalyptol and combinations thereof. According to an embodiment, the product comprises at least 2 of these terpenes, at least 3, at least 4, or at least 5 of the terpenes. According to an embodiment, the primary terpene is selected from the group consisting of limonene, linalool, geraniol and combinations thereof. According to an embodiment, the primary terpene is selected from the group consisting of limonene, linalool, geraniol, pinene, humulene, phytol and combinations thereof. According to various embodiments said primary terpene comprises limonene, pinene, linalool, humulene, myrcene, phytol, geraniol, bisabolol, borneol, guaiol, ocimene, fenchol, terpineol, terpinene and/or eucalyptol.

According to an embodiment said therapeutic effect treats including Crohn's disease and ulcerative colitis and said administered product comprises at least one of CBD, THC, CBC, CBG, CBN, CBL, THCV and CBDV. According to an embodiment said therapeutic effect treats including Crohn's disease and ulcerative colitis and said product comprises CBD and optionally THC at CBD to THC weight/weight ratio greater than 1, greater than 2, greater than 3, greater than 4 or greater than 5. According to an embodiment said therapeutic effect treats including Crohn's disease and ulcerative colitis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats including Crohn's disease and ulcerative colitis and said primary terpene is selected from the group consisting of limonene, caryophyllene, borneol, myrcene, pinene, terpinene, isopulegol, phellandrene, carene, carvacol, terpineol, humulene, linalool, nerolidol, guaiol and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, myrcene and combinations thereof. According to an embodiment, the primary terpene is selected from the group consisting of limonene, caryophyllene, myrcene, pinene, carene, carvacol and combinations thereof. According to various embodiments said primary terpene comprises limonene, caryophyllene, borneol, myrcene, pinene, terpinene, isopulegol, phellandrene, carene, carvacol, terpineol and/or humulene. According to an embodiment, said therapeutic effect treats Crohn's disease and ulcerative colitis and the product further comprises a compound selected from the group consisting of linalyl acetate, geranyl acetate, eugenol, eugenyl acetate, piperine and combinations thereof. According to an embodiment, the product includes fenchol and fenchol forms less than 5% by weight of the total terpene content.

According to an embodiment said therapeutic effect stimulates the immune system and/or treats weakened immune system and said administered product comprises at least one of CBD, THC, CBC, CBG, CBN, CBL, THCV and CBDV. According to an embodiment said therapeutic effect stimulates the immune system and/or treats weakened immune system and said product comprises CBD and optionally THC at CBD to THC weight/weight ratio greater than 1, greater than 2, greater than 3, greater than 4 or greater than 5. According to an embodiment said therapeutic effect stimulates the immune system and/or treats weakened immune system and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect stimulates the immune system and/or treats weakened immune system and said primary terpene is selected from the group consisting linalool, pinene, borneol, caryophyllene, geraniol, amyrin, terpinene, myrcene, eucalyptol, humulene and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, amyrin, and combinations thereof. According to an embodiment, the primary terpene is selected from the group consisting of linalool, caryophyllene, amyrin, pinene, terpinene, myrcene and combinations thereof. According to various embodiments said primary terpene comprises linalool, pinene, borneol, caryophyllene, geraniol, amyrin, terpinene, myrcene, eucalyptol and/or humulene.

According to an embodiment said therapeutic effect treats Fibromyalgia and said administered product comprises at least one of CBD, THC, CBC, CBG, CBN, CBL, THCV and CBDV. According to an embodiment said therapeutic effect treats Fibromyalgia and said product comprises CBD and optionally THC at CBD to THC weight/weight ratio greater than 1, greater than 2, greater than 3, greater than 4 or greater than 5. According to an embodiment said therapeutic effect treats Fibromyalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats Fibromyalgia and said primary terpene is selected from the group consisting of linalool, caryophyllene, limonene, humulene, eucalyptol, amyrin, cycloartenol, pinene, sabinene, myrcene, terpinene, borneol, bisabolol, guaiol, terpineol, ocimene, camphene and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, eucalyptol and combinations thereof. According to an embodiment, the primary terpene is selected from the group consisting of linalool, caryophyllene, eucalyptol, limonene, pinene, sabinene, myrcene and combinations thereof. According to various embodiments said primary terpene comprises linalool, caryophyllene, limonene, humulene, eucalyptol, amyrin, cycloartenol, pinene, sabinene, myrcene, terpinene, borneol and/or bisabolol. According to an embodiment, the product includes myrcene, humulene, bisabolol, borneol, limonene and/or linalool and myrcene, humulene, bisabolol, borneol, limonene and/or linalool forms less than 5% by weight of the total terpene content.

According to an embodiment said therapeutic effect treats nociceptive pain and said administered product comprises at least one of CBD, THC, CBC, CBG, CBN, CBL, THCV and CBDV. According to an embodiment said therapeutic effect treats nociceptive pain and said product comprises CBD and optionally THC at CBD to THC weight/weight ratio greater than 1, greater than 2, greater than 3, greater than 4 or greater than 5. According to an embodiment said therapeutic effect treats nociceptive pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats nociceptive pain and said primary terpene is selected from the group consisting of myrcene, linalool, caryophyllene, eucalyptol, cycloartenol, borneol, sabinene, camphene, terpineol, citronellol, geraniol, humulene, bisabolol, limonene, pinene, guaiol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, eucalyptol, terpineol, citronellol and combinations thereof. According to various embodiments said primary terpene comprises myrcene, linalool, caryophyllene, eucalyptol, cycloartenol, borneol, sabinene, camphene, terpineol, citronellol, geraniol, humulene, bisabolol and/or limonene.

According to an embodiment said therapeutic effect treats neuropathic pain and said administered product comprises at least one of CBD, THC, CBC, CBG, CBN, CBL, THCV and CBDV. According to an embodiment said therapeutic effect treats neuropathic pain and said product comprises CBD and optionally THC at CBD to THC weight/weight ratio greater than 1, greater than 2, greater than 3, greater than 4 or greater than 5. According to an embodiment said therapeutic effect treats neuropathic pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats neuropathic pain and said primary terpene is selected from the group consisting of caryophyllene, linalool, eucalyptol, amyrin, borneol, pinene, terpineol, limonene, nerolidol, geraniol, myrcene, humulene, bisabolol, guaioland combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, eucalyptol and combinations thereof. According to an embodiment, the primary terpene is selected from the group consisting of caryophyllene, linalool, eucalyptol, borneol, limonene, myrcene and combinations thereof. According to an embodiment, the primary terpene is selected from the group consisting of pinene, terpineol, nerolidol and combinations thereof. According to various embodiments said primary terpene comprises caryophyllene, linalool, eucalyptol, amyrin, borneol, pinene, terpineol, limonene, nerolidol, geraniol and/or myrcene. According to an embodiment, the product includes humulene, bisabolol, borneol, limonene and/or linalool, and humulene, bisabolol, borneol, limonene and/or linalool forms less than 5% by weight of the total terpene content.

According to an embodiment said therapeutic effect treats pain and said product comprises at least one of CBD, THC, CBC, CBG, CBN, CBL, THCV and CBDV. According to an embodiment said therapeutic effect treats pain and said administered product comprises CBD and optionally THC at CBD to THC weight/weight ratio greater than 1, greater than 2, greater than 3, greater than 4 or greater than 5. According to an embodiment said therapeutic effect treats pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and said primary terpene is selected from the group consisting of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, eucalyptol, borneol, terpineol and combinations thereof. According to various embodiments said primary terpene comprises myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol and/or limonene. According to an embodiment, said therapeutic effect treats pain and the product comprises a compound selected from the group consisting of linalyl acetate, geranyl acetate, eugenol, eugenyl acetate, piperine, methylis-oeugenol, Methyleugenol, menthol and combination thereof. According to an embodiment, the product includes humulene, bisabolol, borneol, limonene and/or linalool, wherein humulene, bisabolol, borneol, limonene and/or linalool forms less than 5% by weight of the total terpene content. According to an embodiment, said product comprises a herbal extract selected from the group consisting of extracts of Angelica sinensis, Piscidia erythrina, Tanacetum parthenium, Viburnum opulus, Boswellia spp, Capsicum spp, Corydalis yanhusuo, Harpagophytum procumbens, Rosmarinus officinalis, Salix alba, Zingiber officinalis and combinations thereof.

According to an embodiment said therapeutic effect treats arm or leg pain and said administered product comprises at least one of CBD, THC, CBC, CBG, CBN, CBL, THCV and CBDV. According to an embodiment said therapeutic effect treats arm or leg pain and said product comprises CBD and optionally THC at CBD to THC weight/weight ratio greater than 1, greater than 2, greater than 3, greater than 4 or greater than 5. According to an embodiment said therapeutic effect treats arm or leg pain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats arm or leg pain and said primary terpene is selected from the group consisting of linalool, terpinene, pinene, limonene, terpineol, myrcene, nerolidol and combinations thereof. According to an embodiment, the product comprises at least 2 of these terpenes, at least 3, at least 4, or at least 5 of the terpenes. According to an embodiment, the primary terpene is selected from the group consisting of linalool, pinene, myrcene and combinations thereof. According to an embodiment, the primary terpene is selected from the group consisting of linalool, pinene, myrcene, limonene, terpineol, nerolidol and combinations thereof. According to various embodiments said primary terpene comprises linalool, terpinene, pinene, limonene, terpineol, myrcene and/or nerolidol.

According to an embodiment said therapeutic effect treats neurodegenerative diseases including Alzheimer disease, Parkinson disease, Huntington's disease, dystonia and/or Amyotrophic lateral sclerosis (ALS) and said administered product comprises at least one of CBD, THC, CBC, CBG, CBN, CBL, THCV and CBDV. According to an embodiment said therapeutic effect treats neurodegenerative diseases including Alzheimer disease, Parkinson disease, Huntington's disease, dystonia and/or Amyotrophic lateral sclerosis (ALS) and said product comprises CBD and optionally THC at CBD to THC weight/weight ratio greater than 1, greater than 2, greater than 3, greater than 4 or greater than 5. According to an embodiment said therapeutic effect treats neurodegenerative diseases including Alzheimer disease, Parkinson disease, Huntington's disease, dystonia and/or Amyotrophic lateral sclerosis (ALS) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats neurodegenerative diseases including Alzheimer disease, Parkinson disease, Huntington's disease, dystonia and/or Amyotrophic lateral sclerosis (ALS) and said primary terpene is selected from the group consisting of terpineol, caryophyllene, myrcene, terpineol, linalool, humulene, eucalyptol, pinene, pulegone, cymene, ocimene, limonene, nerolidol, sabinene, carene, citronellol, geraniol, menthol, borneol, terpinene, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, pinene, terpineol and combinations thereof. According to an embodiment, the primary terpene is selected from the group consisting of caryophyllene, linalool, pinene, terpineol, myrcene, terpineol, limonene, nerolidol, sabinene and combinations thereof. According to various embodiments said primary terpene comprises terpineol, caryophyllene, myrcene, terpineol, linalool, humulene, eucalyptol, pinene, pulegone, cymene, ocimene, limonene, nerolidol, sabinene, carene, citronellol, geraniol, menthol, borneol, terpinene, bisabolol and/or fenchol. According to an embodiment said therapeutic effect treats Alzheimer disease and said the primary terpene is selected from the group consisting of pinene, eucalyptol, linalool, terpineol, myrcene, caryophyllene and combinations thereof. According to an embodiment said therapeutic effect treats Alzheimer disease and said primary terpene is selected from the group consisting of pinene, eucalyptol, linalool and combinations thereof. According to an embodiment said therapeutic effect treats Parkinson disease, Huntington's disease, dystonia and/or Amyotrophic lateral sclerosis (ALS) and said primary terpene is selected from the group consisting caryophyllene, myrcene, linalool, pinene, limonene, nerolidol, sabinene, terpineol and combinations thereof. According to an embodiment said therapeutic effect treats Parkinson disease, Huntington's disease, dystonia and/or Amyotrophic lateral sclerosis (ALS) and said primary terpene is selected from the group consisting myrcene, linalool, pinene, nerolidol and combinations thereof. According to an embodiment, said therapeutic effect treats neurodegenerative diseases including Alzheimer, Parkinson disease, Huntington's disease, dystonia and/or Amyotrophic lateral sclerosis (ALS) and the product comprises a compound selected from the group consisting of linalyl acetate, geranyl acetate, menthol, piperine, eugenol, citronellyl formate, jasmone, benzy acetate, benzyl alcohol and combination thereof. According to an embodiment, the product includes pinene, myrcene and/or terpineol, and pinene, myrcene and/or terpineol forms less than 5% by weight of the total terpene content.

According to an embodiment said therapeutic effect treats multiple sclerosis and said administered product comprises at least one of CBD, THC, CBC, CBG, CBN, CBL, THCV and CBDV. According to an embodiment said therapeutic effect treats multiple sclerosis and said product comprises CBD and optionally THC at CBD to THC weight/weight ratio greater than 1, greater than 2, greater than 3, greater than 4 or greater than 5. According to an embodiment said therapeutic effect treats multiple sclerosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats multiple sclerosis and said primary terpene is selected from the group consisting of caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene, geraniol and combinations thereof. According to an embodiment, the product comprises at least 2 of these terpenes, at least 3, at least 4, or at least 5 of the terpenes. According to an embodiment, the primary terpene is selected from the group consisting of caryophyllene, myrcene, linalool and combinations thereof. According to an embodiment, the primary terpene is selected from the group consisting of caryophyllene, myrcene, linalool, pinene, limonene, eucalyptol and combinations thereof. According to various embodiments said primary terpene comprises caryophyllene, pinene, myrcene, limonene, humulene, citronellol, eucalyptol, caryophyllene oxide, linalool, amyrin, cycloartenol, cymene, terpinene, borneol, pulegol, nerolidol, terpineol, sabinene and/or geraniol.

According to an embodiment said therapeutic effect treats Myasthenia gravis and said administered product comprises at least one of CBD, THC, CBC, CBG, CBN, CBL, THCV and CBDV. According to an embodiment said therapeutic effect treats Myasthenia gravis and said product comprises CBD and optionally THC at CBD to THC weight/weight ratio greater than 1, greater than 2, greater than 3, greater than 4 or greater than 5. According to an embodiment said therapeutic effect treats Myasthenia gravis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Myasthenia gravis and said primary terpene is selected from the group consisting of pinene, terpineol, eucalyptol, pulegone, cymene, ocimene, nerolidol, limonene, linalool, citronellol, geraniol, menthol, caryophyllene, borneol and combinations thereof. According to an embodiment, the product comprises at least 2 of these terpenes, at least 3, at least 4, or at least 5 of the terpenes. According to an embodiment, the primary terpene is selected from the group consisting of terpineol, eucalyptol, linalool and combinations thereof. According to an embodiment, the primary terpene is selected from the group consisting of terpineol, eucalyptol, linalool, pinene, nerolidol, limonene and combinations thereof. According to various embodiments said primary terpene comprises pinene, terpineol, eucalyptol, pulegone, cymene, ocimene, nerolidol, limonene, linalool, citronellol, geraniol, menthol, caryophyllene, and/or borneol. According to an embodiment, the product includes myrcene and/or pinene, and myrcene and/or pinene forms less than 5% by weight of the total terpene content.

According to an embodiment said therapeutic effect treats epilepsy and said administered product comprises at least one of CBD, THC, CBC, CBG, CBN, CBL, THCV and CBDV. According to an embodiment said therapeutic effect treats epilepsy and said product comprises CBD and optionally THC at CBD to THC weight/weight ratio greater than 1, greater than 2, greater than 3, greater than 4 or greater than 5. According to an embodiment said therapeutic effect treats epilepsy and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats epilepsy and said primary terpene is selected from the group consisting of linalool, terpineol, ocimene, myrcene, limonene, caryophyllene and combinations thereof. According to an embodiment, the product comprises at least 2 of these terpenes, at least 3, at least 4, or at least 5 of the terpenes. According to an embodiment, the primary terpene is selected from the group consisting of linalool, terpineol, ocimene and combinations thereof. According to various embodiments said primary terpene comprises linalool, terpineol, ocimene, myrcene, limonene and/or caryophyllene.

According to an embodiment said therapeutic effect treats cancer and/or cancer related symptom and said administered product comprises at least one of CBD, THC, CBC, CBG, CBN, CBL, THCV and CBDV. According to an embodiment said therapeutic effect treats cancer and/or cancer related symptom and said product comprises CBD and optionally THC at CBD to THC weight/weight ratio greater than 1, greater than 2, greater than 3, greater than 4 or greater than 5. According to an embodiment said therapeutic effect treats cancer and/or cancer related symptom and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cancer and/or cancer related symptom and said primary terpene is selected from the group consisting of myrcene, limonene, caryophyllene, caryophyllene oxide, terpinene, citronellol, linalool, geraniol. Menthol. humulene, amyrin, cycloartenol, pinene, camphene, eucalyptol, terpineol, borneol and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, linalool and combinations thereof. According to an embodiment, the primary terpene is selected from the group consisting of limonene, caryophyllene, linalool, citronellol, myrcene, eucalyptol, pinene and combinations thereof. According to various embodiments said primary terpene comprises myrcene, limonene, caryophyllene, caryophyllene oxide, terpinene, citronellol, linalool, geraniol, menthol, humulene, amyrin, cycloartenol, pinene, camphene, eucalyptol, terpineol and/or borneol. According to an embodiment, said therapeutic effect treats cancer and/or cancer related symptom and the product comprises a compound selected from the group consisting of linalyl acetate, geranyl acetate, eugenol, eugenyl acetate, piperine, methylis-oeugenol, Methyleugenol, menthol and combination thereof.

According to an embodiment said therapeutic effect treats stroke and/or traumatic brain injury and said administered product comprises at least one of CBD, THC, CBC, CBG, CBN, CBL, THCV and CBDV. According to an embodiment said therapeutic effect treats stroke and/or traumatic brain injury and said product comprises CBD and optionally THC at CBD to THC weight/weight ratio greater than 1, greater than 2, greater than 3, greater than 4 or greater than 5. According to an embodiment said therapeutic effect treats stroke and/or traumatic brain injury and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats stroke and/or traumatic brain injury and said primary terpene is selected from the group consisting of myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol, carotol and combinations thereof. According to an embodiment, the product comprises at least 2 of these terpenes, at least 3, at least 4, or at least 5 of the terpenes. According to an embodiment, the primary terpene is selected from the group consisting of myrcene, caryophyllene, limonene and combinations thereof. According to an embodiment, the primary terpene is selected from the group consisting of myrcene, caryophyllene, limonene, pinene, humulene, terpineol, thymol, eucalyptol and combinations thereof. According to various embodiments said primary terpene comprises myrcene, terpinene, caryophyllene, caryophyllene oxide, pinene, limonene, humulene, citronellol, eucalyptol, linalool, amyrin, cycloartenol, terpineol, thymol, eucalyptol, geraniol, carvacrol, thymol, cardinol, menth-2-en-1-ol and/or carotol. According to an embodiment, the product includes fenchol and/or borneol, and fenchol and/or borneol forms less than 5% by weight of the total terpene content.

According to an embodiment said therapeutic effect treats addiction, including addiction to drugs, smoking, drinks, food, gambling, sex, and sports and said administered product comprises at least one of CBD, THC, CBC, CBG, CBN, CBL, THCV and CBDV. According to an embodiment said therapeutic effect treats addiction, including addiction to drugs, smoking, drinks, food, gambling, sex, and sports and said product comprises CBD and optionally THC at CBD to THC weight/weight ratio greater than 1, greater than 2, greater than 3, greater than 4 or greater than 5. According to an embodiment said therapeutic effect treats addiction, including addiction to drugs, smoking, drinks, food, gambling, sex, and sports and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats addiction, including addiction to drugs, smoking, drinks, food, gambling, sex, and sports and said primary terpene is selected from the group consisting of caryophyllene, pinene, myrcene, limonene, linalool, terpineol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, terpineol and combinations thereof. According to various embodiments said primary terpene comprises caryophyllene, pinene, myrcene, limonene, linalool and/or terpineol.

According to an embodiment said therapeutic effect treats inflammatory airway diseases including asthma and/or chronic obstructive pulmonary disease (COPD) and said administered product comprises at least one of CBD, THC, CBC, CBG, CBN, CBL, THCV and CBDV. According to an embodiment said therapeutic effect treats inflammatory airway diseases including asthma and/or chronic obstructive pulmonary disease (COPD) and said product comprises CBD and optionally THC at CBD to THC weight/weight ratio greater than 1, greater than 2, greater than 3, greater than 4 or greater than 5. According to an embodiment said therapeutic effect treats inflammatory airway diseases including asthma and/or chronic obstructive pulmonary disease (COPD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammatory airway diseases including asthma and/or chronic obstructive pulmonary disease (COPD) and said primary terpene is selected from the group consisting of pinene, eucalyptol, limonene, humulene, camphene, caryophyllene and combinations thereof. According to an embodiment, the product comprises at least 2 of these terpenes, at least 3, at least 4, or at least 5 of the terpenes. According to an embodiment, the primary terpene is selected from the group consisting of pinene, eucalyptol, humulene, and combinations thereof. According to various embodiments said primary terpene comprises pinene, eucalyptol, limonene, humulene, camphene and/or caryophyllene.

According to an embodiment said therapeutic effect treats cerebral palsy (CP) and said administered product comprises at least one of CBD, THC, CBC, CBG, CBN, CBL, THCV and CBDV. According to an embodiment said therapeutic effect treats cerebral palsy (CP) and said product comprises CBD and optionally THC at CBD to THC weight/weight ratio greater than 1, greater than 2, greater than 3, greater than 4 or greater than 5. According to an embodiment said therapeutic effect treats cerebral palsy (CP) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cerebral palsy (CP) and said primary terpene is selected from the group consisting of myrcene, pinene, linalool, humulene, nerolidol, limonene, terpineol, sabinene and combinations thereof. According to an embodiment, the product comprises at least 2 of these terpenes, at least 3, at least 4, or at least 5 of the terpenes. According to an embodiment, the primary terpene is selected from the group consisting of myrcene, pinene, nerolidol and combinations thereof. According to an embodiment, the primary terpene is selected from the group consisting of linalool, limonene, sabinene and combinations thereof. According to various embodiments said primary terpene comprises myrcene, pinene, linalool, humulene, nerolidol, limonene, terpineol and/or sabinene.

According to an embodiment said therapeutic effect treats autism and/or autism spectrum disorder and said product comprises at least one of CBD, THC, CBC, CBG, CBN, CBL, THCV and CBDV. According to an embodiment said therapeutic effect treats autism and/or autism spectrum disorder and said administered product comprises CBD and optionally THC at CBD to THC weight/weight ratio greater than 1, greater than 2, greater than 3, greater than 4 or greater than 5. According to an embodiment said therapeutic effect treats autism and/or autism spectrum disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats autism and/or autism spectrum disorder and said primary terpene is selected from the group consisting of limonene, caryophyllene, pinene, myrcene, humulene, citronellol, eucalyptol, linalool, terpineol, ocimene, geraniol, menthol, borneol, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of pinene, eucalyptol, linalool and combinations thereof. According to an embodiment, the primary terpene is selected from the group consisting of pinene, eucalyptol, linalool, caryophyllene, myrcene, terpineol and combinations thereof. According to various embodiments said primary terpene comprises limonene, caryophyllene, pinene, myrcene, humulene, citronellol, eucalyptol, linalool, terpinenol, ocimene, geraniol, menthol, borneol, bisabolol and/or fenchol.

According to an embodiment said therapeutic effect treats hyperactivity disorder (ADHD), attention deficit disorder (ADD) and/or obsessive-compulsive disorder (OCD) and said administered product comprises at least one of CBD, THC, CBC, CBG, CBN, CBL, THCV and CBDV. According to an embodiment said therapeutic effect treats hyperactivity disorder (ADHD), attention deficit disorder (ADD) and/or obsessive-compulsive disorder (OCD) and said product comprises CBD and optionally THC at CBD to THC weight/weight ratio greater than 1, greater than 2, greater than 3, greater than 4 or greater than 5. According to an embodiment said therapeutic effect treats hyperactivity disorder (ADHD), attention deficit disorder (ADD) and/or obsessive-compulsive disorder (OCD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hyperactivity disorder (ADHD), attention deficit disorder (ADD) and/or obsessive-compulsive disorder (OCD) and said primary terpene is selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof. According to an embodiment, the product comprises at least 2 of these terpenes, at least 3, at least 4, or at least 5 of the terpenes. According to an embodiment said therapeutic effect treats hyperactivity disorder (ADHD) and/or attention deficit disorder (ADD) and said primary terpene is selected from the group consisting of linalool, eucalyptol, myrcene, and combinations thereof. According to an embodiment, the primary terpene is selected from the group consisting of linalool, eucalyptol, myrcene, caryophyllene, pinene, borneol and combinations thereof. According to an embodiment said therapeutic effect treats obsessive-compulsive disorder (OCD) and said primary terpene is selected from the group consisting of limonene, eucalyptol, pinene and combination thereof. According to an embodiment, the primary terpene is selected from the group consisting of linalool, caryophyllene, borneol and combination thereof. According to various embodiments said primary terpene comprises linalool, eucalyptol, terpineol, myrcene, pinene, pulegone, caryophyllene, geraniol, citronellol, menthol, caryophyllene, pinene, borneol, bisabolol and/or fenchol.

According to an embodiment said therapeutic effect treats Tourette syndrome, motor spasms, motor tics and/or vocal tics and said administered product comprises at least one of CBD, THC, CBC, CBG, CBN, CBL, THCV and CBDV. According to an embodiment said therapeutic effect treats Tourette syndrome, motor spasms, motor tics and/or vocal tics and said product comprises CBD and optionally THC at CBD to THC weight/weight ratio greater than 1, greater than 2, greater than 3, greater than 4 or greater than 5. According to an embodiment said therapeutic effect treats Tourette syndrome, motor spasms, motor tics and/or vocal tics and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Tourette syndrome, motor spasms, motor tics and/or vocal tics and said primary terpene is selected from the group consisting of myrcene, linalool, limonene, pinene, nerolidol, caryophyllene, eucalyptol, geraniol, menthol, borneol, bisabolol, fenchol and combinations thereof. According to an embodiment, the product comprises at least 2 of these terpenes, at least 3, at least 4, or at least 5 of the terpenes. According to an embodiment, the primary terpene is selected from the group consisting of myrcene, limonene, nerolidol and combinations thereof. According to an embodiment, the primary terpene is selected from the group consisting of linalool, pinene, eucalyptol and combinations thereof. According to various embodiments said primary terpene comprises myrcene, linalool, limonene, pinene, nerolidol, caryophyllene, eucalyptol, geraniol, menthol, borneol, bisabolol and/or fenchol.

According to an embodiment said therapeutic effect treats psoriasis and said administered product comprises at least one of CBD, THC, CBC, CBG, CBN, CBL, THCV and CBDV. According to an embodiment said therapeutic effect treats psoriasis and said product comprises CBD and optionally THC at CBD to THC weight/weight ratio greater than 1, greater than 2, greater than 3, greater than 4 or greater than 5. According to an embodiment said therapeutic effect treats psoriasis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats psoriasis and said primary terpene is selected from the group consisting of linalool, caryophyllene, limonene and combinations thereof. According to an embodiment, the product comprises at least 2 of these terpenes or all 3 of the terpenes. According to an embodiment, the primary terpene is selected from the group consisting of linalool, caryophyllene, limonene and combinations thereof. According to various embodiments said primary terpene comprises linalool, caryophyllene and/or limonene.

According to an embodiment said therapeutic effect treats dermatophytes and said administered product comprises at least one of CBD, THC, CBC, CBG, CBN, CBL, THCV and CBDV. According to an embodiment said therapeutic effect treats dermatophytes and said product comprises CBD and optionally THC at CBD to THC weight/weight ratio greater than 1, greater than 2, greater than 3, greater than 4 or greater than 5. According to an embodiment said therapeutic effect treats dermatophytes and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats dermatophytes and said primary terpene is selected from the group consisting of limonene, caryophyllene, caryophyllene oxide, nerolidol, eucalyptol, terpinene, cycloartenol, amyrin, campene and combinations thereof. According to an embodiment, the product comprises at least 2 of these terpenes, at least 3, at least 4, or at least 5 of the terpenes. According to an embodiment, the primary terpene is selected from the group consisting of limonene, caryophyllene, caryophyllene oxide and combinations thereof. According to an embodiment, the primary terpene is selected from the group consisting of limonene, caryophyllene, caryophyllene oxide, nerolidol, eucalyptol, terpinene and combinations thereof. According to various embodiments said primary terpene comprises limonene, caryophyllene, caryophyllene oxide, nerolidol, eucalyptol, terpinene, cycloartenol, amyrin and/or campene.

According to an embodiment said therapeutic effect treats Candida infection and said administered product comprises at least one of CBD, THC, CBC, CBG, CBN, CBL, THCV and CBDV. According to an embodiment said therapeutic effect treats Candida infection and said product comprises CBD and optionally THC at CBD to THC weight/weight ratio greater than 1, greater than 2, greater than 3, greater than 4 or greater than 5. According to an embodiment said therapeutic effect treats Candida infection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Candida infection and said primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ionone and combinations thereof. According to an embodiment, the product comprises at least 2 of these terpenes, at least 3, at least 4, or at least 5 of the terpenes. According to an embodiment, the primary terpene is selected from the group consisting of thymol, carvacrol, eugenol and combinations thereof. According to an embodiment, the primary terpene is selected from the group consisting of thymol, carvacrol, eugenol, nerolidol, linalool, farnesol, citronellol, geraniol, citral and combinations thereof. According to various embodiments said primary terpene comprises pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral and/or ionone.

According to an embodiment said therapeutic effect treats leishmaniasis and said administered product comprises at least one of CBD, THC, CBC, CBG, CBN, CBL, THCV and CBDV. According to an embodiment said therapeutic effect treats leishmaniasis and said product comprises CBD and optionally THC at CBD to THC weight/weight ratio greater than 1, greater than 2, greater than 3, greater than 4 or greater than 5. According to an embodiment said therapeutic effect treats leishmaniasis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats leishmaniasis and said primary terpene is selected from the group consisting of linalool, nerolidol, pinene, caryophyllene and/or eucalyptol. According to an embodiment said primary terpene is linalool and/or nerolidol.

According to an embodiment said therapeutic effect treats Methycillin-resistant Staphylococcus aureus (MRSA) and said administered product comprises at least one of CBD, THC, CBC, CBG, CBN, CBL, THCV and CBDV. According to an embodiment said therapeutic effect treats Methycillin-resistant Staphylococcus aureus (MRSA) and said product comprises CBD and optionally THC at CBD to THC weight/weight ratio greater than 1, greater than 2, greater than 3, greater than 4 or greater than 5. According to an embodiment said therapeutic effect treats Methycillin-resistant Staphylococcus aureus (MRSA) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Methycillin-resistant Staphylococcus aureus (MRSA) and said primary terpene is selected from the group consisting of pinene, caryophyllene, caryophyllene oxide, limonene, nerolidol, cycloartenol, amyrin and combinations thereof. According to an embodiment, the product comprises at least 2 of these terpenes, at least 3, at least 4, or at least 5 of the terpenes. According to an embodiment, the primary terpene is selected from the group consisting of pinene, caryophyllene, limonene, amyrin and combinations thereof. According to various embodiments said primary terpene comprises pinene, caryophyllene, caryophyllene oxide, limonene, nerolidol, cycloartenol and/or amyrin.

According to an embodiment said therapeutic effect treats malaria and said administered product comprises at least one of CBD, THC, CBC, CBG, CBN, CBL, THCV and CBDV. According to an embodiment said therapeutic effect treats malaria and said product comprises CBD and optionally THC at CBD to THC weight/weight ratio greater than 1, greater than 2, greater than 3, greater than 4 or greater than 5. According to an embodiment said therapeutic effect treats malaria and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats malaria and said primary terpene is selected from the group consisting of caryophyllene, nerolidol, pinene, eucalyptol, limonene and combinations thereof. According to an embodiment, the product comprises at least 2 of these terpenes, at least 3, at least 4, or at least 5 of the terpenes. According to an embodiment, the primary terpene is selected from the group consisting of caryophyllene, nerolidol, pinene and combinations thereof. According to various embodiments said primary terpene comprises caryophyllene, nerolidol, pinene, eucalyptol and/or limonene.

According to an embodiment said therapeutic effect treats acne and said administered product comprises at least one of CBD, THC, CBC, CBG, CBN, CBL, THCV and CBDV. According to an embodiment said therapeutic effect treats acne and said product comprises CBD and optionally THC at CBD to THC weight/weight ratio greater than 1, greater than 2, greater than 3, greater than 4 or greater than 5. According to an embodiment said therapeutic effect treats acne) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats acne and said primary terpene is selected from the group consisting of limonene, linalool, nerolidol, pinene, terpineol and combinations thereof. According to an embodiment, the product comprises at least 2 of these terpenes, at least 3, at least 4, or all 5 of the terpenes. According to an embodiment, the primary terpene is selected from the group consisting of limonene, pinene, terpineol and combinations thereof. According to various embodiments said primary terpene comprises limonene, linalool, nerolidol, pinene and/or terpineol.

According to an embodiment said therapeutic effect treats allergy and said administered product comprises at least one of CBD, THC, CBC, CBG, CBN, CBL, THCV and CBDV. According to an embodiment said therapeutic effect treats allergy and said product comprises CBD and optionally THC at CBD to THC weight/weight ratio greater than 1, greater than 2, greater than 3, greater than 4 or greater than 5. According to an embodiment said therapeutic effect treats allergy and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats allergy and said primary terpene is selected from the group consisting of camphene, pinene, borneol, caryophyllene, geraniol and combinations thereof. According to an embodiment, the product comprises at least 2 of these terpenes, at least 3, at least 4, or all 5 of the terpenes. According to an embodiment, the primary terpene is selected from the group consisting of camphene, pinene, caryophyllene and combinations thereof. According to various embodiments said primary terpene comprises camphene, pinene, borneol, caryophyllene and/or geraniol.

According to an embodiment said therapeutic effect treats osteoporosis and said administered product comprises at least one of CBD, THC, CBC, CBG, CBN, CBL, THCV and CBDV. According to an embodiment said therapeutic effect treats osteoporosis and said product comprises CBD and optionally THC at CBD to THC weight/weight ratio greater than 1, greater than 2, greater than 3, greater than 4 or greater than 5. According to an embodiment said therapeutic effect treats osteoporosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats osteoporosis and said the primary terpene is selected from the group consisting of caryophyllene, caryophyllene oxide, carene, eucalyptol, sabinene, pinene, limonene, borneol, thymol, camphor and combinations thereof. According to an embodiment, the product comprises at least 2 of these terpenes, at least 3, at least 4, or at least 5 of the terpenes. According to an embodiment, the primary terpene is selected from the group consisting of caryophyllene, carene, camphor and combinations thereof. According to an embodiment, the primary terpene is selected from the group consisting of caryophyllene, carene, camphor, pinene, limonene, borneol and combinations thereof. According to various embodiments said primary terpene comprises caryophyllene oxide, eucalyptol, sabinene, pinene, limonene, thymol and/or camphor. According to an embodiment, said therapeutic effect treats osteoporosis said product and comprises a herbal extract selected from the group consisting of extracts of Angelica sinensis, Cimicifuga (Actaea) racemosa, Dioscorea villosa, Trifolium pratense and combinations thereof.

According to an embodiment said therapeutic effect treats sleep disorder and/or insomnia and said administered product comprises at least one of CBD, THC, CBC, CBG, CBN, CBL, THCV and CBDV. According to an embodiment said therapeutic effect treats sleep disorder and/or insomnia and said product comprises CBD and optionally THC at CBD to THC weight/weight ratio greater than 1, greater than 2, greater than 3, greater than 4 or greater than 5. According to an embodiment said therapeutic effect treats sleep disorder and/or insomnia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats sleep disorder and/or insomnia and said the primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol, citral and combinations thereof. According to an embodiment, the product comprises at least 2 of these terpenes, at least 3, at least 4, or at least 5 of the terpenes. According to an embodiment, the primary terpene is selected from the group consisting of linalool, terpineol, myrcene and combinations thereof. According to an embodiment, the primary terpene is selected from the group consisting of linalool, terpineol, myrcene, caryophyllene, terpinene, borneol and combinations thereof. According to an embodiment, the primary terpene comprises terpineol, citronellol and/or linalool. According to various embodiments said primary terpene comprises terpineol, terpinene, citronellol, eucalyptol, nerolidol, sabinene, pinene, limonene, borneol, fenchol, bisabolol, phytol and/or humulene. According to an embodiment, said therapeutic effect treats sleep disorder and/or insomnia and the product comprises a compound selected from the group consisting of linalyl acetate and/or chamazulene. According to an embodiment, said therapeutic effect treats sleep disorder and/or insomnia and said product comprises a herbal extract selected from the group consisting of extracts of Avena sativa, Cimicifuga (Actaea) racemosa, Humulus lupulus, Hypericum perforatum, Lavendula officinalis, Melissa officinalis, Passiflora incarnata, Valeriana officinalis and combinations thereof According to an embodiment, the product includes caryophyllene, and caryophyllene forms less than 5% by weight of the total terpene content.

According to an embodiment said therapeutic effect treats anxiety, irritability, nervousness, restless, stress, depression, general discomfort, mood problems and/or affect disorder and said administered product comprises at least one of CBD, THC, CBC, CBG, CBN, CBL, THCV and CBDV. According to an embodiment said therapeutic effect treats anxiety, irritability, nervousness, restless, stress, depression, general discomfort, mood problems and/or affect disorder and said product comprises CBD and optionally THC at CBD to THC weight/weight ratio greater than 1, greater than 2, greater than 3, greater than 4 or greater than 5. According to an embodiment said therapeutic effect treats anxiety, irritability, nervousness, restless, stress, depression, general discomfort, mood problems and/or affect disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats anxiety, irritability, nervousness, restless, stress, depression, general discomfort, mood problems and/or affect disorder and said the primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol, phellandrene and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, terpineol, caryophyllene, pinene, myrcene and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol phellandrene and/or fenchol. According to an embodiment, said therapeutic effect treats anxiety, irritability, nervousness, restless, stress, depression, general discomfort, mood problems and/or affect disorder and the product comprises a compound selected from the group consisting of linalyl acetate, geranyl acetate, menthol, piperine, eugenol, citronellyl formate, jasmone, benzy acetate, benzyl alcohol and combinations thereof. According to an embodiment, said therapeutic effect treats anxiety, irritability, nervousness, restless, stress, depression, general discomfort, mood problems and/or affect disorder and said product comprises a herbal extract selected from the group consisting of extracts of Avena sativa, Bacopa monnieri, Centella (Hydrocotyl) asiatica, Humulus lupulus, Hypericum perforatum, Melissa officinalis, Passiflora incarnata, Valeriana officinalis and combinations thereof. According to an embodiment, the product includes bisabolol, nerolidol, terpinene, myrcene and/or pinene bisabolol, nerolidol, terpinene, myrcene and/or pinene, and bisabolol, nerolidol, terpinene and/or pinene forms less than 5% by weight of the total terpene content.

According to an embodiment said therapeutic effect treats difficulty in concentration, memory loss, and/or dementia and said administered product comprises at least one of CBD, THC, CBC, CBG, CBN, CBL, THCV and CBDV. According to an embodiment said therapeutic effect treats difficulty in concentration, memory loss, and/or dementia and said product comprises CBD and optionally THC at CBD to THC weight/weight ratio greater than 1, greater than 2, greater than 3, greater than 4 or greater than 5. According to an embodiment said therapeutic effect treats difficulty in concentration, memory loss, and/or dementia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats difficulty in concentration, memory loss, and/or dementia and said the primary terpene is selected from the group consisting of pinene, pulegone, eucalyptol, terpineol, cymene, ocimene, limonene, linalool, caryophyllene, humulene, careen, bisabolol, guaiol, terpinolene and combinations thereof. According to an embodiment, the product comprises at least 2 of these terpenes, at least 3, at least 4, or at least 5 of the terpenes. According to an embodiment, the primary terpene is selected from the group consisting of eucalyptol, pinene, limonene and combinations thereof. According to an embodiment, the primary terpene is selected from the group consisting of eucalyptol, pinene, limonene, terpineol, humulene, carene and combinations thereof. According to various embodiments said primary terpene comprises pinene, pulegone, eucalyptol, terpineol, cymene, ocimene, limonene, linalool, caryophyllene, humulene and/or carene. According to an embodiment said therapeutic effect treats difficulty in concentration, memory loss, and/or dementia and said product comprises a herbal extract selected from the group consisting of extracts of Asphalentum ponjabianum-mumio, Bacopa monnieri, Centella (hydrocotyl) asiatica, Eleutherococcus senticosus, Ginkgo biloba, Lepidium meyenii, Lycium barbarum, Paullinia cupana, Rhodiola rosea and combinations thereof.

According to an embodiment said therapeutic effect treats muscle pain, myalgia, spasticity, muscle tension, cramps and/or spasms and said administered product comprises at least one of CBD, THC, CBC, CBG, CBN, CBL, THCV and CBDV. According to an embodiment said therapeutic effect treats muscle pain, myalgia, spasticity, muscle tension, cramps and/or spasms and said product comprises CBD and optionally THC at CBD to THC weight/weight ratio greater than 1, greater than 2, greater than 3, greater than 4 or greater than 5. According to an embodiment said therapeutic effect treats muscle pain, myalgia, spasticity, muscle tension, cramps and/or spasms and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats muscle pain, myalgia, spasticity, muscle tension, cramps and/or spasms and said primary terpene is selected from the group consisting of myrcene, pinene, nerolidol, linalool, humulene, caryophyllene, limonene, bisabolol, sabinene, citral, eucalyptol, terpineol, geraniol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, limonene and combinations thereof. According to an embodiment, the primary terpene is selected from the group consisting of myrcene, pinene, limonene, nerolidol, linalool, caryophyllene, sabinene and combinations thereof. According to an embodiment, the primary terpene is selected from the group consisting of myrcene, pinene, nerolidol, linalool, humulene and combinations thereof. According to various embodiments said primary terpene comprises myrcene, pinene, nerolidol, linalool, humulene, caryophyllene, limonene, bisabolol, sabinene, citral, eucalyptol, geraniol and/or terpineol. According to an embodiment said therapeutic effect treats cramps and/or spasms and said primary terpene is selected from the group consisting of myrcene, pinene, nerolidol, sabinene, eucalyptol and combinations thereof. According to an embodiment said primary terpene is selected from the group consisting of myrcene, linalool, eucalyptol and combinations thereof. According to an embodiment said therapeutic effect treats muscle pain, myalgia, spasticity, muscle tension, cramps and/or spasms and said product comprises a herbal extract selected from the group consisting of extracts of Angelica sinensis, Piscidia erythrina, Tanacetum parthenium, Viburnum opulus, Boswellia spp, Capsicum spp, Corydalis yanhusuo, Harpagophytum procumbens, Rosmarinus officinalis, Salix alba, Zingiber officinalis, chamaemelum, Valeriana edulis and combinations thereof.

According to an embodiment said therapeutic effect treats fatigue, weakness, decreased mental energy, decreased physical energy, dizziness and/or deficits in balance and said administered product comprises at least one of CBD, THC, CBC, CBG, CBN, CBL, THCV and CBDV. According to an embodiment said therapeutic effect treats fatigue, weakness, decreased mental energy, decreased physical energy, dizziness and/or deficits in balance and said product comprises CBD and optionally THC at CBD to THC weight/weight ratio greater than 1, greater than 2, greater than 3, greater than 4 or greater than 5. According to an embodiment said therapeutic effect treats fatigue, weakness, decreased mental energy, decreased physical energy, dizziness and/or deficits in balance and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fatigue, weakness, decreased mental energy, decreased physical energy, dizziness and/or deficits in balance and said the primary terpene is selected from the group consisting of pinene, limonene, terpinene, eucalyptol, myrcene, terpineol, linalool, caryophyllene, borneol and combinations thereof. According to an embodiment, the product comprises at least 2 of these terpenes, at least 3, at least 4, or at least 5 of the terpenes. According to an embodiment, the primary terpene is selected from the group consisting of pinene, terpinene, eucalyptol and combinations thereof. According to an embodiment, the primary terpene is selected from the group consisting of limonene, myrcene, borneol and combinations thereof. According to various embodiments said primary terpene comprises terpinene, myrcene, borneol and/or caryophyllene. According to an embodiment said therapeutic effect treats fatigue, weakness, decreased mental energy, decreased physical energy, dizziness and/or deficits in balance and said product comprises a herbal extract selected from the group consisting of extracts of Eleutherococcus senticosus, Ganoderma lucidum, Grifola frondosa, Lentinula edodes, Lepidium meyenii, Panax ginseng, Rhodiola rosea and combinations thereof.

According to an embodiment said therapeutic effect treats itchy skin, pruritus and/or chronic pruritus, wrinkles, stretch marks and/or skin burns and said administered product comprises at least one of CBD, THC, CBC, CBG, CBN, CBL, THCV and CBDV. According to an embodiment said therapeutic effect treats itchy skin, pruritus and/or chronic pruritus, wrinkles, stretch marks and/or skin burns and said product comprises CBD and optionally THC at CBD to THC weight/weight ratio greater than 1, greater than 2, greater than 3, greater than 4 or greater than 5. According to an embodiment said therapeutic effect treats itchy skin, pruritus and/or chronic pruritus, wrinkles, stretch marks and/or skin burns and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats itchy skin, pruritus and/or chronic pruritus, wrinkles, stretch marks and/or skin burns and said the primary terpene is selected from the group consisting of linalool limonene, nerolidol, pinene terpineol, myrcene, geraniol, nerol, citronellol, farnesol, carotol, sabinene and combinations thereof. According to an embodiment, the product comprises at least 2 of these terpenes, at least 3, at least 4, or at least 5 of the terpenes. According to an embodiment, the primary terpene is selected from the group consisting of linalool, terpineol, geraniol and combinations thereof. According to an embodiment said therapeutic effect treats skin burns and said the primary terpene is linalool and/or sabinene. According to an embodiment said therapeutic effect treats wrinkles, stretch marks and said the primary terpene is selected from the group consisting of geraniol, linalool, citronellol and combinations thereof. According to various embodiments said primary terpene comprises limonene, nerolidol, pinene myrcene, nerol, citronellol, farnesol, linalool, terpineol, geraniol and/or carotol. According to an embodiment, said therapeutic effect treats itchy skin, pruritus and/or chronic pruritus, wrinkles, stretch marks and/or skin burns and the product comprises a compound selected from the group consisting of linalyl acetate, citronellyl formate, eugenol, benzy acetate, benzyl alcohol, and combinations thereof. According to an embodiment said therapeutic effect treats itchy skin, pruritus and/or chronic pruritus, wrinkles, stretch marks and/or skin burns and said product comprises a herbal extract selected from the group consisting of extracts of Glycyrrhiza glabra, Plantago spp, Symphytum officinalis, Trifolium pratense, Trigonella foenum graecum, Ulmus rubra\fulva, Verbascum thapsus and combinations thereof.

According to an embodiment said therapeutic effect treats headache and/or migraine and said administered product comprises at least one of CBD, THC, CBC, CBG, CBN, CBL, THCV and CBDV. According to an embodiment said therapeutic effect treats headache and/or migraine and said product comprises CBD and optionally THC at CBD to THC weight/weight ratio greater than 1, greater than 2, greater than 3, greater than 4 or greater than 5. According to an embodiment said therapeutic effect treats headache and/or migraine and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats headache and/or migraine and said the primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to an embodiment, the primary terpene is selected from the group consisting of myrcene, linalool, caryophyllene, limonene, nerolidol, fenchol and combinations thereof. According to various embodiments said primary terpene comprises of myrcene, linalool, caryophyllene, eucalyptol, limonene, nerolidol, fenchol, humulene, guaiol, borneol, pinene, terpinene, terpineol and/or bisabolol. According to an embodiment, the product includes linalool, bisabolol and/or humulene wherein linalool, bisabolol and/or humulene forms less than 5% by weight of the total terpene content. According to an embodiment said therapeutic effect treats headache and/or migraine and said product comprises a herbal extract selected from the group consisting of extracts of Angelica sinensis, Corydalis yanhusuo, Piscidia erythrina, Rosmarinus officinalis, Stachys betonica, Tanacetum parthenium, verbena officinalis and combinations thereof.

According to an embodiment said therapeutic effect treats weight gain and/or reduces appetite and said administered product comprises at least one of CBD, THC, CBC, CBG, CBN, CBL, THCV and CBDV. According to an embodiment said therapeutic effect treats weight gain and/or reduces appetite and said product comprises CBD and optionally THC at CBD to THC weight/weight ratio greater than 1, greater than 2, greater than 3, greater than 4 or greater than 5. According to an embodiment said therapeutic effect treats weight gain and/or reduces appetite and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats weight gain and/or reduces appetite and said the primary terpene is selected from the group consisting of eucalyptol, caryophyllene, limonene, humulene, phytol and combinations thereof. According to an embodiment, the product comprises at least 2 of these terpenes, at least 3, at least 4, or at least 5 of the terpenes. According to an embodiment, the primary terpene is selected from the group consisting of eucalyptol, limonene, humulene and combinations thereof. According to various embodiments [00256] said primary terpene comprises caryophyllene, eucalyptol, limonene, humulene and/or phytol. According to an embodiment said therapeutic effect treats weight gain and/or reduces appetite and said product comprises a herbal extract selected from the group consisting of extracts of Ephedra sinica, Foeniculum vulgare, Fucus vesiculosus, Garcinia cambogia, Gymnema sylvestre, laminaria spp, Paullinia cupana and combinations thereof.

According to an embodiment said therapeutic effect treats digestive problems, intestinal disorders, gastrointestinal pain, abdominal pain (colic), pelvic pain, constipation and/or diarrhea and said administered product comprises at least one of CBD, THC, CBC, CBG, CBN, CBL, THCV and CBDV. According to an embodiment said therapeutic effect treats digestive problem, intestinal disorder, gastrointestinal pain, abdominal pain (colic), pelvic pain, constipation and/or diarrhea and said product comprises CBD and optionally THC at CBD to THC weight/weight ratio greater than 1, greater than 2, greater than 3, greater than 4 or greater than 5. According to an embodiment said therapeutic effect treats digestive problem, intestinal disorder, gastrointestinal pain, abdominal pain (colic), pelvic pain, constipation and/or diarrhea and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats digestive problem, intestinal disorder, gastrointestinal pain, abdominal pain (colic), pelvic pain, constipation and/or diarrhea and said the primary terpene is selected from the group consisting of limonene, cymene, ocimene, terpinene, caryophyllene, citronellol, sabinene, linalool, pinene, myrcene, germacrene, geraniol, nerolidol, menthol, humulene, fenchol, borneol and combinations thereof. According to an embodiment, the product comprises at least 2 of these terpenes, at least 3, at least 4, or at least 5 of the terpenes. According to an embodiment, the primary terpene is selected from the group consisting of limonene, sabinene, caryophyllene and combinations thereof. According to an embodiment, the primary terpene is selected from the group consisting of limonene, sabinene, caryophyllene, citronellol, linalool, myrcene, nerolidol and combinations thereof According to various embodiments said primary terpene comprises limonene, cymene, ocimene, terpinene, caryophyllene, citronellol, sabinene, linalool, pinene, myrcene, germacrene, geraniol, nerolidol, menthol, humulene, fenchol and/or borneol. According to an embodiment, said therapeutic effect treats digestive problem, intestinal disorder, gastrointestinal pain, abdominal pain (colic), pelvic pain, constipation and/or diarrhea and the product further comprises piperine. According to an embodiment said therapeutic effect treats digestive problems, intestinal disorders, gastrointestinal pain, abdominal pain (colic), pelvic pain, constipation and/or diarrhea and said product comprises a herbal extract selected from the group consisting of extracts of Angelica sinensis, Piscidia erythrina, Tanacetum parthenium, Viburnum opulus, Boswellia spp, Capsicum spp, Corydalis yanhusuo, Harpagophytum procumbens, Rosmarinus officinalis, Salix alba, Zingiber officinalis and combinations thereof. According to an embodiment, the product includes pinene, bisabolol and/or limonene, and pinene, bisabolol and/or limonene forms less than 5% by weight of the total terpene content.

According to an embodiment said therapeutic effect treats lack of appetite, nausea, vomiting and/or anorexia and said administered product comprises at least one of CBD, THC, CBC, CBG, CBN, CBL, THCV and CBDV. According to an embodiment said therapeutic effect treats lack of appetite, nausea, vomiting and/or anorexia and said product comprises CBD and optionally THC at CBD to THC weight/weight ratio greater than 1, greater than 2, greater than 3, greater than 4 or greater than 5. According to an embodiment said therapeutic effect treats lack of appetite, nausea, vomiting and/or anorexia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats lack of appetite, nausea, vomiting and/or anorexia and said the primary terpene is selected from the group consisting of linalool, citronellol, geraniol, menthol, humulene, bisabolol, borneol, fenchol, linalool, limonene, terpineol, pinene, caryophyllene, myrcene, cymene, terpinene, valencene and combinations thereof. According to an embodiment, the product comprises at least 2 of these terpenes, at least 3, at least 4, or at least 5 of the terpenes. According to an embodiment, the primary terpene is selected from the group consisting of citronellol, linalool caryophyllene and combinations thereof. According to an embodiment, the primary terpene is selected from the group consisting of citronellol, linalool caryophyllene, limonene, myrcene, nerolidol and combinations thereof According to various embodiments said primary terpene comprises linalool, citronellol, geraniol, menthol, humulene, bisabolol, borneol, fenchol, linalool, limonene, terpineol, pinene, caryophyllene, myrcene, cymene and/or terpinene. According to an embodiment, the product includes pinene. borneol, fenchol, terpinene and/or terpineol, and pinene, bisabolol and/or limonene forms less than 5% by weight of the total terpene content.

According to an embodiment said therapeutic effect treats joint pain, joint stiffness, arthralgia, arthritis and/or osteoarthritis and said administered product comprises at least one of CBD, THC, CBC, CBG, CBN, CBL, THCV and CBDV. According to an embodiment said therapeutic effect treats joint pain, joint stiffness, arthralgia, arthritis and/or osteoarthritis and said product comprises CBD and optionally THC at CBD to THC weight/weight ratio greater than 1, greater than 2, greater than 3, greater than 4 or greater than 5. According to an embodiment said therapeutic effect treats joint pain, joint stiffness, arthralgia, arthritis and/or osteoarthritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats a joint pain, joint stiffness, arthralgia, arthritis and/or osteoarthritis and said the primary terpene is selected from the group consisting of myrcene, linalool, caryophyllene, eucalyptol, pinene, nerolidol, sabinene, limonene, terpinene, menth-2-en-1-ol, terpineol, geraniol, guaiol, humulene, bisabolol, borneol, fenchol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, sabinene and combinations thereof. According to an embodiment, the primary terpene is selected from the group consisting of myrcene, pinene, sabinene linalool, caryophyllene, limonene and combinations thereof. According to various embodiments said primary terpene comprises myrcene, linalool, caryophyllene, eucalyptol, pinene, nerolidol, sabinene, limonene, terpinene, menth-2-en-1-ol, geraniol, guaiol, humulene, bisabolol, borneol, fenchol 1 and/or terpineol. According to an embodiment, said therapeutic effect treats joint pain, joint stiffness, arthralgia, arthritis and/or osteoarthritis and the product comprises a compound selected from the group consisting of linalyl acetate, geranyl acetate, eugenol, methylis-oeugenol, menthol, eugenyl acetate, piperine and combinations thereof. According to an embodiment said therapeutic effect treats joint pain, joint stiffness, arthralgia, arthritis and/or osteoarthritis and said product comprises a herbal extract selected from the group consisting of extracts of Boswellia spp, Capsicum spp, Corydalis yanhusuo, Harpagophytum procumbens, Piscidia erythrina, Salix alba, Zingiber officinalis and combinations thereof. According to an embodiment, the product optionally includes humulene, pinene, bisabolol, borneol, fenchol, guaiol, linalool, ocimene, terpineol, terpinene and/or caryophyllene, and humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, night sweat, palpitation, irritability and/or restlessness and said administered product comprises at least one of CBD, THC, CBC, CBG, CBN, CBL, THCV and CBDV. According to an embodiment said therapeutic effect treats hot flashes, night sweat, palpitation, irritability and/or restlessness and said product comprises CBD and optionally THC at CBD to THC weight/weight ratio greater than 1, greater than 2, greater than 3, greater than 4 or greater than 5. According to an embodiment said therapeutic effect treats hot flashes, night sweat, palpitation, irritability and/or restlessness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hot flashes, night sweat, palpitation, irritability and/or restlessness and said the primary terpene is selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof. According to an embodiment, the product comprises at least 2 of these terpenes, at least 3, at least 4, or at least 5 of the terpenes. According to an embodiment, the primary terpene is selected from the group consisting of linalool, limonene, eucalyptol and combinations thereof. According to an embodiment, the primary terpene is selected from the group consisting of linalool, limonene, terpineol, geraniol, citronellol, eucalyptol and combinations thereof. According to various embodiments said primary terpene comprises caryophyllene, myrcene, humulene, pinene, linalool, limonene, nerolidol, terpinene, terpineol, geraniol, carene, citronellol, eucalyptol, sabinene, myrtenol, bisabolol, borneol and/or fenchol. According to an embodiment, said therapeutic effect treats hot flashes, night sweat, palpitation, irritability and/or restlessness and the product further comprises a compound selected from the group consisting of linalyl acetate, geranyl acetate, citronellyl formate and combinations thereof. According to an embodiment said therapeutic effect treats hot flashes, night sweat, palpitation, irritability and/or restlessness and said product comprises a herbal extract selected from the group consisting of extracts of Angelica sinensis, Anemarrhenae asphodeloides, Cimicifuga (Actaea) racemosa, Dioscorea villosa, Salvia officinalis, Salvia officinalis, Rehmania glutinosa preparata and combinations thereof. According to an embodiment, the product optionally includes pinene, bisabolol and/or terpineol, and pinene, bisabolol and/or terpineol forms less than 5% by weight of the total terpene content.

According to an embodiment said therapeutic effect treats inflammation, oxidative stress and/or paresthesia and said administered product comprises at least one of CBD, THC, CBC, CBG, CBN, CBL, THCV and CBDV. According to an embodiment said therapeutic effect treats inflammation, oxidative stress and/or paresthesia and said product comprises CBD and optionally THC at CBD to THC weight/weight ratio greater than 1, greater than 2, greater than 3, greater than 4 or greater than 5. According to an embodiment said therapeutic effect treats inflammation, oxidative stress and/or paresthesia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammation, oxidative stress and/or paresthesia and said the primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof. According to an embodiment, the product comprises at least 2 of these terpenes, at least 3, at least 4, or at least 5 of the terpenes. According to an embodiment, the primary terpene is selected from the group consisting of myrcene, limonene, thymol and combinations thereof. According to an embodiment, the primary terpene is selected from the group consisting of caryophyllene, pinene, linalool, humulene, myrcene, limonene, thymol and combinations thereof. According to various embodiments said primary terpene comprises caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, n and/or nerolidol. According to an embodiment, said therapeutic effect treats inflammation, oxidative stress and/or paresthesia and the product further comprises a compound selected from the group consisting of linalyl acetate, geranyl acetate, eugenol, eugenyl acetate, piperine and combinations thereof. According to an embodiment, the product optionally includes fenchol and/or borneol, and fenchol and/or borneol forms less than 5% by weight of the total terpene content.

According to an embodiment said therapeutic effect treats cardiovascular disease, palpitation and tachycardia and said administered product comprises at least one of CBD, THC, CBC, CBG, CBN, CBL, THCV and CBDV. According to an embodiment said therapeutic effect treats cardiovascular disease, palpitation and tachycardia and said product comprises CBD and optionally THC at CBD to THC weight/weight ratio greater than 1, greater than 2, greater than 3, greater than 4 or greater than 5. According to an embodiment said therapeutic effect treats cardiovascular disease, palpitation and tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cardiovascular disease, palpitation and tachycardia and said the primary terpene is selected from the group consisting of linalool, camphene, limonene, pinene, borneol and combinations thereof. According to an embodiment, the product comprises at least 2 of these terpenes, at least 3, at least 4, or all 5 of the terpenes. According to an embodiment, the primary terpene is selected from the group consisting of camphene, limonene, pinene and combinations thereof. According to various embodiments said primary terpene comprises linalool, camphene, limonene, pinene and/or borneol. According to an embodiment said therapeutic effect treats cardiovascular disease, palpitation and tachycardia and said product comprises a herbal extract selected from the group consisting of extracts of Cimicifuga (Actaea) racemosa, crataegus spp, leonurus spp, Passiflora incarnata and combinations thereof.

According to an embodiment, the enhanced therapeutic effect comprises reducing side effects associated with cannabinoid consumption. According to an embodiment, the primary terpene comprises geraniol, and the side effect comprises hypoglycemia and/or reduction of glucose level. According to an embodiment, the primary terpene is selected from the group consisting of pinene, terpinene, linalool, eucalyptol, myrcene, terpineol, limonene, caryophyllene, borneol and combinations thereof, and the side effect comprises fatigue, weakness, dizziness and/or deficits in balance. According to an embodiment, the primary terpene is selected from the group consisting of camphene, limonene, pinene, borneol and combinations thereof, and the side effect comprises palpitation and/or tachycardia. According to an embodiment, the primary terpene is selected from the group consisting of linalool, citronellol, geraniol, menthol, humulene, bisabolol, borneol, fenchol, linalool, limonene, terpineol, pinene, caryophyllene, myrcene, cymene, terpinene and combinations thereof, and the side effect comprises digestive disorders including vomiting and/or diarrhea. According to an embodiment, the primary terpene is selected from the group consisting myrcene, linalool, caryophyllene, eucalyptol, limonene, nerolidol, fenchol, humulene, guaiol, borneol, bisabolol and combinations thereof, and the side effect comprises headache. According to an embodiment, the primary terpene is selected from the group consisting of pinene, eucalyptol, limonene and combinations thereof, and the side effect comprises coughing. According to an embodiment, the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial and combinations thereof, and the side effect comprises impaired fertility. According to an embodiment, the primary terpene is selected from the group consisting of eucalyptol, caryophyllene, limonene, humulene, phytol and combinations thereof, and the side effect comprises increased appetite. According to an embodiment, the primary terpene is selected from the group consisting of pinene, pulegone, eucalyptol, terpineol, cymene, ocimene, limonene, linalool, caryophyllene, humulene, carene and combinations thereof, and the side effect comprises impaired memory, impaired concentration and/or impaired perception. According to an embodiment, the primary terpene is selected from the group consisting of limonene, linalool, caryophyllene, eucalyptol, pinene, myrcene, terpinene, nerolidol, terpineol, geraniol, menthol, citronellol, neral, geraniol, menthol, borneol, phellandrene, bisabolol, fenchol, humulene and combinations thereof, and the side effect comprises anxiety and/or psychotic-like effects.

According to an embodiment, terpene-enriched cannabinoid product is produced by adding isolated terpenes to a Cannabis extract t.

According to an embodiment, the method for treating a patient comprises (i) administering to the patient for a first period of time a first terpene-enriched Cannabis product comprising a first cannabinoid at a first cannabinoid amount and a first primary terpene at a first primary terpene amount, followed by (ii) administering to the patient for a second period of time a second terpene-enriched Cannabis product comprising the first cannabinoid amount and a second primary terpenes at a second primary terpene amount. According to an embodiment, the method further comprises administering to the patient for a third period of time the first terpene-enriched Cannabis product comprising the first cannabinoid amount and a third primary terpene at a third primary terpene amount. According to an embodiment, the third primary terpene is identical to the first primary terpene.

According to an embodiment, the method for treating a patient comprises administering to said subject for a first period of time a first said product comprising a first cannabinoid at a first cannabinoid amount and a first primary terpene at a first primary terpene amount, followed by (i) administering to said subject for a second period of time said product comprising said first cannabinoid at said first cannabinoid amount or less and a second primary terpene at a second primary terpene amount; (ii) administering to said subject for a second period of time said product comprising said first cannabinoid at said first cannabinoid amount or less and a first primary terpene at an increased first primary terpene amount; (iii) administering to said subject for a second period of time said product comprising said first cannabinoid at a said first cannabinoid amount or less, first primary terpene at a first primary terpene amount and at least one secondary terpene; and/or (iv) administering to said subject for a second period of time said product comprising said first cannabinoid at a said first cannabinoid amount or less, first primary terpene at a first primary terpene amount and a second primary terpene.

According to another embodiment, the method for treating a patient comprises (i) administering to the patient a first terpene-enriched Cannabis product comprising a first cannabinoid at a first cannabinoid amount and a first primary terpene at a first primary terpene amount and administering to the patient, at least 2 hours later a second terpene-enriched Cannabis product comprising the first cannabinoid amount and a second primary terpenes at a second primary terpene amount. According to another embodiment, the first terpene-enriched Cannabis product is administered for day time and the second terpene-enriched Cannabis product is administered for night time.

According to another embodiment, the method for treating a patient comprises administering to the patient (i) a first terpene-enriched Cannabis product comprising a first cannabinoid at a first cannabinoid amount and a first primary terpene at a first primary terpene amount and (ii) a second terpene-enriched Cannabis product comprising the first cannabinoid and a second primary terpenes at a second primary terpene amount.

According to an embodiment, the method for treating a patient comprises (i) administering to the patient multiple Cannabis products to find the best working one; (ii) mimicking the best working product by extracting Cannabis plant material to form an extract and blending the extract with suitable terpenes, whereby a mimicking product is formed and (iii) administering to the patient the mimicking product. According to an embodiment, the method further comprises removing terpenes from the extract prior to the blending.

According to an embodiment, the method for treating a patient comprises administering to said patient a product selected from the group consisting of tablets, gel capsules, medical patches, topicals, creams, varnishes, sublingual oils, sprays, edibles, beverages, suppositories including tampons and/or rectal candles, cigarettes, vaporizer liquids, micro and nano-emulsions, preparations containing micro and nano-particle and combinations thereof, containing the administered product. According to an embodiment, the product comprising tablets containing the product. According to an embodiment, the product comprising gel capsules containing the product. According to an embodiment, the product comprising medical patches containing the product. According to an embodiment, the product comprising topicals containing the product. According to an embodiment, the product comprising creams containing the product. According to an embodiment, the product comprising varnishes containing the product. According to an embodiment, the product comprising sublingual oils containing the product. According to an embodiment, the product comprising edibles containing the product. According to an embodiment, the product comprising beverages containing the product. According to an embodiment, the product comprising tampons containing the product. According to an embodiment, the product comprising rectal candles containing the product. According to an embodiment, the product comprising cigarettes containing the product. According to an embodiment, the product comprising vaporizer liquids containing the product.

### Examples 1-58

The Table below presents examples of compositions as described herin.

| | | | content | | | |
|---|---|---|---|---|---|---|
| | | | (weight %) | | | |
| | | primary | | primary | | |
| | cannabinoid | terpene | cannabinoid | terpene | additive | therapeutic effect for |
| 1 | CBD produced | caryophyllene | 3 | 1 | | osteoporosis |
| | from hemp | | | | | |
| 2 | CBD produced | linalool | 10 | 2 | | anxiety |
| | from hemp | | | | | |
| 3 | synthetic THCV | citronellol | 5 | 1 | | depression |
| 4 | synthetic CBG | myrcene | 15 | 5 | extract of | arthritis |
| | | | | | salix alba | |
| 5 | synthetic CBC | camphene | 15 | 3 | | allergy |
| 6 | synthetic CBD | pinene | 25 | 10 | | acne |
| 7 | CBD produced | limonene | 25 | 5 | | candida |
| | from hemp | | | | | |
| 8 | synthetic CBN | linalool | 10 | 2 | extract of | sleep disorder |
| | | | | | melissa | |
| | | | | | officinalis | |
| 9 | synthetic THC | nerolidol | 23 | 5 | | muscle tension |
| 10 | CBD produced | eucalyptol | 20 | 4 | extract of | anxiety |
| | from hemp | | | | bacopa | |
| | | | | | monnieri | |
| 11 | CBD | pinene | 15 | 5 | | fibromyalgia |
| 12 | synthetic THC | eucalyptol | 20 | 10 | extract of | sleep disorder |
| | | | | | melissa | |
| | | | | | officinalis | |
| 13 | THC and/or | pinene | 25 | 10 | | Parkinson's disease |
| | CBD | | | | | |
| 14 | CBD produced | caryophyllene | 5 | 1 | | nociceptive pain |
| | from hemp | | | | | |
| 15 | CBD | caryophyllene | 40 | 20 | | Huntington's disease |
| 16 | synthetic CBD | nerolidol | 30 | 10 | | dystonia |
| 17 | CBD | terpineol | 40 | 10 | | autism |
| 18 | synthetic CBG | linalool | 5 | 1 | | anxiety |
| 19 | CBD produced | terpineol | 20 | 5 | | epilepsy |
| | from hemp | | | | | |
| 20 | THC and/or | nerolidol | 25 | 10 | | myasthenia gravis |
| | CBD | | | | | |
| 21 | CBD produced | terpineol and | 40 | 10 | | depression |
| | from hemp | citronellol and | | | | |
| | | linalool | | | | |
| 22 | THC | citronellol | 40 | 10 | linalyl | Sleep |
| | | | | | acetate | disorder |
| 23 | THC and/or | alpha-pinene | 20 | 10 | extract of | muscle tensions |
| | CBD | | | | boswellia | |
| | | | | | spp | |
| 24 | synthetic THCV | amyrin | 5 | 1 | | neuropathic pain |
| 25 | CBD | limonene | 30 | 5 | | weight gain |
| 26 | THC and/or | eucalyptol | 20 | 5 | | dementia |
| | CBD | | | | | |
| 27 | CBD produced | alpha-pinene | 40 | 5 | | fatigue |
| | from hemp | | | | | |
| 28 | synthetic CBDV | pinene | 3 | 3 | | pain |
| 29 | CBD produced | limonene | 20 | 5 | | addiction |
| | from hemp | | | | | |
| 30 | THC | terpineol and | 25 | 10 | extract of | Sleep |
| | | citronellol and | | | melissa | disorder |
| | | linalool | | | officinalis | |
| 31 | CBD | caryophyllene | 10 | 5 | | addiction |
| 32 | CBD produced | caryophyllene | 15 | 10 | extract of | osteoporosis |
| | from hemp | | | | angelica | |
| | | | | | sinensis | |
| 33 | CBD produced | phellandrene | 10 | 2 | | ulcerative colitis |
| | from hemp | | | | | |
| 34 | synthetic THC | citronellol | 5 | 2 | linalyl | sleep disorder |
| | | | | | acetata | |
| 35 | CBD | eucalyptol | 15 | ′1-20 | | anxiety |
| 36 | CBD produced | sabinene | 10 | 1 | extract of | osteoarthritis |
| | from hemp | | | | angelica | |
| | | | | | sinensis | |
| 37 | synthetic THCV | terpineol | 5 | 2 | | sleep disorder |
| 38 | synthetic CBDV | nerolidol | 5 | 5 | | inflammation |
| 39 | THC and/or | mycrene | 10 | 5 | | muscle tension |
| | CBD | | | | | |
| 40 | THC | eucalyptol | 10 | 2 | extract of | sleep disorder |
| | | | | | melissa | |
| | | | | | officinalis | |
| 41 | synthetic CBDV | phytol | 5 | 2 | | nausea |
| 42 | synthetic THCV | limonene | 5 | 5 | | anxiety |
| 43 | synthetic CBG | eucalyptol | 5 | 1 | | acne |
| 44 | synthetic CBG | pinene | 5 | 1 | extract of | muscle tension |
| | | | | | boswellia | |
| | | | | | spp | |
| 45 | CBD | eucalyptol | 20 | 10 | | weight gain |
| 46 | synthetic THCV | mycrene | 5 | 5 | | headache |
| 47 | CBD | alpha-pinene | 15 | 5 | | fatigue |
| 48 | THC and/or | nerolidol | 10 | 2 | | osteoarthritis |
| | CBD | | | | | |
| 49 | THC and/or | limonene | 20 | 7 | | digestive problems |
| | CBD | | | | | |
| 50 | CBD produced | terpinolene | 10 | 5 | | fatigue |
| | from hemp | | | | | |
| 51 | synthetic CBG | caryophyllene | 5 | 5 | | Crohn's disease |
| 52 | synthetic CBG | terpinene | 3 | 1 | | ulcerative colitis |
| 53 | synthetic CBC | phytol | 5 | 1 | | psoriasis |
| 54 | synthetic CBC | linalool | 8 | 3 | | itchy skin |
| 55 | synthetic CBDV | caryophyllene | 5 | 5 | | headache |
| 56 | CBD produced | limonene | 20 | 5 | | inflammation |
| | from hemp | | | | | |
| 57 | synthetic CBD | p-cymene, | 20 | 5 | piperine | digestive problems |
| | | ocimene | | | | |
| 58 | synthetic CBC | isopulegol | 5 | 2 | | Crohn's disease |

[1] Oil herein refers to a composition containing an extract of a cannabinoids produced via processing Cannabis, hemp, chemical synthesis, biological synthesis, isolation from plant source and combination thereof.

### Example 59: Treating an Elderly Man with Alzheimer Disease

A man at the age of 75 diagnosed with Alzheimer disease is treated by administering CBD oil produced via hemp. The CBD oil was administrated sublingually. The oil contains 12% CBD and a primary terpene containing limonene. Administrating involves 4 droplets per dose, twice per day. Each administrated dose contains 19 mg of CBD and 4 mg limonene.

A child at the age of 12 diagnosed with ADHD is treated by administering CBD oil produced via hemp. The CBD oil was administrated sublingually. The oil contains 18% CBD and a primary terpene containing eucalyptol. Administrating involves 1 droplet per dose, twice a day. Each administrated dose contains 8 mg of CBD and 5 mg eucalyptol.

### Example 60: Treating a Menopause Woman with Insomnia

A woman at the age of 55 with insomnia is treated by administering synthetic CBN tablets. The tablets contain 6% CBN and a primary terpene containing linalool. Administrating involves 2 tablets before bedtime. Each administrated dose contains 9 mg of CBD and 4 mg pinene.

### Example 61: Treating a Woman with Gastrointestinal Pain

A woman at the age of 33 with symptoms of gastrointestinal pain is treated by administering synthetic CBC oil sublingually. The oil contains 5% CBC and a primary terpene containing myrcene. Administrating involves 1 droplet per dose, twice a day. Each administrated dose contains 2 mg of CBC and 2 mg myrcene.

### Example 62: Treating a Child with Epilepsy

A child at the age of 5 diagnosed with severe epilepsy is treated by administering CBD oil produced via hemp. The CBD oil was administrated sublingually. The oil contains 18% CBD and a primary terpene containing linalool. Administrating involves 3 droplets per dose, twice a day. Each administrated dose contains 21.5 mg of CBD and 8 mg linalool.

### Example 63: Treating a Woman with Symptoms of Nausea and Vomiting

A woman at the age of 25 with symptoms of nausea and vomiting is treated by administering synthetic CBDV oil sublingually. The oil contains 12% CBDV and a primary terpene containing citronellol. Administrating involves 3 droplets per dose, twice a day. Each administrated dose contains 14.5 mg of CBDV and 5 mg citronellol.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the claimed embodiments.

What is claimed is:

1. A method of treatment comprising administering to a subject in need thereof a therapeutically effective amount of a product comprising:
(i) at least one isolated or synthetic cannabinoid, wherein at least a fraction of said isolated or synthetic cannabinoid is obtained by a process selected from the group consisting of processing hemp, chemical synthesis, biological synthesis, isolation from a plant source, crystallization and combinations thereof,
(ii) optionally, at least one other cannabinoid;
(iii) at least one primary terpene, and
(iv) optionally at least three secondary terpenes.

2. The method of claim 1, wherein a ratio of a total weight of said primary terpene to a total weight of said cannabinoid is greater than 0.05.

3. The method of claim 1, wherein said treatment is of a condition selected from the group consisting of treating conditions and/or symptoms associated with at least one selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problems, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof.

4. The method of claim 1, wherein said at least one primary terpene is selected from the group consisting of caryophyllene, linalool, eucalyptol, amyrin, borneol, pinene, terpineol, limonene, nerolidol, geraniol, myrcene, humulene, bisabolol, guaiol and combinations thereof, and wherein said treatment is of neuropathic pain.

5. The method of claim 1, wherein said at least one primary terpene is selected from the group consisting of myrcene, linalool, humulene, eucalyptol, caryophyllene, caryophyllene oxide, amyrin, cycloartenol, borneol, sabinene, camphene, nerolidol, terpineol, geraniol, citronellol, camphor, bisabolol, limonene and combinations thereof, and wherein said treatment is of pain.

6. The method of claim 1, wherein said primary terpene is selected from the group consisting of linalool, terpineol, ocimene, myrcene, limonene, caryophyllene and combinations thereof, and wherein said treatment is of epilepsy.

7. The method of claim 1, wherein said primary terpene is selected from the group consisting of myrcene, limonene, caryophyllene, caryophyllene oxide, terpinene, citronellol, linalool, geraniol, Menthol, humulene, amyrin, cycloartenol, pinene, camphene, eucalyptol, terpineol, borneol, bisabolol, nerolidol, fenchol, guaiol and combinations thereof, and wherein said treatment is of a cancer and/or a cancer related symptom.

8. The method of claim 1, wherein said primary terpene is selected from the group consisting of limonene, caryophyllene, pinene, myrcene, humulene, citronellol, eucalyptol, linalool, terpinen-4-ol, ocimene, geraniol, menthol, borneol, bisabolol, fenchol, guaiol and combinations thereof, and wherein said treatment is of autism and/or an autism spectrum disorder.

9. The method of claim 1, wherein said primary terpene is selected from the group consisting of linalool, eucalyptol, terpineol, myrcene, pinene, pulegone, caryophyllene, geraniol, citronellol, menthol, caryophyllene, pinene, borneol, bisabolol, fenchol and combinations thereof, and wherein said treatment is of at least one selected from the group consisting of attention deficit hyperactivity disorder (ADHD), attention deficit disorder (ADD) and obsessive-compulsive disorder (OCD).

10. The method of claim 1, wherein said primary terpene is selected from the group consisting of myrcene, linalool, limonene, pinene, nerolidol, caryophyllene, eucalyptol, geraniol, menthol, borneol, bisabolol, fenchol and combinations thereof, and wherein said treatment is of Tourette syndrome and/or vocal tics.

11. The method of claim 1, wherein said primary terpene is selected from the group consisting of pinene, limonene, caryophyllene, caryophyllene oxide, nerolidol, eucalyptol, terpinene, terpineol, myrcene, cycloartenol, citronellol, amyrin, camphene, cymene, Ocimene, humulene, farnesene, guaiol, eudesmol, friedelin, linalool, camphor, menthol, thymol, carvacrol, eugenol, farnesol, citronellol, geraniol, citral, ionone and combinations thereof and wherein said treatment is of a Candida infection.

12. The method of claim 1, wherein said primary terpene is selected from the group consisting of limonene, linalool, nerolidol, pinene, terpineol, and combinations thereof, and wherein said treatment is of acne.

13. The method of claim 1, wherein said primary terpene is selected from the group consisting of linalool, myrcene, caryophyllene, eucalyptol, terpineol, citronellol, pinene, terpinene, nerolidol, eucalyptol, sabinene, pinene, limonene, borneol, fenchol, bisabolol, humulene, phytol, citral and combinations thereof, and wherein treatment is of insomnia and/or a sleep disorder.

14. The method of claim 1, wherein said primary terpene is selected from the group consisting of limonene, linalool, terpineol, citronellol, eucalyptol, caryophyllene, geraniol, menthol, iso-menthone, germacrene, pinene, cardinol, neral, geranial, myrcene, terpinene, menth-2-en-1-ol, humulene, bisabolol, borneol, fenchol, nerolidol, phellandrene, guaiol and combinations thereof, and wherein said treatment is of at least one condition selected from the group consisting of anxiety, irritability, nervousness, restless, stress, depression, general discomfort and affect disorder or combinations thereof.

15. The method of claim 1, wherein said primary terpene is selected from the group consisting of myrcene, pinene, nerolidol, linalool, caryophyllene, limonene humulene, bisabolol, sabinene, citral, eucalyptol, terpineol, geraniol, fenchol and combinations thereof, and wherein said treatment is of a condition selected from the group consisting of muscle pain, myalgia, spasticity, muscle tension, cramps and spasms or combinations thereof.

16. The method of claim 1, wherein said primary terpene is selected from the group consisting of limonene, cymene, ocimene, terpinene, caryophyllene, citronellol, sabinene, linalool, pinene, myrcene, germacrene, geraniol, nerolidol, menthol, humulene, fenchol, borneol, guaiol, terpineol and combinations thereof, and wherein said treatment is of a condition selected from the group consisting of a digestive problem, an intestinal disorder, gastrointestinal pain, abdominal pain (colic), pelvic pain, constipation and diarrhea or combinations thereof.

17. The method of claim 1, wherein said primary terpene is selected from the group consisting of linalool, citronellol, geraniol, menthol, humulene, bisabolol, borneol, fenchol, linalool, limonene, terpineol, pinene, caryophyllene, myrcene, cymene, terpinene, valencene and combinations thereof, and wherein said treatment is of a condition selected from the group consisting of lack of appetite, nausea, vomiting and anorexia or combinations thereof.

18. The method of claim 1, wherein said primary terpene is selected from the group consisting of myrcene, linalool, caryophyllene, eucalyptol, pinene, nerolidol, sabinene, limonene, terpinene, menth-2-en-1-ol, terpineol, geraniol, guaiol, humulene, bisabolol, borneol, fenchol and combinations thereof, and wherein said treatment is of a condition selected from the group consisting of joint pain, joint stiffness, arthralgia, arthritis and osteoarthritis or combinations thereof.

19. The method of claim 1, wherein said primary terpene is selected from the group consisting of caryophyllene, myrcene, humulene, pinene, linalool, limonene, nerolidol, terpinene, terpineol, geraniol, carene, citronellol, eucalyptol, sabinene, myrtenol, bisabolol, borneol, fenchol and combinations thereof, and wherein said treatment is of a condition selected from the group consisting of hot flashes, night sweat, palpitation, irritability and restlessness or combinations thereof.

20. A method for the preparation of a product comprising at least one cannabinoid and at least one primary terpene, the method comprising:
(i) obtaining at least one isolated or synthetic cannabinoid by a process selected from the group consisting of processing hemp, chemical synthesis, biological synthesis, isolation from a plant source, crystallization and combinations thereof;
(ii) providing at least one primary terpene; and
(iii) blending said at least one isolated or synthetic cannabinoid with said at least one primary terpene.

21. A product comprising at least one cannabinoid and at least one primary terpene, wherein the product is prepared according to the method of claim 20.

22. A therapeutic product comprising
(i) at least one isolated or synthetic cannabinoid, wherein at least a fraction of said isolated or synthetic cannabinoid is obtained by a process selected from the group consisting of processing hemp, chemical synthesis, biological synthesis, isolation from a plant source, crystallization and combinations thereof,
(ii) optionally, at least one other cannabinoid;
(iii) at least one primary terpene, and
(iv) optionally at least three secondary terpenes,
wherein a weight/weight ratio of total weight of primary terpenes to total weight of cannabinoids is greater than 0.05.

23. The product of claim 22, further comprising at least one herbal preparation, wherein said herbal preparation is produced by at least one of distilling and extracting.
